(19) United States
(12) Patent Application Publication
(10) Pub. No.: US 2006/0122184 A1
(43) Pub. Date: Jun. 8, 2006

(54) Cyanomethyl derivatives as cysteine protease inhibitors

(75) Inventors: Michael Graupe, Pacifica, CA (US); Agnes Lau, San Francisco, CA (US); John O. Link, San Francisco, CA (US); Yang Liu, Foster City, CA (US); Craig J. Mossman, Campbell, CA (US); John W. Patterson, Mountain View, CA (US); Sheila M. Zipfel, Mountain View, CA (US)

Correspondence Address: CELERA, AN APPLERA CORPORATION BUSINESS, 180 KIMBALL WAY, SOUTH SAN FRANCISCO, CA, 94080, US

(73) Assignee: AXYS Pharmaceuticals, Inc., So. San Francisco, CA (US)

(21) Appl. No.: 10/536,889

(22) PCT Filed: Nov. 26, 2003
(86) PCT No.: PCT/US03/37979

§ 371 Date: Oct. 17, 2005

Related U.S. Application Data
(60) Provisional application No. 60/431,354, filed on Dec. 5, 2002.

(51) Int. Cl. A61K 31/53; A61K 31/50; A61K 31/4965; A61K 31/421; A61K 31/381; A61K 31/277; A61K 31/415

(52) U.S. Cl. 514/242; 514/247; 514/255.05; 514/357; 514/374; 514/406; 514/438; 514/521; 544/224; 544/406; 546/330; 548/236; 548/377.1; 558/410; 544/182

(57) ABSTRACT

The present invention is directed to cyanomethyl derivatives that are inhibitors of cysteine protease, in particular, cathepsin B, K, F, and S and are therefore useful in treating diseases mediated by these proteases. The present invention is directed to pharmaceutical compositions comprising these compounds and processes for preparing them.

### BACKGROUND OF THE INVENTION

1. Field of Invention

The present invention is directed to compounds that are inhibitors of cysteine proteases, in particular, cathepsins B, K, L, F, and S and are therefore useful in treating diseases mediated by these proteases. The present invention is directed to pharmaceutical compositions comprising these compounds and processes for preparing them.

2. State of the Art

Cysteine proteases represent a class of peptidases characterized by the presence of a cysteine residue in the catalytic site of the enzyme. Cysteine proteases are associated with the normal degradation and processing of proteins. The aberrant activity of cysteine proteases, e.g., as a result of increased expression or enhanced activation, however, may have pathological consequences. In this regard, certain cysteine proteases are associated with a number of disease states, including arthritis, muscular dystrophy, inflammation, tumor invasion, glomerulonephritis, malaria, periodontal disease, metachromatic leukodystrophy and others. For example, increased cathepsin B levels and redistribution of the enzyme are found in tumors; thus, suggesting a role for the enzyme in tumor invasion and metastasis. In addition, aberrant cathepsin B activity is implicated in such disease states as rheumatoid arthritis, osteoarthritis, pneunocystis carinii, acute pancreatitis, inflammatory airway disease and bone and joint disorders.

The prominent expression of cathepsin K in osteoclasts and osteoclast-related multinucleated cells and its high collagenolytic activity suggest that the enzyme is involved in ososteoclast-mediated bone resorption and, hence, in bone abnormalities such as occurs in osteoporosis. In addition, cathepsin K expression in the lung and its elastinolytic activity suggest that the enzyme plays a role in pulmonary disorders as well.

Cathepsin L is implicated in normal lysosomal proteolysis as well as several disease states, including, but not limited to, metastasis of melanomas. Cathepsin S is implicated in Alzheimer's disease and certain autoimmune disorders, including, but not limited to juvenile onset diabetes, multiple sclerosis, pemphigus vulgaris, Graves' disease, myasthenia gravis, systemic lupus erythemotasus, rheumatoid arthritis and Hashimoto's thyroiditis. In addition, cathepsin S is implicated in: allergic disorders, including, but not limited to asthma; and allogeneic immune reponses, including, but not limited to, rejection of organ transplants or tissue grafts.

Another cysteine protease, Cathepsin F, has been found in macrophages and is involved in antigen processing. It is believed that Cathepsin F in stimulated lung macrophages and possibly in other antigen presenting cells that could play a role in airway inflammation (see G. P. Shi et al, J. Exp. Med. 191,1177, 2000)

In view of the number of diseases wherein it is recognized that an increase in cysteine protease activity contributes to the pathology and/or symptomatology of the disease, molecules which inhibit the activity of this class of enzymes, in particular molecules which inhibitor cathepsins B, K, L, F, and/or S, will therefore be useful as therapeutic agents.

### SUMMARY OF THE INVENTION

In one aspect, this invention is directed to a compound of Formula (I): 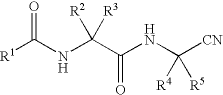
wherein:

R¹ is a group of formula: 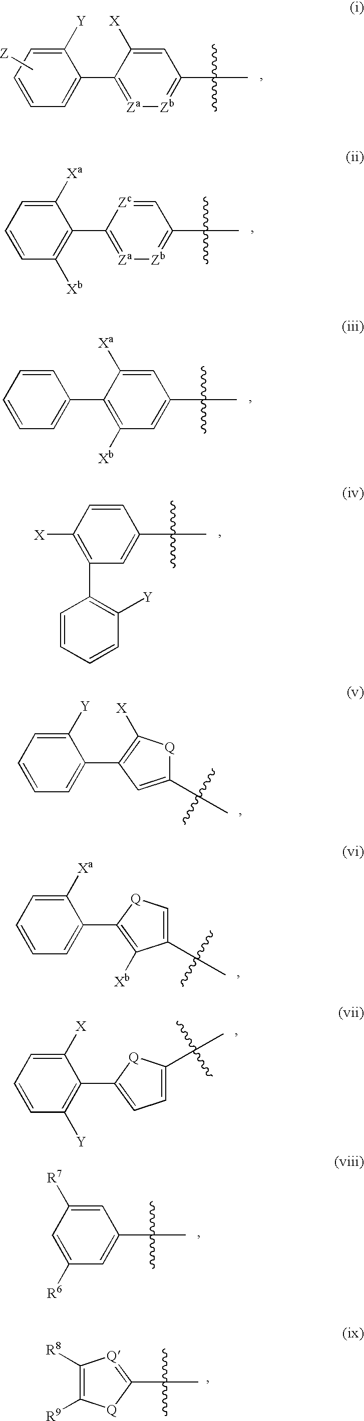 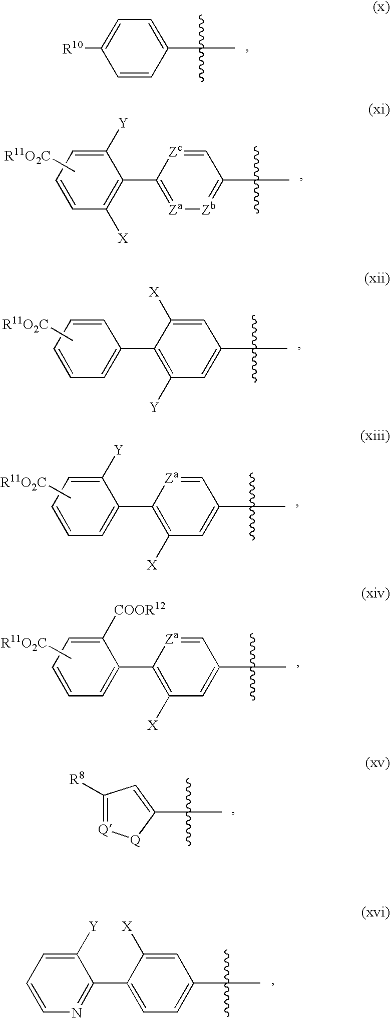
(xvii) 1-(4-aminosulfonylphenyl)-5-(4-chlorophenyl)pyrazol-3-yl;
(xviii) 1-methyl-1H-thieno[2,3-c]pyrazol-5-yl where the 3-position of the pyrazole ring is substituted with alkyl, haloalkyl, or phenyl optionally substituted with alkyl, halo, haloalkyl, haloalkoxy, or alkoxy;
(xix) 4-(3,5-dimethyloxazol-4-yl)phenyl; or
(xx) 4-(5-carboxy-2-methylthiophen-3-yl)phenyl;
(xxi) biphen-4-yl;
(xxii) 4-alkoxycarbonylbiphen-4-yl;
(xxii) 4-carboxybiphen-4-yl; 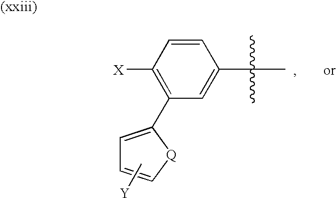
(xxiv) 4-(5-carboxy-2-halothiophen-3-yl)phenyl;
where:

Z^{a} and Z^{b} are independently —CX— or —N— and Z^{c} is selected from —CH— and —N— provided that if an R¹ group contains Z^{a}, Z^{b}, and Z^{c} simultaneously then, when Z^{c} is —N—, then Z^{a} is —N— or —CX— and Z^{b} is —H—; and when Z^{b} is —N— then both Z^{a} and Z^{c} cannot be —N— simultaneously;

Q is —NR— where R is hydrogen or alkyl, —O—, or —S—;

Q′ is —CH— or —N—;

X and Y are independently selected from hydrogen, halo, alkyl, alkoxy, haloalkyl, or haloalkoxy provided that both X and Y are not simultaneously hydrogen;

Z is hydrogen, halo, alkyl, alkoxy, haloalkyl, or haloalkoxy;

X^{a} and X^{b} are independently selected from alkyl, halo, alkoxy, haloalkyl, or haloalkoxy;

R² is selected from the group consisting of hydrogen, cyclopentyl, cyclohexyl, cycloheptyl, methyl, ethyl, n-propyl, 2-propyl, 2-methylpropyl, 2-ethylbutyl, 3-methylbutyl, thiazolylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2,4,4-trimethylpentyl, 1-methylindol-3-ylmethyl, 4-methylindol-3-ylmethyl, 2-napth-1-ylpropyl, benzyloxymethyl, 2-cyclohexylpropyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), benzyl (where the phenyl ring in the benzyl group is optionally substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino), heteroaryl(C₃₋₆)alkyl, and 1-heteroaryl(C₃₋₆)cycloalkylmethyl, and furthermore wherein the alkyl chain in the above groups is optionally substituted with one to six halo;

R³ is hydrogen; or

R² and R³ together with the carbon atom to which they are attached form (C₄₋₈)-cycloalkylene, (C₄₋₈)cycloalkenylene or spirocycloalkylene wherein said (C₄₋₈)cycloalkylene, (C₄₋₈)cycloalkenylene or spirocycloalkylene is optionally substituted with one or two alkyl, alkylidene, or alkenyl;

R⁴ is hydrogen;

R⁵ is hydrogen, alkyl or heteroaryl optionally substituted with alkyl, halo, haloalkyl, haloalkoxy, or alkoxy; or

R⁴ and R⁵ together with the carbon atom to which they are attached form cycloalkylene or heterocycloalkylene;

R⁶ and R⁷ are independently selected from phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl;

R⁸ is phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl;

R⁹ is halo, phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl;

R¹⁰ is a branched alkyl chain of 4-6 carbon atoms or trifluoroalkoxy; and

each R¹¹ and R¹² are independently hydrogen or alkyl;

or a pharmaceutically acceptable salt thereof.

Preferably, the compounds of the invention are represented by Formula (Ia): 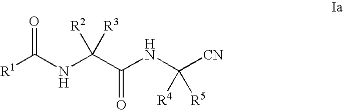
wherein:

R¹ is a group of formula: 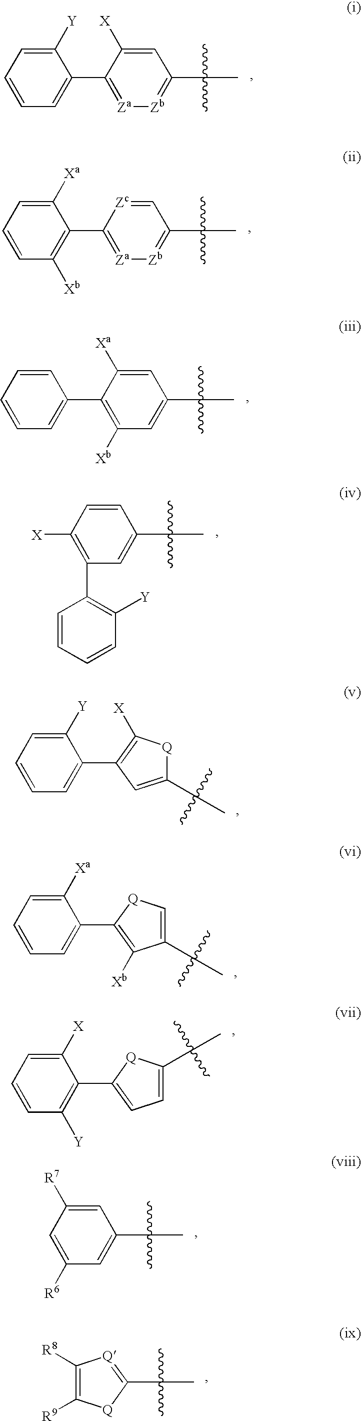 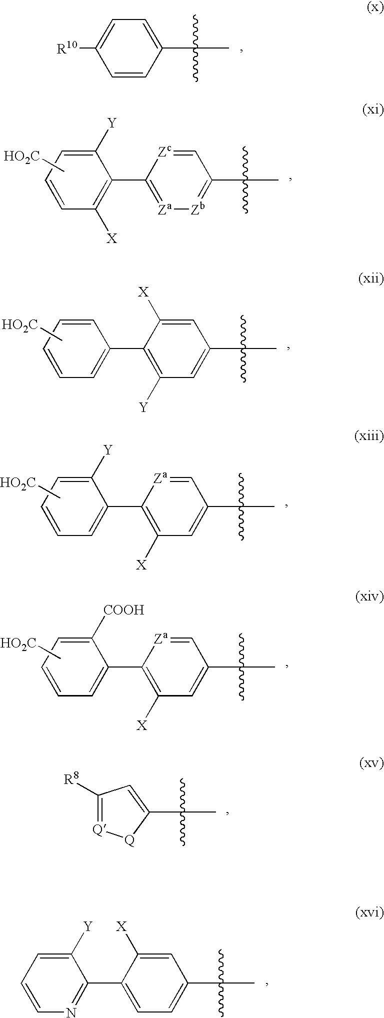
(xvii) 1-(4-aminosulfonylphenyl)-5-(4-chlorophenyl)pyrazol-3-yl;
(xviii) 1-methyl-3-trifluoromethyl-1H-thieno[2,3-c]pyrazol-5-yl;
(xix) 4-(3,5-dimethyloxazol-4-yl)phenyl; or
(xx) 4-(5-carboxy-2-methylthiophen-3-yl)phenyl;
where:

Z^{a} is —CX— or —N— and Z^{b} and Z^{a} are independently selected from —CH— and —N— provided that if an R¹ group contains Z^{a}, Z^{b}, and Z^{c} simultaneously then, when Z^{c} is —N—, then Z^{a} is —N— or —CX— and Z^{b} is —H—; and when Z^{b} is —N— then both Z^{a} and Z^{c} cannot be —N— simultaneously;

Q is —NR— where R is hydrogen or alkyl, —O—, or —S—;

Q′ is —CH— or —N—;

X and Y are independently selected from hydrogen, halo, alkyl, alkoxy, haloalkyl, or haloalkoxy provided that both X and Y are not simultaneously hydrogen;

X^{a} and X^{b} are independently selected from alkyl, halo, alkoxy, haloalkyl, or haloalkoxy;

R² is selected from the group consisting of hydrogen, cyclopentyl, cyclohexyl, cycloheptyl, methyl, ethyl, n-propyl, 2-propyl, 2-methylpropyl, 2-ethylbutyl, 3-methylbutyl, thiazolylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), benzyl (where the phenyl ring in the benzyl group is optionally substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino), heteroaryl(C₃₋₆)alkyl, and 1-heteroaryl(C₃₋₆)cycloalkylmethyl, and furthermore wherein the alkyl chain in the above groups is optionally substituted with one to six halo;

R³ is hydrogen; or

R² and R³ together with the carbon atom to which they are attached form (C₄₋₈)-cycloalkylene, (C₄₋₈)cycloalkenylene or spirocycloalkylene wherein said (C₄₋₈)cycloalkylene, (C₄₋₈)cycloalkenylene or spirocycloalkylene is optionally substituted with one or two alkyl, alkylidene, or alkenyl;

R⁴ is hydrogen;

R⁵ is hydrogen or alkyl; or

R⁴ and R⁵ together with the carbon atom to which they are attached form cycloalkylene or heterocycloalkylene;

R⁶ and R⁷ are independently selected from phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl;

R⁸ and R⁹ are independently selected from phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl; and

R¹⁰ is a branched alkyl chain of 4-6 carbon atoms or trifluoroalkoxy;

or a pharmaceutically acceptable salt thereof.

The compounds of Formula (Ia) are a subset of compounds of Formula (I).

In a second aspect, this invention is directed to a pharmaceutical composition comprising a compound of Formula (I) or a pharmaceutical acceptable salt thereof and a pharmaceutically acceptable excipient.

In a third aspect, this invention is directed to a method of treating a disease in a patient mediated by cathepsins B, K, L, F, and/or S, preferably cathepsin F, which method comprises administering to said patient a pharmaceutical composition comprising a compound of Formula (I) or a pharmaceutical acceptable salt thereof and a pharmaceutically acceptable excipient. Preferably the disease is Alzheimer's disease, respiratory disease such as asthma, osteoporosis, atherosclerosis, restenosis, and autoimmune diseases such as rheumatoid arthritis, systemic lupus erythematosus, Crohn's disease, ulcerative colitis, multiple sclerosis, Guillain-Barre Syndrome, psoriasis, Grave's disease, myasthenia gravis, scleroderma, glomrulonenephritis, dermatitis, endometriosis or insulin dependent diabetes mellitus.

Use of a compound of Formula (I) or (Ia) in the preparation of a medicament for the treatment of a disease mediated by cathepsin F.

### DETAILED DESCRIPTION OF THE INVENTION

Definitions:

Unless otherwise stated, the following terms used in the specification and claims are defined for the purposes of this Application and have the following meanings:

“Alkyl” means a linear saturated monovalent hydrocarbon radical of one to six carbon atoms or a branched saturated monovalent hydrocarbon radical of three to six carbon atoms, unless otherwise indicated, e.g., methyl, ethyl, propyl, 2-propyl, butyl (including all isomeric forms), pentyl (including all isomeric forms), and the like.

“Alkylene” means a linear saturated divalent hydrocarbon radical of one to six carbon atoms unless otherwise stated, e.g., (C₂₋₄)alkylene includes, but is not limited to, groups such as ethylene, propylene, 2-propylene, and butylene.

“Alkenyl” means a linear monovalent hydrocarbon radical of two to six carbon atoms or a branched monovalent hydrocarbon radical of three to six carbon atoms containing one or two double bonds, e.g., ethenyl, propenyl, 2-propenyl, butenyl (including all isomeric forms), and the like.

“Alkylidene” means a straight or branched, unsaturated aliphatic divalent radical having the number of carbon atoms indicated e.g., (C₁₋₆)alkylidene includes (═CH₂), (═CHCH₃), (═CHCH₂CH₃), and the like. Preferably, (═CH₂).

“Alkoxy” means a radical —OR where R is alkyl as defined above, e.g., methoxy, ethoxy, propoxy, or 2-propoxy, n-, iso-, or tert-butoxy, and the like, preferably methoxy.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxyalkyloxy” means a radical —O-(alkylene)OR where R is alkyl as defined above, e.g., methoxymethyloxy, ethoxymethyloxy, 2-methoxyethyloxy, or 2-propoxyethyloxy, and the like.

“Alkoxyalkylthio” means a radical —S—(alkylene)OR where R is alkyl as defined above, e.g., methoxymethylthio, ethoxymethylthio, 2-methoxyethylthio, or 2-propoxyethylthio, and the like.

“Aminoalkyloxy” means a radical —O-(alkylene)NRR′ where R and R′ are independently hydrogen or alkyl as defined above, e.g., methylaminoethyloxy, dimethylaminoethyloxy, and the like.

“Aminoalkylthio” means a radical —S-(alkylene)NRR′ where R and R′ are independently hydrogen or alkyl as defined above, e.g., methylaminoethylthio, dimethylaminoethylthio, and the like.

“Alkylthio” means a radical —SR where R is alkyl as defined above, e.g., methylthio, ethylthio, and the like.

“Alkylsulfinyl” means a radical —S(O)R where R is alkyl as defined above, e.g., methylsulfinyl, ethylsulfinyl, and the like.

“Alkylsulfonyl” means a radical —S(O)₂R where R is alkyl as defined above, e.g., methylsulfonyl, ethylsulfonyl, and the like.

“Alkylamino” means a radical —NHR where R is alkyl as defined above, e.g., methylamino, ethylamino, and the like.

“Aryl” means a monovalent monocyclic or bicyclic aromatic hydrocarbon radical of 6 to 12 ring atoms, and optionally substituted independently with one or more substituents, preferably one, two, or three substituents, selected from alkyl, haloalkyl, alkoxy, alkylthio, halo, nitro, —COR (where R is alkyl), cyano, amino, alkylamino, dialkylamino, hydroxy, carboxy, or —COOR where R is alkyl. Representative examples include, but are not limited to, phenyl, biphenyl, 1-naphthyl, and 2-naphthyl and the derivatives thereof

“Aralkyl” means a radical -(alkylene)-R where R is an aryl group as defined above e.g., benzyl, phenylethyl, 3-(3-chlorophenyl)-2-methylpentyl, and the like.

“Cycloalkyl” means a cyclic monovalent saturated monovalent hydrocarbon radical of three to six carbon atoms unless otherwise indicated e.g., cyclopropyl, cyclobutyl, and the like, preferably cyclopropyl.

“Cycloalkylene” means a cyclic saturated divalent hydrocarbon radical of three to eight carbon atoms unless otherwise indicated e.g., cyclopropylene, cyclobutylene, cycloheptylene, and the like.

“Cycloalkylalkyl” means a radical -(alkylene)cycloalkyl e.g., cyclopropylmethyl, cyclobutylmethyl, and the like, preferably cyclopropylmethyl.

“Cycloalkenylene” means a cyclic unsaturated divalent hydrocarbon radical of three to six carbon atoms unless otherwise indicated e.g., cyclopropenylene, cyclobutenylene, and the like.

“Dialkylamino” means a radical —NRR′ where R and R′ are independently alkyl as defined above, e.g., dimethylamino, methylethylamino, and the like.

“Halo” means fluoro, chloro, bromo, and iodo, preferably fluoro or chloro.

“Haloalkyl” means alkyl substituted with one or more halogen atoms, preferably one to three halogen atoms, preferably fluorine or chlorine, including those substituted with different halogens, e.g., —CH₂Cl, —CF₃, —CHF₂, and the like, preferably trifluoromethyl.

“Haloalkoxy” means a radical —OR where R is haloalkyl as defined above, e.g., trifluoromethoxy, 2,2,2-trifluoroethoxy, difluoromethoxy, and the like, preferably trifluoromethoxy.

“Heterocycloalkyl” means a saturated or unsaturated monovalent cyclic group of 3 to 8 ring atoms in which one or two ring atoms are heteroatoms selected from N, O, or S(O)n, where n is an integer from 0 to 2, the remaining ring atoms being C. The heterocycloalkyl ring may be optionally substituted with one or more substituents, preferably one or two substituents, independently selected from alkyl, cycloalkyl, cycloalkylalkyl, aryl, heteroaryl, aralkyl, heteroaralkyl, halo, haloalkyl, hydroxy, hydroxyalkyl, alkoxy, alkoxyalkyl, halo, cyano, carboxy, or —COOR where R is alkyl as define above or a protected derivative thereof. More specifically the term heterocycloalkyl includes, but is not limited to, pyrrolidino, piperidino, morpholino, piperazino, tetrahydropyranyl, and thiomorpholino.

“Heterocycloalkylene” means a saturated or unsaturated divalent cyclic group of 3 to 8 ring atoms in which one or two ring atoms are heteroatoms selected from N, O, or S(O)n, where n is an integer from 0 to 2, the remaining ring atoms being C. The heterocycloalkylene ring may be optionally substituted with one or more substituents, preferably one or two substituents, independently selected from alkyl, cycloalkyl, cycloalkylalkyl, aryl, heteroaryl, aralkyl, heteroaralkyl, halo, haloalkyl, hydroxy, hydroxyalkyl, alkoxy, alkoxyalkyl, halo, cyano, carboxy, or —COOR where R is alkyl as define above or a protected derivative thereof. More specifically the term heterocycloalkylene includes, but is not limited to, groups such as: 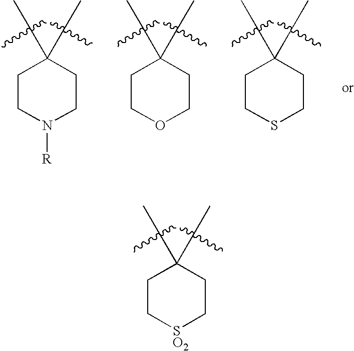
where R is hydrogen, alkyl, cycloalkylalkyl or haloalkyl.

“Heteroaryl” means a monovalent monocyclic or bicyclic aromatic radical of 5 to 10 ring atoms containing one or more, preferably one or two ring heteroatoms selected from N, O, or S, the remaining ring atoms being carbon. The heteroaryl ring is optionally substituted with one or more substituents, preferably one or two substituents, independently selected from alkyl, haloalkyl, alkoxy, alkylthio, halo, nitro, cyano, amino, alkyl or dialkylamino, hydroxy, carboxy, or —COOR where R is alkyl as define above. More specifically the term heteroaryl includes, but is not limited to, pyridyl, pyrrolyl, imidazolyl, thienyl, furanyl, indolyl, quinolyl, pyrazine, pyrimidine, pyradizine, oxazole, isooxazolyl, benzoxazole, quinoline, isoquinoline, benzopyranyl, and thiazolyl.

“Heteroaralkyl” means a radical -(alkylene)-R where R is a heteroaryl group as defined above e.g., pyridylmethyl, furanylmethyl, indolylmethyl, pyrimidinylmethyl, and the like.

“1-Heteroaryl(C₃₋₆)cycloalkylmethyl” means a radial of the formula: 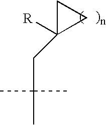
where R is a heteroaryl group as defined above and n is 1, 2, 3 or 4. Representative examples include, but are not limited to, 1-pyridin-2-ylcyclopropylmethyl, 1-pyridin-2-ylcyclobutylmethyl, and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

The present invention also includes the prodrugs of compounds of Formula (I). The term prodrug is intended to represent covalently bonded carriers, which are capable of releasing the active ingredient of Formula (I) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups by routine manipulation or in vivo. Prodrugs of compounds of Formula (I) are also within the scope of this invention.

The present invention also includes N-oxide derivatives and protected derivatives of compounds of Formula (I). For example, when compounds of Formula (I) contain an oxidizable nitrogen atom, the nitrogen atom can be converted to an N-oxide by methods well known in the art or in vivo. For example, the nitrogen atom in a pyridyl group in a compound of Formula (I) can be oxidized to give a corresponding pyridyl-N-oxide compound of Formula (I).

A “pharmaceutically acceptable salt” of a compound means a salt that is pharmaceutically acceptable and that possesses the desired pharmacological activity of the parent compound. Such salts include:

acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All enantiomeric, diastereomeric, and racemic forms are within the scope of this invention, unless the specific stereochemistry or isomeric form is specifically indicated.

Additionally, as used herein the terms alkyl includes all the possible isomeric forms of said alkyl group albeit only a few examples are set forth.

“Optional” or “optionally” means that the subsequently described event or circumstance may but need not occur, and that the description includes instances where the event or circumstance occurs and instances in which it does not. For example, the term “phenyl group optionally with an alkyl group” means that the alkyl may but need not be present, and the description includes situations where the phenyl group is substituted with an alkyl group and situations where the phenyl group is not substituted with the alkyl group.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Spirocycloalkylene” means a saturated divalent polycyclic ring system containing from seven or eight ring carbon atoms that are bonded in such a way that a single carbon atom is common to both rings. Examples include, but are not limited to, rings such as:

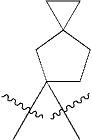

“Treating” or “treatment” of a disease includes:

(1) preventing the disease, i.e. causing the clinical symptoms of the disease not to develop in a mammal that may be exposed to or predisposed to the disease but does not yet experience or display symptoms of the disease;

(2) inhibiting the disease, i.e., arresting or reducing the development of the disease or its clinical symptoms; or

(3) relieving the disease, i.e., causing regression of the disease or its clinical symptoms.

A “therapeutically effective amount” means the amount of a compound of Formula (I) that, when administered to a mammal for treating a disease, is sufficient to effect such treatment for the disease. The “therapeutically effective amount” will vary depending on the compound, the disease and its severity and the age, weight, etc., of the mammal to be treated.

In Tables 1-4 below the positions around the biphenyl and pyridyl moieties in R¹ group of compounds of Formula (I) are numbered as shown below.

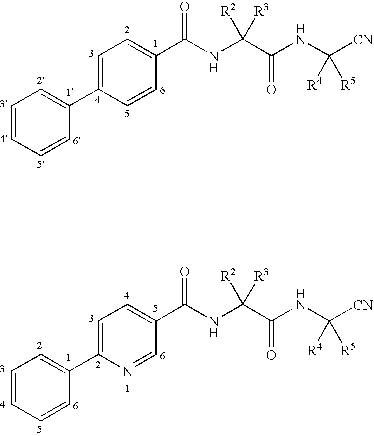

Representative compounds of Formula I are listed in Tables 1-4 below:

TABLE 1

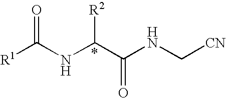

| Cpd. # | Mass Spec. | Stereochem. at (C*) | R¹ | R² |
|---|---|---|---|---|
| 1 | | (S) | 2′-Cl-biphen-4-yl | 2,6-diF-benzyl |
| 2 | | (S) | 2′-Cl-biphen-4-yl | 2,6-diF-4-OCH₃benzyl |
| 3 | | (S) | 2-(2-Clphenyl)pyridin-5-yl | 2,6-diF-benzyl |
| 4 | | (S) | 2′,3-diCl-biphen-4-yl | 2,6-diF-benzyl |
| 5 | | (S) | 2′-Cl-biphen-4-yl | 2(S)-phenylpropyl |
| 6 | | (S) | 3,5-di(2-methoxyphenyl)-phenyl | 2-methylpropyl |
| 7 | | (S) | 2,3-di(2-methylphenyl)-thiophen-5-yl | 2-methylpropyl |
| 8 | | (S) | 3-(2-chlorophenyl)-isoxazol-5-yl | 2-methylpropyl |
| 9 | | (S) | 5′-carboxy-2′-methyl-biphen-4-yl | 2-methylpropyl |
| 10 | | (S) | 5′-carboxy-2′-chloro-biphen-4-yl | 2-methylpropyl |
| 11 | | (S) | 4′-carboxy-2′-chlorobiphen-4-yl | 2-methylpropyl |
| 12 | | (RS) | 2,3-diphenylthiophen-5-yl | 2,6-diF-benzyl |
| 13 | | (S) | 2′-chlorobiphen-4-yl | 2(R)-phenylpropyl |
| 14 | | (RS) | 2′-chlorobiphen-4-yl | 2-methyl-2-phenylpropyl |
| 15 | | (RS) | 2′-chlorobiphen-4-yl | 2-phenylethyl |
| 16 | | (S) | 2′-chlorobiphen-4-yl | phenylprop-2-enyl |
| 17 | | (S) | 2-(2-chlorophenyl)pyridin-5-yl | 2-methylpropyl |
| 18 | | (S) | 2-(2,6-dichlorophenyl)-pyridin-5-yl | 2-methylpropyl |
| 19 | | (S) | 2-(2-CF₃phenyl)pyridin-5-yl | 2-methylpropyl |
| 20 | | (S) | 2-(5-carboxy-2-chlorophenyl)-pyridin-5-yl | 2-methylpropyl |
| 21 | | (S) | 2-(2,6-dichlorophenyl)-3-chloropyridin-5-yl | 2-methylpropyl |
| 22 | | (S) | 1-methyl-3-trifluoromethyl-1H-thieno[2,3-c]pyrazol-4-yl | 2-methylpropyl |
| 23 | | (S) | 1-methyl-3-trifluoromethyl-1H-thi- | 2,6-diF-benzyl |
| | | | eno[2,3-c]pyrazol-4-yl | |
| 24 | | (RS) | 4-(3-methylpyridin-2-yl)-phenyl | 2,6-diFbenzyl |
| 25 | | (S) | 4-(5-carboxy-2-methylthiophen-3-yl)phenyl | 2-methylpropyl |
| 26 | | (S) | 2,3-diphenylthiophen-5-yl | benzyl |
| 27 | | (S) | 2′-chlorobiphen-4-yl | benzyl |
| 28 | | | 2,3-diphenylthiophen-5-yl | H |
| 29 | | (S) | 5′-carboxy-2′-chloro-biphen-4-yl | 2S-phenylpropyl |
| | 490 (M + 1) | | | |
| 30 | | (S) | 2-(2-chlorophenyl)pyridin-5-yl | 2,6-diF-4-OCH₃benzyl |
| 31 | | (S) | 1-oxo-2-(2-Clphenyl)pyridin-5-yl | 2-methylpropyl |
| 32 | | (S) | 2′-Clbiphen-4-yl | thiazol-2-ylmethyl |
| 33 | | (S) | 2-(2-Cl-phenyl)pyridin-5-yl | thiazol-2-ylmethyl |
| 34 | | (S) | 3,5-di-(thien-3-yl)phenyl | 2-methylpropyl |
| 35 | | (S) | 2′,6′-diCl-biphen-4-yl | 2-methylpropyl |
| 36 | | (S) | 3,5-di-(furan-2-yl)phenyl | 2-methylpropyl |
| 37 | | (S) | 4-Cl-3-(2′-methylphenyl)phenyl | 2-methylpropyl |
| 38 | | (S) | 3,5-di-(2′-Clphenyl)phenyl | 2-methylpropyl |
| 39 | | (S) | 4,5-di-(2-Clphenyl)-(thien-2-yl) | 2-methylpropyl |
| 40 | | (RS) | 2′-Cl-biphen-4-yl | 2,6-diFphenylmethyl |
| 41 | | (S) | 4-(2-Cl-phenyl)-(thien-2-yl) | 2-methylpropyl |
| 42 | | (RS) | 2′-Cl-biphen-4-yl | 4-methyl-1H-indol-3-ylmethyl |
| 43 | | (S) | 3-Cl-2′-methylbiphen-4-yl | 2-methylpropyl |
| 44 | | (S) | 2-(2,6-diCl-phenyl)-1-oxo-pyridin-5-yl | 2-methylpropyl |
| 45 | | (S) | 2-(2-methylphenyl)-1-oxo-pyridin-5-yl | 2-methylpropyl |
| 46 | | (S) | 2′-methyl-4′-carboxy-biphen-4-yl | 2-methylpropyl |
| 47 | | (S) | 2′-F-4′-carboxy-biphen-4-yl | 2-methylpropyl |
| 48 | | (S) | 3-(2-Cl-phenyl)isoxazol-5-yl | 2,6-difluorophenylmethyl |
| 49 | | (S) | 3-Cl-2-(2-Cl-phenyl)pyridin-5-yl | 2-methylpropyl |
| 50 | | (S) | 3-Cl-2-(2-Cl-phenyl)-1-oxo-pyridin-5-yl | 2-methylpropyl |
| 51 | | (S) | 4-[5-carboxy-2-Cl-(thien-3-yl)]-phenyl | 2-methylpropyl |
| 52 | | (S) | 3-Cl-2-(2-tri- | 2-methylpropyl |
| | | | fluoromethylphenyl)pyridin-5-yl | |
| 53 | | (S) | 3-Cl-2-(2-methylphenyl)pyridin-5-yl | 2-methylpropyl |
| 54 | 447 (M + 1) | (S) | 2-(2-Clphenyl)-1-oxo-pyridin-5-yl | 2S-phenylpropyl |
| 55 | | (S) | 2-(4-carboxy-2-Cl-phenyl)pyridin-5-yl | 2-methylpropyl |
| 56 | | (S) | 2-(2-Cl-phenyl)-1-oxo-pyridin-5-yl | 2S-phenylpropyl |
| 57 | | (S) | 5′-carboxy-2′-Cl-biphen-4-yl | 2,6-diF-benzyl |
| 58 | | (S) | 4-[5-carboxy-2-methyl(thien-3-yl)]phenyl | 2,6-diF-benzyl |
| 59 | 477 (M + 1) | (S) | 2-(2-Cl-phenyl)pyridin-5-yl | 2S-(2-methoxyphenyl)propyl |
| 60 | | (S) | 2′-Cl-biphen4-yl | 2S-(2-methoxyphenyl)propyl |
| 61 | | (S) | 4′-carboxy-2′-Cl-biphen-4-yl | 2,6-diF-benzyl |
| 62 | | (S) | 2′-Cl-biphen4-yl | 2S-(2-trifluoromethoxyphenyl)propyl |
| 63 | 532 (M + 1) | (S) | 4′-carboxy-2′,6′-diCl-biphen-4-yl | 2,6-diF-benzyl |
| 64 | | (S) | 4′-carboxy-2′,6′-diCl-biphen-4-yl | 2-methylpropyl |
| 65 | | (S) | 2′-Cl-biphen4-yl | 2S-phenylpropyl |
| 66 | | (S) | 4-trifluoromethoxyphenyl | 2S-(2-methoxyphenyl)propyl |
| 67 | | (S) | 2-(2,6-diCl-phenyl)pyridin-5-yl | 2S-(2-methoxyphenyl)propyl |
| 68 | | (S) | 1-methyl-3-trifluoromethyl-1H-thi- | 2S-(2-methoxyphenyl)propyl |
| | | | eno[2,3-c]pyrazol-5-yl | |
| 69 | | (S) | 5′-carboxy-2′,3-diCl-biphen-4-yl | 2-methylpropyl |
| 70 | | (S) | 4′-carboxy-2′,3-diCl-biphen-4-yl | 2-methylpropyl |
| 71 | 528 (M + 1) | (S) | 5′-carboxy-2′-Cl-biphen-4-yl | (2,6-difluoro-4-methoxyphenyl)methyl |
| 72 | 510 (M + 1) | (S) | 2′,3-diCl-biphen-4-yl | 2S-(2-methoxyphenyl)propyl |
| 73 | | NA | 2′,3-diCl-biphen-4-yl | H |
| 74 | | (S) | 2′,3-diCl-biphen-4-yl | methyl |
| 75 | | (S) | 2′,3-diCl-biphen-4-yl | 1-methylethyl |
| 76 | | (S) | 1-methyl-3-trifluoromethyl-1H-thi- | 2S-phenylpropyl |
| | | | eno[2,3-c]pyrazol-5-yl | |
| 77 | | (S) | 5′-(methoxycarbonyl)-2′-Cl-biphen-4-yl | (2,6-difluoro-4-methoxyphenyl)methyl |
| 78 | | (S) | biphen-4-yl | 2S-(2-methoxyphenyl)propyl |
| 79 | | (S) | 4′-carboxybiphen-4-yl | 2S-(2-methoxyphenyl)propyl |
| 80 | | (S) | 4′-(methoxycarbonyl)-2′-Cl-biphen-4-yl | (2,6-difluorophenyl)methyl |
| 81 | | (S) | 2-[4-(methoxycarbonyl)-2-Cl-phe- | 2-methylpropyl |
| | | | nyl]pyridin-5-yl | |
| 82 | 504 (M + 1) | (S) | 5′-(methoxycarbonyl)-2′-Cl-biphen-4-yl | 2S-phenylpropyl |
| 83 | | (S) | 2,2′,6′-trifluorobiphen-4-yl | 2-methylpropyl |
| 84 | | (S) | 2,2′-dichlorobiphen-4-yl | 2-methylpropyl |
| 85 | | (S) | 5-chloro-4-(2-chlorophenyl)(thien-2-yl) | 2-methylpropyl |
| 86 | | (S) | 5-chloro-4-phenyl-(thien-2-yl) | 2-methylpropyl |
| 87 | | (S) | 2-(2,5-dimethylphenyl)pyridin-5-yl | 2-methylpropyl |
| 88 | | (RS) | 2′-chlorobiphen-4-yl | 2-(cyclohexyl)propyl |
| 89 | | (S) | 2′-chlorobiphen-4-yl | 1-methylindol-3-ylmethyl |
| 90 | | (S) | 3-[5-methyl(thien-2-yl)]phenyl | 2-methylpropyl |
| 91 | | (S) | 1-methyl-3-phenyl-1H-thieno[2,3-c]py- | 2S-phenylpropyl |
| | | | razol-5-yl | |
| 92 | | (S) | 1,3-dimethyl-1H-thieno[2,3-c]pyrazol-5-yl | 2S-phenylpropyl |

and are named as:
2′-chlorobiphenyl-4-carboxylic acid[1(S)-(cyanomethylcarbamoyl)-2-(2,6-difluorophenyl)ethyl]amide;
2′-chlorobiphenyl-4-carboxylic acid[1(S)-(cyanomethylcarbamoyl)-2-(2,6-difluoro-4-methoxyphenyl)ethyl]amide;
6-(2-chlorophenyl)-N-[1(S)-(cyanomethylcarbamoyl)-2-(2,6-difluorophenyl)ethyl]nicotinamide;
2′,3-dichlorobiphenyl-4-carboxylic acid[1(S)-(cyanomethylcarbamoyl)-2-(2,6-difluorophenyl)ethyl]amide;
2′-chlorobiphenyl-4-carboxylic acid[1(S)-(cyanomethylcarbamoyl)-3-(2S-phenyl)butyl]amide;
3,5-di-(2-methoxyphenyl)phenyl-1-carboxylic acid[1(S)-(cyanomethylcarbamoyl)-3-(methyl)butyl]amide;
2,3-di(methylphenyl)-thienyl-5-carboxylic acid[1(S)-(cyanomethylcarbamoyl)-3-(methyl)butyl]amide;
3-(2-chlorophenyl)-isoxazolyl-5-carboxylic acid[1(S)-(cyanomethylcarbamoyl)-3-(methyl)butyl]amide;
5′-carboxy-2′-methyl-biphenyl-4-carboxylic acid[1(S)-(cyanomethylcarbamoyl)-3-(methyl)butyl]amide;
5′-carboxy-2′-chloro-biphenylcarboxylic acid[1(S)-(cyanomethylcarbamoyl)-3-(methyl)butyl]amide;
4′-carboxy-2′-chloro-biphenyl-4-carboxylic acid[1(S)-(cyanomethylcarbamoyl)-3-(methyl)butyl]amide;
2,3-diphenylthienyl-5-carboxylic acid[1(RS)-(cyanomethylcarbamoyl)-2-(2,6-difluoro-phenyl)ethyl]amide;
2′-chlorobiphenyl-4-carboxylic acid[1(S)-(cyanomethylcarbamoyl)-(3(R)-phenyl)butyl]amide;
2′-chlorobiphenyl-4-carboxylic acid[1(S)-(cyanomethylcarbamoyl)-(3-methyl-3-phenyl)butyl]amide;
2′-chlorobiphenyl-4-carboxylic acid[1(S)-(cyanomethylcarbamoyl)-(3-phenyl)propyl]amide;
2′-chlorobiphenyl-4-carboxylic acid[1(S)-(cyanomethylcarbamoyl)-[(3R-phenyl)but-3-enyl]]amide;
2-(2-chlorophenyl)pyridinyl-5-carboxylic acid[1(S)-(cyanomethylcarbamoyl)-3-(methyl)butyl]amide;
2-(2,6-dichlorophenyl)pyridinyl-5-carboxylic acid[1(S)-(cyanomethylcarbamoyl)-3-(methyl)butyl]amide;
2-(2-trifluoromethylphenyl)pyridinyl-5-carboxylic acid[1(S)-(cyanomethylcarbamoyl)-3-(methyl)butyl]amide;
2-(5-carboxy-2-chlorophenyl)-pyridinyl-5-carboxylic acid[1(S)-(cyanomethylcarbamoyl)-3-(methyl)butyl]amide;
2-(2,6-dichlorophenyl)-3-chloropyridinyl-5-carboxylic acid[1(S)-(cyanomethylcarbamoyl)-3-(methyl)butyl]amide;
1-methyl-3-trifluoro-1H-thieno[2,3-c]pyrazolyl-4-carboxylic acid[1(S)-(cyanomethylcarbamoyl)-3-(methyl)butyl]amide;
1-methyl-3-trifluoromethyl-1H-thieno[2,3-c]pyrazolyl-4-carboxylic acid[1(S)-(cyanomethylcarbamoyl)-2-(2,6-difluorophenyl)ethyl]amide;
4-(3-methylpyridin-2-yl)-phenyl-1-carboxylic acid[1(S)-(cyanomethylcarbamoyl)-2-(2,6-difluorophenyl)ethyl]amide;
4-(5-carboxy-2-methylthien-3-yl)phenyl-1-carboxylic acid[1(S)-(cyanomethylcarbamoyl)-3-(methyl)butyl]amide;
2,3-diphenylthienyl-5-carboxylic acid[1(S)-(cyanomethylcarbamoyl)-2-phenylethyl]amide;
2′-chlorobiphenyl-4-carboxylic acid[1(S)-(cyanomethylcarbamoyl)-2-phenylethyl]amide;
2,3-diphenylthienyl-5-carboxylic acid[1(S)-(cyanomethylcarbamoyl)-methyl]amide;
5′-carboxy-2′-chloro-biphenyl-4-carboxylic acid[1(S)-(cyanomethylcarbamoyl)-3S-(phenyl)butyl]amide;
2-(2-chlorophenyl)pyridinyl-5-carboxylic acid[1(S)-(cyanomethylcarbamoyl)-(2,6-difluoro-4-methoxyphenyl)ethyl]amide;
6-(2-chlorophenyl)-N-[1(S)-(cyanomethylcarbamoyl)-3-methylbutyl]-1-oxynicotinamide;
2′-chlorobiphenyl-4-carboxylic acid[1(S)-(cyanomethylcarbamoyl)-2-(thiazol-2-yl)ethyl]amide;
2-(2-chloro-phenyl)pyridinyl-5-carboxylic acid[1(S)-(cyanomethylcarbamoyl)-2-(thiazol-2-yl)ethyl]amide;
3,5-bis-(thien-3-yl)phenyl-1-carboxylic acid[1(S)-(cyanomethylcarbamoyl)-3-(methyl)butyl]amide;
2′,6′-dichloro-biphenyl-4-carboxylic acid[1(S)-(cyanomethylcarbamoyl)-3-(methyl)butyl]amide;
3,5-bis-(furan-2-yl)phenyl-1-carboxylic acid[1(S)-(cyanomethylcarbamoyl)-3-(methyl)butyl]amide;
4-chloro-3-(2′-methylphenyl)phenyl-1-carboxylic acid[1(S)-(cyanomethylcarbamoyl)-3-(methyl)butyl]amide;
3,5-bis-(2′-chlorophenyl)phenyl-1-carboxylic acid[1(S)-(cyanomethylcarbamoyl)-3-(methyl)butyl]amide;
4,5-bis-(2-chlorophenyl)thienyl-2-carboxylic acid[1(S)-(cyanomethylcarbamoyl)-3-(methyl)butyl]amide;
2′-chlorobiphenyl-4-carboxylic acid[1(S)-(cyanomethylcarbamoyl)-(2,6-difluorophenyl)ethyl]amide;
4-(2-chlorophenyl)thien-yl-2-carboxylic acid[1(S)-(cyanomethylcarbamoyl)-3-(methyl)butyl]amide;
2′-chlorobiphenyl-4-carboxylic acid[1(S)-(cyanomethylcarbamoyl)-2-(4-methyl-1H-indol-3-yl)ethyl]amide;
3-chloro-2′-methylbiphenyl-4-carboxylic acid[1(S)-(cyanomethylcarbamoyl)-3-(methyl)butyl]amide;
2-(2,6-dichloro-phenyl)-1-oxo-pyridinyl-5-carboxylic acid[1(S)-(cyanomethylcarbamoyl)-3-(methyl)butyl]amide;
2-(2-methylphenyl)-1-oxo-pyridinyl-5-carboxylic acid[1(S)-(cyanomethylcarbamoyl)-3-(methyl)butyl]amide;
2′-methyl-4′-carboxy-biphenyl-4-carboxylic acid[1(S)-(cyanomethylcarbamoyl)-3-(methyl)butyl]amide;
2′-fluoro-4′-carboxy-biphenyl-4-carboxylic acid[1(S)-(cyanomethylcarbamoyl)-3-(methyl)butyl]amide;
3-(2-chlorophenyl)isoxazolyl-5-carboxylic acid[1(S)-(cyanomethylcarbamoyl)-(2,6-fluorophenyl)ethyl]amide;
3-chloro-2-(2-chloro-phenyl)pyridinyl-5-carboxylic acid[1(S)-(cyanomethylcarbamoyl)-3-(methyl)butyl]amide;
3-chloro-2-(2-chloro-phenyl)-1-oxo-pyridinyl-5-carboxylic acid[1(S)-(cyanomethylcarbamoyl)-3-(methyl)butyl]amide;
4-[5-carboxy-2-chloro-(thien-3-yl)]-phenyl-1-carboxylic acid[1(S)-(cyanomethylcarbamoyl)-3-(methyl)butyl]amide;
3-chloro-2-(2-trifluoromethylphenyl)pyridinyl-5-carboxylic acid[1(S)-(cyanomethylcarbamoyl)-3-(methyl)butyl]amide;
3-chloro-2-(2-methylphenyl)pyridinyl-5-carboxylic acid[1(S)-(cyanomethylcarbamoyl)-3-(methyl)butyl]amide;
2-(2-chloro-phenyl)-1-oxo-pyridinyl-5-carboxylic acid[1(S)-(cyanomethylcarbamoyl)-3-(2S-phenyl)butyl]amide;
2-(4-carboxy-2-chlorophenyl)pyridinyl-5-carboxylic acid[1(S)-(cyanomethylcarbamoyl)-3-(methyl)butyl]amide;
2-(2-chloro-phenyl)-1-oxo-pyridinyl-5-carboxylic acid[1(S)-(cyanomethylcarbamoyl)-3-(2S-phenyl)butyl]amide;
5′-carboxy-2′-chloro-biphenyl-4-carboxylic acid[1(S)-(cyanomethylcarbamoyl)-(2,6-difluorophenyl)ethyl]amide;
4-[5-carboxy-2-methyl(thien-3-yl)]phenyl-1-carboxylic acid[1(S)-(cyanomethylcarbamoyl)-(2,6-fluorophenyl)ethyl]amide;
2-(2-chloro-phenyl)pyridinyl-5-carboxylic acid[1(S)-(cyanomethylcarbamoyl)-3-(2S-(2-methoxyphenyl))butyl]amide;
2′-chloro-biphenyl-4-carboxylic acid[1(S)-(cyanomethylcarbamoyl)-3-(2S-(2-methoxyphenyl))butyl]amide;
4′-carboxy-2′-chloro-biphenyl-4-carboxylic acid[1(S)-(cyanomethylcarbamoyl)-(2,6-difluorophenyl)ethyl]amide;
2′-chloro-biphenyl-4-carboxylic acid[1(S)-(cyanomethylcarbamoyl)-3-(2S-(2-trifluoromethoxyphenyl))butyl]amide;
4′-carboxy-2′,6′-dichloro-biphenyl-4-carboxylic acid[1(S)-(cyanomethylcarbamoyl)-(2,6-difluorophenyl)ethyl]amide;
4′-carboxy-2′,6′-dichloro-biphenyl-4-carboxylic acid[1(S)-(cyanomethylcarbamoyl)-3-(methyl)butyl]amide;
2′-chloro-biphenyl-4-carboxylic acid[1(S)-(cyanomethylcarbamoyl)-3-(2S-phenyl)butyl]amide;
4-trifluoromethoxyphenyl-1-carboxylic acid[1(S)-(cyanomethylcarbamoyl)-3-(2S-(2-methoxyphenyl))butyl]amide;
2-(2,6-dichloro-phenyl)pyridinyl-5-carboxylic acid[1(S)-(cyanomethylcarbamoyl)-3-(2S-(2-methoxyphenyl))butyl]amide;
1-methyl-3-trifluoromethyl-1H-thieno[2,3-c]pyrazolyl-5-carboxylic acid[1(S)-(cyanomethylcarbamoyl)-3-(2S-(2-methoxyphenyl))butyl]amide;
5′-carboxy-2′,3-dichloro-biphenyl-4-carboxylic acid[1(S)-(cyanomethylcarbamoyl)-3-(methyl)butyl]amide;
4′-carboxy-2′,3-dichloro-biphenyl-4-carboxylic acid[1(S)-(cyanomethylcarbamoyl)-3-(methyl)butyl]amide;
5′-carboxy-2′,3-dichloro-biphenyl-4-carboxylic acid[1(S)-(cyanomethylcarbamoyl)-2-(2,6-difluoro-4-methoxyphenyl)ethyl]amide;
2′,3-dichloro-biphenyl-4-carboxylic acid[1(S)-(cyanomethylcarbamoyl)-3-(2S-(2-methoxyphenyl))butyl]amide;
2′,3-dichloro-biphenyl-4-carboxylic acid[1(S)-(cyanomethylcarbamoyl)-methyl]amide;
2′,3-dichloro-biphenyl-4-carboxylic acid[1(S)-(cyanomethylcarbamoyl)-ethyl]amide;
2′,3-dichloro-biphenyl-4-carboxylic acid[1(S)-(cyanomethylcarbamoyl)-2-methylpropyl]amide;75
1-methyl-3-trifluoromethyl-1H-thieno[2,3-c]pyrazolyl-5-carboxylic acid[1(S)-(cyanomethylcarbamoyl)-3-(2S-phenyl)butyl]amide;
5′-(methoxycarbonyl)-2′-chloro-biphenyl-4-carboxylic acid[1(S)-(cyanomethylcarbamoyl)-2-(2,6-difluoro-4-methoxyphenyl)ethyl]amide;
biphenyl-4-carboxylic acid[1(S)-(cyanomethylcarbamoyl)-3-(2S-(2-methoxyphenyl))butyl]amide;
4′-carboxybiphenyl-4-carboxylic acid[1(S)-(cyanomethylcarbamoyl)-3-(2S-(2-methoxyphenyl))butyl]amide;
4′-(methoxycarbonyl)-2′-chloro-biphenyl-4-carboxylic acid[1(S)-(cyanomethylcarbamoyl)-(2,6-difluorophenyl)ethyl]amide;
2-[4-(methoxycarbonyl)-2-chloro-phenyl]pyridinyl-5-carboxylic acid[1(S)-(cyanomethylcarbamoyl)-3-(methyl)butyl]amide;
5′-(methoxycarbonyl)-2′-chloro-biphenyl-4-carboxylic acid[1(S)-(cyanomethylcarbamoyl)-3-(2S-phenyl)butyl]amide;
2,2′,6′-trichlorobiphenyl-4-carboxylic acid[1(S)-(cyanomethylcarbamoyl)-3-(methyl)butyl]amide;
2,2′-dichlorobiphenyl-4-carboxylic acid[1(S)-(cyanomethylcarbamoyl)-3-(methyl)butyl]amide;
5-chloro-4-(2-chlorophenyl)thienyl-2-carboxylic acid[1(S)-(cyanomethylcarbamoyl)-3-(methyl)butyl]amide;
5-chloro-4-phenylthienyl-2-carboxylic acid[1(S)-(cyanomethylcarbamoyl)-3-(methyl)butyl]amide;
2-(2,5-dimethylphenyl)pyridinyl-5-carboxylic acid[1(S)-(cyanomethylcarbamoyl)-3-(methyl)butyl]amide;
2′-chloro-biphenyl-4-carboxylic acid[1(S)-(cyanomethylcarbamoyl)-3-(cyclohexyl)butyl]amide;
2′-chloro-biphenyl-4-carboxylic acid[1(S)-(cyanomethylcarbamoyl)-2-(1-methylindol-3-yl)ethyl]amide;
3-[5-methyl(thien-2-yl)]phenyl-1-carboxylic acid[1(S)-(cyanomethylcarbamoyl)-3-(methyl)butyl]amide;
1-methyl-3-phenyl-1H-thieno[2,3-c]pyrazolyl-5-carboxylic acid[1(S)-(cyanomethylcarbamoyl)-3-(2S-phenyl)butyl]amide; and

1,3-dimethyl-1H-thieno[2,3-c]pyrazolyl-5-carboxylic acid[1(S)-(cyanomethylcarbamoyl)-3-(2S-phenyl)butyl]amide.

TABLE 2

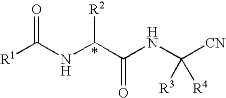

| Cpd. | | Stereochem. | | | |
|---|---|---|---|---|---|
| # | Mass Spec. | at (C*) | R¹ | R² | R³ + R⁴ |
| 1 | | (S) | 2′-Cl-biphen-4-yl | 2,6-diF-benzyl | tetrahydropyran-4-yl |
| 2 | 551.3 | (S) | 2′-Cl-biphen-4-yl | 2,6-diF-benzyl | N-ethylpiperidin-4-yl |
| | (M + 1) | | | | |
| 3 | | (S) | 2′-Cl-biphenyl-4-yl | 2,6-diF-benzyl | N-2,2,2-trifluoroethyl-piperidin-4-yl |
| 4 | | (S) | 2′-Cl-biphen-4-yl | 2,6-diF-benzyl | N-cyclopropylpiperidin-4-yl |
| 5 | 572.2 | (S) | 2′-Cl-biphen-4-yl | 2,6-diF-benzyl | 1,1-dioxotetrahydrothiopyran-4-yl |
| | (M + 1) | | | | |
| 6 | | (S) | 2-(2-Clphenyl)-pyridin-5-yl | 2,6-diF-benzyl | N-ethylpiperidin-4-yl |
| 7 | | (S) | 2-(2-Clphenyl)-pyridin-5-yl | 2,6-diF-benzyl | 1,1-dioxotetrahydrothiopyran-4-yl |
| 8 | | (S) | 2′,3-diCl-biphen-4-yl | 2,6-diF-benzyl | N-2,2,2-trifluoroethyl-piperidin-4-yl |
| 9 | | (S) | 2′,3-diCl-biphen-4-yl | 2,6-diF-benzyl | 1,1-dioxotetrahydrothiopyran-4-yl |
| 10 | 595 (M + 1) | (S) | 5′-carboxy-2′-chloro-biphenyl-4-yl | 2,6-diF-benzyl | N-ethylpiperidin-4-yl |
| 11 | | (S) | 2-(2-Clphenyl)-pyridin-5-yl | 2,6-diF-benzyl | tetrahydrothiopyran-4-yl |
| 12 | 587 (M + 1) | (S) | 5′-carboxy-2′-chloro-biphenyl-4-yl | 2S-phenylpropyl | N-ethylpiperidin-4-yl |
| 13 | | (S) | 2′-Cl-biphen-4-yl | 2S-phenylpropyl | 1,1-dioxotetrahydrothiopyran-4-yl |
| 14 | | (S) | 2′-Cl-biphen-4-yl | 2S-phenylpropyl | N-ethylpiperidin-4-yl |
| 15 | | (S) | 5′-carboxy-2′-chloro-biphenyl-4-yl | 2,6-diF-benzyl | tetrahydropyran-4-yl |
| 16 | | (S) | 2′,3-diCl-biphen-4-yl | 2,6-difluorophenyl-methyl | tetrahydrothiopyran-4-yl |
| 17 | | (S) | 2′-Cl-biphen-4-yl | 2(S)-phenylpropyl | tetrahydrothiopyran-4-yl |
| 18 | | (S) | 2′,3-diCl-biphen-4-yl | 2,6-diF-benzyl | tetrahydropyran-4-yl |
| 19 | | (S) | 2′,3-diCl-biphen-4-yl | 2,6-diF-benzyl | N-ethylpiperidin-4-yl |
| 20 | 543 (M + 1) | (S) | 2-(2-chlorophenyl)pyridin-5-yl | 2(S)-phenylpropyl | N-ethylpiperidin-4-yl |
| 21 | | (S) | 2-(2-chlorophenyl)-1-oxo-pyridin-5-yl | 2,6-diF-benzyl | 1,1-dioxo-tetrahydrothiopyran-4-yl |
| 22 | | (S) | 2′,3-diCl-biphen-4-yl | 2,6-diF-benzyl | N-cyclopropylpiperidin-4-yl |
| 23 | | (S) | 5′-carboxy-2′-Cl-biphen-4-yl | 2,6-diF-benzyl | tetrahydropyran-4-yl |
| 24 | | (S) | 2-(2-chlorophenyl)-pyridin-5-yl | 2(S)-phenylpropyl | tetrahydrothiopyran-4-yl |
| 25 | | (S) | 2-(2,6-dichlorophenyl)-pyridin-5-yl | 2,6-diF-benzyl | N-ethylpiperidin-4-yl |
| 26 | | (S) | 5′-carboxy-2′-Cl-biphen-4-yl | 2(S)-phenylpropyl | tetrahydrothiopyran-4-yl |
| 27 | 608 (M + 1) | (S) | 5′-carboxy-2′-Cl-biphen-4-yl | 2(S)-phenylpropyl | 1,1-dioxo-tetrahydrothiopyran-4-yl |
| 28 | | (S) | 5′-carboxy-2′-Cl-biphen-4-yl | 2,6-diF-benzyl | tetrahydrothiopyran-4-yl |
| 29 | | (S) | 2-(2-chlorophenyl)-pyridin-5-yl | 2(S)-phenylpropyl | tetrahydropyran-4-yl |
| 30 | 565 (M + 1) | (S) | 2-(2-chlorophenyl)-pyridin-5-yl | 2(S)-phenylpropyl | 1,1-dioxo-tetrahydrothiopyran-4-yl |
| 31 | 616 (M + 1) | (S) | 5′-carboxy-2′-Cl-biphen-4-yl | 2,6-diF-benzyl | 1,1-dioxo-tetrahydrothiopyran-4-yl |
| 32 | | (S) | 5′-carboxy-2′-Cl-biphen-4-yl | 2(S)-phenylpropyl | tetrahydropyran-4-yl |
| 33 | | (S) | 2′-Cl-biphen-4-yl | (2,6-difluoro-4-meth- | N-ethylpiperidin-4-yl |
| | | | | oxyphenyl)methyl | |
| 34 | | (S) | 2′-Cl-biphen-4-yl | 2(S)-phenylpropyl | tetrahydropyran-4-yl |
| 35 | | (S) | 2′-Cl-biphen-4-yl | 2(S)-phenylpropyl | N-(1,1,1-trifluoroeth-2-yl)piperidin-4-yl |
| 36 | | (S) | 2-(2-chlorophenyl)-1-oxo-pyridin-5-yl | 2(S)-phenylpropyl | tetrahydropyran-4-yl |
| 37 | 625 (M + 1) | (S) | 5′-carboxy-2′-Cl-biphen-4-yl | (2,6-difluoro-4-meth- | N-ethylpiperidin-4-yl |
| | | | | oxyphenyl)methyl | |
| 38 | | (S) | 2-(2-chlorophenyl)-pyridin-5-yl | (2,6-difluoro-4-meth- | N-ethylpiperidin-4-yl |
| | | | | oxyphenyl)methyl | |
| 39 | | (S) | 5′-carboxy-2′-Cl-biphen-4-yl | 2(S)-phenylpropyl | N-(1,1,1-trifluoroeth-2-yl)piperidin-4-yl |
| 40 | | (S) | 5′-(methoxycarbonyl)-2′-Cl-biphen-4-yl | 2(S)-phenylpropyl | N-(1,1,1-trifluoroeth-2-yl)piperidin-4-yl |
| 41 | | (S) | 5′-(methoxycarbonyl)-2′-Cl-biphen-4-yl | (2,6-difluoro-4-meth- | N-ethylpiperidin-4-yl |
| | | | | oxyphenyl)methyl | |
| 42 | | (S) | 5′-(methoxycarbonyl)-2′-Cl-biphen-4-yl | 2(S)-phenylpropyl | tetrahydropyran-4-yl |
| 43 | | (S) | 5′-(methoxycarbonyl)-2′-Cl-biphen-4-yl | (2,6-diF-phenyl)-methyl | tetrahydrothiopyran-4-yl |
| 44 | | (S) | 5′-(methoxycarbonyl)-2′-Cl-biphen-4-yl | 2(S)-phenylpropyl | tetrahydrothiopyran-4-yl |
| 45 | 601 (M + 1) | (S) | 5′-(methoxycarbonyl)-2′-Cl-biphen-4-yl | 2(S)-phenylpropyl | N-ethylpiperidin-4-yl |
| 46 | 609 (M + 1) | (S) | 5′-(methoxycarbonyl)-2′-Cl-biphen-4-yl | 2,6-diF-phenylmethyl | N-ethylpiperidin-4-yl |

and are named as:
2′-chlorobiphenyl-4-carboxylic acid[1(S)-[4-cyano-tetrahydropyran-4-ylcarbamoyl]-(2,6-difluorophenyl)ethyl]amide;
2′-chlorobiphenyl-4-carboxylic acid[1(S)-[4-cyano-1-ethylpiperidin-4-ylcarbamoyl]-(2,6-difluorophenyl)ethyl]amide;
2′-chlorobiphenyl-4-carboxylic acid[1(S)-[4-cyano-1-(2,2,2-trifluoroethyl)-piperidin-4-ylcarbamoyl]-(2,6-enyl)ethyl]amide;
2′-chlorobiphenyl-4-carboxylic acid[1(S)-[4-cyano-1-cyclopropylpiperidin-4-ylcarbamoyl]-(2,6-difluorophenyl)ethyl]amide;
2′-chlorobiphenyl-4-carboxylic acid[1(S)-[4-cyano-1,1-dioxotetrahydrothiopyran-4-ylcarbamoyl]-(2,6-difluorophenyl)ethyl]amide;
2-(2-chlorophenyl)pyridinyl-5-carboxylic acid[1(S)-[4-cyano-1-ethylpiperidin-4-ylcarbamoyl]-(2,6-difluorophenyl)ethyl]amide;
2-(2-chlorophenyl)pyridinyl-5-carboxylic acid[1(S)-[4-cyano-1,1-dioxotetrahydrothiopyran-4-ylcarbamoyl]-(2,6-difluorophenyl)ethyl]amide;
2′,3-dichlorobiphenyl-4-carboxylic acid[1(S)-[4-cyano-1-(2,2,2-trifluoroethyl)-piperidin-4-ylcarbamoyl]-(2,6-difluorophenyl)ethyl]amide;
2′,3-dichlorobiphenyl-4-carboxylic acid[1(S)-[4-cyano-1,1-dioxotetrahydrothiopyran-4-ylcarbamoyl]-(2,6-difluorophenyl)ethyl]amide;
5′-carboxy-2′-chlorobiphenyl-4-carboxylic acid[1(S)-[4-cyano-1-ethylpiperidin-4-ylcarbamoyl]-(2,6-difluorophenyl)ethyl]amide;
2-(2-chlorophenyl)pyridinyl-5-carboxylic acid[1(S)-[4-cyano-tetrahydrothiopyran-4-ylcarbamoyl]-(2,6-difluorophenyl)ethyl]amide;
2-chloro-4′-[1(S)-(4-cyano-1-ethylpiperidin-4-ylcarbanoyl)-3(S)-phenylbutyl-carbamoyl]biphenyl-5-carboxylic acid;
2′-chlorobiphenyl-4-carboxylic acid[1(S)-(4-cyano-1,1-dioxotetrahydrothiopyran-4-ylcarbamoyl)-3(S)-phenylbutyl]amide;
2′-chlorobiphenyl-4-carboxylic acid[1(S)-[4-cyano-1-ethylpiperidin-4-ylcarbamoyl]-3(S)-phenylbutyl]amide;
5′-methoxycarbonyl-2′-chlorobiphenyl-4-carboxylic acid[1(S)-[4-cyano-tetrahydropyran-4-ylcarbamoyl]-(2,6-difluorophenyl)ethyl]amide;
2′,3-dichlorobiphenyl-4-carboxylic acid[1(S)-[4-cyano-tetrahydrothiopyran-4-ylcarbamoyl]-(2,6-difluorophenyl)ethyl]amide;
2′-chlorobiphenyl-4-carboxylic acid[1(S)-[4-cyano-tetrahydrothiopyran-4-ylcarbamoyl]-3(S)-phenylbutyl]amide;
2′,3-dichlorobiphenyl-4-carboxylic acid[1(S)-[4-cyano-tetrahydropyran-4-ylcarbamoyl]-(2,6-difluorophenyl)ethyl]amide;
2′,3-dichlorobiphenyl-4-carboxylic acid[1(S)-[4-cyano-1-ethylpiperidin-4-ylcarbamoyl]-(2,6-fluorophenyl)ethyl]amide;
2-(2-chlorophenyl)pyridinyl-5-carboxylic acid[1(S)-[4-cyano-1-ethylpiperidin-4-ylcarbamoyl]-3(S)-phenylbutyl]amide;
2-(2-chlorophenyl)pyridinyl-5-carboxylic acid[1(S)-[4-cyano-1,1-dioxotetrahydrothiopyran-4-ylcarbamoyl]-(2,6-difluorophenyl)ethyl]amide;
2′,3-dichlorobiphenyl-4-carboxylic acid[1(S)-[4-cyano-1-cyclopropylpiperidin-4-ylcarbamoyl]-(2,6-difluorophenyl)ethyl]amide;
5′-carboxy-2′-chlorobiphenyl-4-carboxylic acid[1(S)-[4-cyano-tetrahydropyran-4-ylcarbamoyl]-(2,6-difluorophenyl)ethyl]amide;
2-(2-chlorophenyl)pyridinyl-5-carboxylic acid[1(S)-[4-cyano-tetrahydrothiopyran-4-ylcarbamoyl]-3(S)-phenylbutyl]amide;
2-2,6-dichlorophenyl)pyridinyl-5-carboxylic acid[1(S)-[4-cyano-1-ethylpiperidin-4-ylcarbamoyl]-(2,6-difluorophenyl)ethyl]amide;
5′-carboxy-2′-chlorobiphenyl-4-carboxylic acid[1(S)-[4-cyano-tetrahydrothiopyran-4-ylcarbamoyl]-3(S)-phenylbutyl]amide;
5′-carboxy-2′-chlorobiphenyl-4-carboxylic acid[1(S)-[4-cyano-1,1-dioxotetrahydrothiopyran-4-ylcarbamoyl]-3(S)-phenylbutyl]amide;
5′-carboxy-2′-chlorobiphenyl-4-carboxylic acid[1(S)-[4-cyano-tetrahydrothiopyran-4-ylcarbamoyl]-(2,6-difluorophenyl)ethyl]amide;
2-(2-chlorophenyl)pyridinyl-5-carboxylic acid[1(S)-[4-cyano-tetrahydropyran-4-ylcarbamoyl]-3(S)-phenylbutyl]amide;
2-(2-chlorophenyl)pyridinyl-5-carboxylic acid[1(S)-[4-cyano-1,1-dioxotetrahydrothiopyran-4-ylcarbamoyl]-3(S)-phenylbutyl]amide;
5′-carboxy-2′-chlorobiphenyl-4-carboxylic acid[1 (S)-[4-cyano-1,1-dioxotetrahydrothiopyran-4-ylcarbamoyl]-(2,6-difluorophenyl)ethyl]amide;
5′-carboxy-2′-chlorobiphenyl-4-carboxylic acid[1(S)-[4-cyano-tetrahydropyran-4-ylcarbamoyl]-3(S)-phenylbutyl]amide;
2′-chlorobiphenyl-4-carboxylic acid[1(S)-[4-cyano-1-ethylpiperidin-4-ylcarbamoyl]-(2,6-difluoro-4-methoxyphenyl)ethyl]amide;
2′-chlorobiphenyl-4-carboxylic acid[1(S)-[4-cyano-tetrahydropyran-4-ylcarbamoyl]-3(S)-phenylbutyl]amide;
2′-chlorobiphenylcarboxylic acid[1(S)-[4-cyano-1-(2,2,2-trifluoroethyl)-piperidin-4-ylcarbamoyl]-3(S)-phenylbutyl]amide;
2-(2-chlorophenyl)-1-oxo-pyridinyl-5-carboxylic acid[1(S)-[4-cyano-tetrahydropyran-4-ylcarbamoyl]-3(S)-phenylbutyl]amide;
5′-carboxy-2′-chlorobiphenyl-4-carboxylic acid[1(S)-[4-cyano-1-ethylpiperidin-4-ylcarbamoyl]-(2,6-difluoro-4-methoxyphenyl)ethyl]amide;
2-(2-chlorophenyl)pyridinyl-5-carboxylic acid[1(S)-[4-cyano-1-ethylpiperidin-4-ylcarbamoyl]-(2,6-difluoro-4-methoxyphenyl)ethyl]amide;
5-carboxy-2′-chlorobiphenyl-4-carboxylic acid[1(S)-[4-cyano-1-(2,2,2-trifluoroethyl)-piperidin-4-ylcarbamoyl]-3(S)-phenylbutyl]amide;
5′-methoxycarbonyl-2′-chlorobiphenyl-4-carboxylic acid[1(S)-[4-cyano-1-(2,2,2-trifluoroethyl)-piperidin-4-ylcarbamoyl]-3(S)-phenylbutyl]amide;
5′-methoxycarbonyl-2′-chlorobiphenyl-4-carboxylic acid[1(S)-[4-cyano-1-ethylpiperidin-4-ylcarbamoyl]-(2,6-difluoro-4-methoxyphenyl)ethyl]amide;
5′-methoxycarbonyl-2′-chlorobiphenyl-4-carboxylic acid[1(S)-[4-cyano-tetrahydropyran-4-ylcarbamoyl]-3(S)-phenylbutyl]amide;
5′-methoxycarbonyl-2′-chlorobiphenyl-4-carboxylic acid[1(S)-[4-cyano-tetrahydrothiopyran-4-ylcarbamoyl]-(2,6-difluorophenyl)ethyl]amide;
5′-methoxycarbonyl-2′-chlorobiphenyl-4-carboxylic acid[1(S)-[4-cyano-tetrahydrothiopyran-4-ylcarbamoyl]-3(S)-phenylbutyl]amide;
5′-methoxycarbonyl-2′-chlorobiphenyl-4-carboxylic acid[1(S)-[4-cyano-1-ethylpiperidin-4-ylcarbamoyl]-3(S)-phenylbutyl]amide; and

5′-methoxycarbonyl-2′-chlorobiphenyl-4-carboxylic acid[1(S)-[4-cyano-1-ethylpiperidin-4-ylcarbamoyl]-(2,6-difluorophenyl)ethyl]amide.

TABLE 3

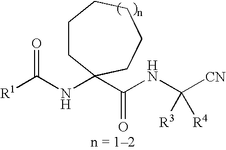

| Cpd. # | Mass Spec. | n | R¹ | R³ | R⁴ |
|---|---|---|---|---|---|
| 1 | 443 (M − 1) | 1 | 3,2′-diCl-biphen-4-yl | H | H |
| 2 | | 1 | 2-(2,6-dichlorophenyl)pyridin-5-yl | H | H |
| 3 | | 2 | 2′-Cl-biphen-4-yl | H | H |
| 4 | | 1 | 2-[2-chloro-5-(tert-butoxy- | H | H |
| | | | carbonyl)phenyl]pyridin-5-yl | | |

and are named as
2′-dichlorobiphenyl-4-carboxylic acid[1-(cyanomethylcarbamoyl)-cycloheptyl]amide;
2-(2,6-dischlorophenyl)pyridinyl-5-carboxylic acid[1-(cyanomethylcarbamoyl)-cycloheptyl]amide;
2′-chlorobiphenyl-4-carboxylic acid[1-(cyanomethylcarbamoyl)-cyclooctyl]amide; and

2-[2-chloro-5-(tert-butoxycarbonyl)phenyl]pyridinyl-5-carboxylic acid[1-(cyanomethylcarbamoyl)-cycloheptyl]amide.

TABLE 4

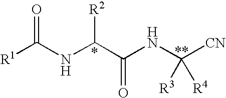

| | Stereochem. | | | | |
|---|---|---|---|---|---|
| Cpd. # | at (C*/C**) | R¹ | R² | R³ | R⁴ |
| | (RS)/(RS) | 2′-Cl-bi- | 2,6-diF-phenyl- | H | 3-methyl- |
| | | phen-4-yl | methyl | | thien-2-yl |
| | (RS)/(RS) | 2′-Cl-bi- | 2,6-diF-phenyl- | H | 5-methyl- |
| | | phen-4-yl | methyl | | furan-2-yl |

and are named as
2′-chlorobiphenyl-4-carboxylic acid[1(S)-[1-cyano-1-(3-methyl-thien-2-yl)methyl-carbamoyl]-(2,6-fluorophenyl)ethyl]amide; and
2′-chlorobiphenyl-4-carboxylic acid[1(S)-[1-cyano-1-(5-methylfuran-2-yl)methyl-carbamoyl]-(2,6-difluorophenyl)ethyl]amide.

### PREFERRED EMBODIMENTS

While the broadest definition of this invention is set forth in the Summary of the Invention, certain compounds of Formula (I) are preferred.

I. A preferred group of compounds is represented by Formula (Ia). Within this group:

A. One preferred group of compounds is that wherein R³, R⁴ and R⁵ are hydrogen and R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino, preferably the phenyl ring in the benzyl group is substituted at the 2 and 6 positions with groups independently selected from methyl, chloro, fluoro, trifluoromethyl, methoxy, trifluoromethoxy, or difluoromethoxy and at the 4 position with hydrogen, methyl, ethyl, propyl, chloro, fluoro, trifluoromethyl, methoxy, 2-methoxyethyloxy, 2-dimethylaminoethyloxy, trifluoromethoxy, difluoromethoxy, methylthio, methylsulfinyl, methylsulfonyl, cyano, amino, methylamino or dimethylamino. More preferably, R² is cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl, and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, haloalkoxy, or alkylthio.

B. Another preferred group of compounds is that wherein R³ is hydrogen and R⁴ and R⁵ together with the carbon atom to which they are attached form cycloalkylene, preferably cyclopropylene, and R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino, preferably the phenyl ring in the benzyl group is substituted at the 2 and 6 positions with groups independently selected from methyl, chloro, fluoro, trifluoromethyl, methoxy, trifluoromethoxy, or difluoromethoxy and at the 4 position with hydrogen, methyl, ethyl, propyl, chloro, fluoro, trifluoromethyl, methoxy, 2-methoxyethyloxy, 2-dimethylaminoethyloxy, trifluoromethoxy, difluoromethoxy, methylthio, methylsulfinyl, methylsulfonyl, cyano, amino, methylamino or dimethylamino. More preferably, R² is cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl, and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, haloalkoxy, or alkylthio.

C. Yet another preferred group of compounds is that wherein R³ is hydrogen, R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino, preferably the phenyl ring in the benzyl group is substituted at the 2 and 6 positions with groups independently selected from methyl, chloro, fluoro, trifluoromethyl, methoxy, trifluoromethoxy, or difluoromethoxy and at the 4 position with hydrogen, methyl, ethyl, propyl, chloro, fluoro, trifluoromethyl, methoxy, 2-methoxyethyloxy, 2-dimethylaminoethyloxy, trifluoromethoxy, difluoromethoxy, methylthio, methylsulfinyl, methylsulfonyl, cyano, amino, methylamino or dimethylamino; and R⁴ and R⁵ together with the carbon atom to which they are attached form heterocycloalkylene, preferably, R⁴ and R⁵ together with the carbon atom to which they are attached form: 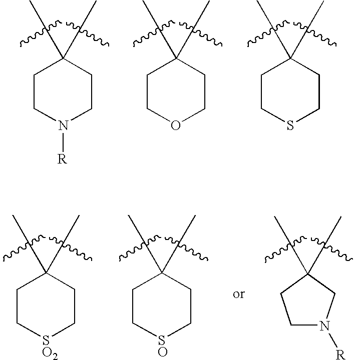
wherein R is hydrogen, alkyl, haloalkyl or cycloalkyl, preferably methyl, ethyl, 2,2,2-trifluoroethyl, or cyclopropyl. More preferably, R² is cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl, and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, haloalkoxy, or alkylthio.
D. Yet another preferred group of compounds is that wherein R², R³, R⁴ and R⁵ are hydrogen.
E. Yet preferred group of compounds is that wherein R³ and R⁴ are hydrogen, R⁵ is heteroaryl optionally substituted with alkyl, haloalkyl, halo, haloalkoxy, or alkoxy, preferably 3-methylthien-2-yl or 5-methylfuran-2-yl, and R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino, preferably the phenyl ring in the benzyl group is substituted at the 2 and 6 positions with groups independently selected from methyl, chloro, fluoro, trifluoromethyl, methoxy, trifluoromethoxy, or difluoromethoxy and at the 4 position with hydrogen, methyl, ethyl, propyl, chloro, fluoro, trifluoromethyl, methoxy, 2-methoxyethyloxy, 2-dimethylaminoethyloxy, trifluoromethoxy, difluoromethoxy, methylthio, methylsulfinyl, methylsulfonyl, cyano, amino, methylamino or dimethylamino. More preferably, R² is cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl, and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, haloalkoxy, or alkylthio.
F. Yet another preferred group of compounds is that wherein R² is hydrogen, methyl, or 2-propyl.
1. Within the above groups (A-F), a more preferred group of compounds is that wherein R¹ is: 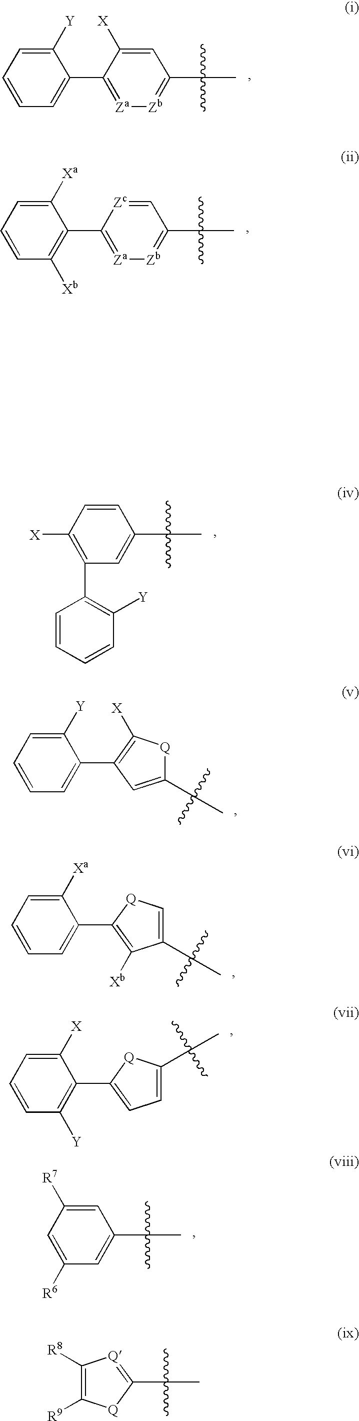 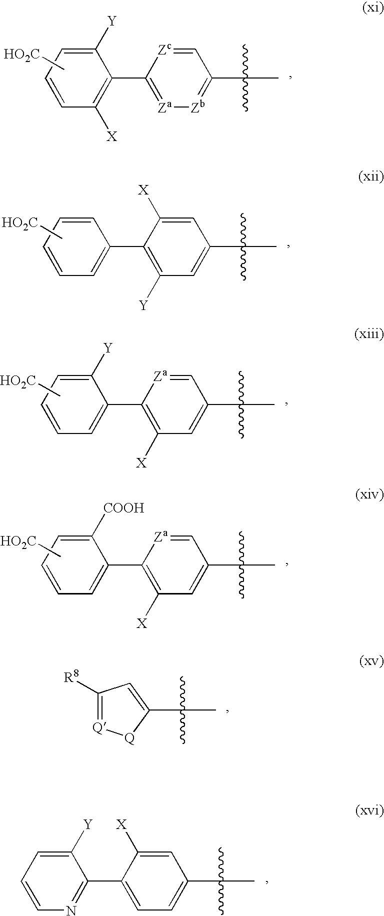
(xvii) 1-(4-aminosulfonylphenyl)-5-(4-chlorophenyl)pyrazol-3-yl;
(xix) 4-(3,5-dimethyloxazol-4-yl)phenyl; or
(xx) 4-(5-carboxy-2-methylthiophen-3-yl)phenyl; wherein:

X is hydrogen, chloro, methyl, methoxy, trifluoromethyl, or trifluoromethoxy, preferably hydrogen, chloro, methyl, methoxy, or trifluoromethoxy, more preferably hydrogen, chloro, methyl, or methoxy;

Y is chloro, methyl, methoxy, trifluoromethyl, or trifluoromethoxy, preferably chloro, methyl, methoxy, or trifluoromethoxy, more preferably chloro, methyl, or methoxy;

X^{a}, and X^{b} are independently selected from methyl, chloro, fluoro, methoxy, trifluoromethyl, or trifluoromethoxy, preferably methyl, chloro, fluoro, methoxy, or trifluoromethoxy, more preferably chloro, methyl, or methoxy;

R⁶ is phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl;

R⁷ is 2-alkoxyphenyl, 3-alkoxyphenyl, 2-halophenyl, 2-alkylphenyl, 2-haloalkylphenyl, or 2-haloalkoxyphenyl;

R⁸ is 2-alkoxyphenyl, 3-alkoxyphenyl, 2-halophenyl, 2-alkylphenyl, 2-haloalkylphenyl, or 2-haloalkoxyphenyl;

R⁹ is phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl.

Within the above preferred and more preferred groups, an even more preferred group of compounds is that wherein R² is preferably selected from the group consisting of cyclohexyl, cycloheptyl, thiazol-2-ylmethyl, 2-ethylbutyl, pyrazol-1-ylmethyl, 2-methylpropyl, 2,4,4-trimethylpentyl, 2-napth-1-ylpropyl, 2-phenylprop-2-enyl, 2-phenyl-2-methylpropyl, 2-phenylpropyl, 2-(2-methoxyphenyl)propyl, 4-methylindol-3-ylmethyl, 2-(2,5-dimethylphenyl)propyl, benzyloxymethyl, 2-(2,4-dimethylphenyl)propyl, 2-(2,4-dichlorophenyl)-propyl, 2,6-difluorobenzyl, 2,5-difluorobenzyl, 2,6-difluoro-4-methoxybenzyl, 2-methyl-2-(2-methoxyphenyl)propyl, and 2,3-difluorobenzyl. More preferably, R² is 2,6-difluorobenzyl, 2,6-difluoro-4-methoxybenzyl, 2(S)-phenylpropyl, 2(R)-phenylpropyl, 2-methylpropyl, 2-methyl-2-phenylpropyl, 2-phenylethyl, 2-phenylprop-2-enyl, benzyl, or thiazol-2-ylmethyl. Particularly preferably, R² is 2,6-difluorobenzyl, 2,6-difluoro-4-methoxybenzyl, or 2(S)-phenylpropyl and the stereochemistry at the carbon to which R² is attached is (S) when the Prelog rule places the order of the substituent 1) N, 2) —COOH, 3) R² and 4) H; and (R) when the Prelog rule places the order of the substituent 1) N, 2) R², 3) —COOH and 4) H.

Within the above preferred and more preferred and an even more preferred group of compounds, a particularly preferred group of compounds is that wherein R¹ is 2′-chlorobiphen-4-yl, 3,2′-dichlorobiphen-4-yl, 2′,6′-dichlorobiphen-4-yl, 2′,6′-dimethylbiphen-4-yl, 2′-methylbiphen-4-yl, 2′-fluorobiphen-4-yl; 2-(2-methylphenyl)furan-5-yl, 2-(2-methoxyphenyl)furan-5-yl, 3-methoxy-2-(2-methylphenyl)thiophen-4-yl, 3-methoxy-2-(2-methoxyphenyl)thiophen-4-yl, 2,3-di(2-methoxyphenyl)thiophen-5-yl, 3,5-di(2-methoxyphenyl)phenyl, 3,5-di(3-methoxyphenyl)phenyl, 2,3-di(2-methylphenyl)thiophen-5-yl, 4-(2-methylphenyl)thiophen-2-yl, 4-(2-methoxyphenyl)thiophen-2-yl, 2′-chlorobiphen-3-yl, 2′-methyl-4-chlorobiphen-3-yl, 3,5-di(2-chlorophenyl)phenyl, 2,3-di(2-chlorophenyl)thiophen-5-yl, 1-(4-aminosulfonylphenyl)-5-(4-chlorophenyl)pyrazol-3-yl, 2-(2,6-dichlorophenyl)furan-5-yl, 3-trifluoromethyl-1-methylthieno[2,3-c]pyrazol-5-yl, 2′-methoxybiphen-4-yl, 2′-trifluoromethylbiphen-4-yl, 2′-methyl-3-chlorobiphen-4-yl, 2-(2-chlorophenyl)pyridin-5-yl, 2-(2,6-dichlorophenyl)pyridin-5-yl, 2-(2-trifluoromethylphenyl)pyridin-5-yl, 4-(3-methylpyridin-2-yl)phenyl, 2-(2-chlorophenyl)-3-chloropyridin-5-yl, 2-(2,6-dichlorophenyl)-3-chloropyridin-5-yl, 4′-carboxy-2′-chlorobiphen-4-yl, 4′-carboxy-2′-fluorobiphen-4-yl, 4′-carboxy-2′-methylbiphen-4-yl, 5′-carboxy-2′-chlorobiphen-4-yl, 5′-carboxy-2′-methylbiphen-4-yl, 2-(4-carboxy-2-chlorophenyl)pyridin-5-yl, 2-(5-carboxy-2-chlorophenyl)pyridin-5-yl, 4-(5-carboxy-2-methylthiophen-3-yl)phenyl, 4-(3-methoxy-phenyl)thiophen-2-yl, 3-(2-chlorophenyl)isoxazol-5-yl, or 4-(3-methylpyridin-2-yl)phenyl. More preferably, R¹ is 2′-chlorobiphen-4-yl, 2′,6′-dichlorobiphen-4-yl, 2′,3-dichlorobiphen-4-yl, or 2-(2-chlorophenyl)pyridin-5-yl.

Within the above preferred and more preferred and an even more preferred group of compounds, another particularly preferred group of compounds is that wherein R¹ is 4-(2-chlorophenyl)thiophen-2-yl, 3-chloro-2′-methylbiphen-4-yl, 1-oxo-2-(2,6-dichlorophenyl)pyridin-5-yl, 1-oxo-2-(2-methylphenyl)pyridin-5-yl, 4′-carboxy-2′-methylbiphen-4-yl, 1-oxo-3-chloro-2-(2-chlorophenyl)pyridin-5-yl, 3-chloro-2-(2-trifluoromethylphenyl)pyridin-5-yl, 3-chloro-2-(2-methylphenyl)pyridin-5-yl, 1-oxo-2-(2-chlorophenyl)pyridin-5-yl, 4′-carboxy-2′6′-dichlorobiphen-4-yl, or 4′-carboxy-3,2′-dichlorobiphen-4-yl.

2. Within the above groups (A-F), another more preferred group of compounds is that wherein:

R¹ is a group of formula: 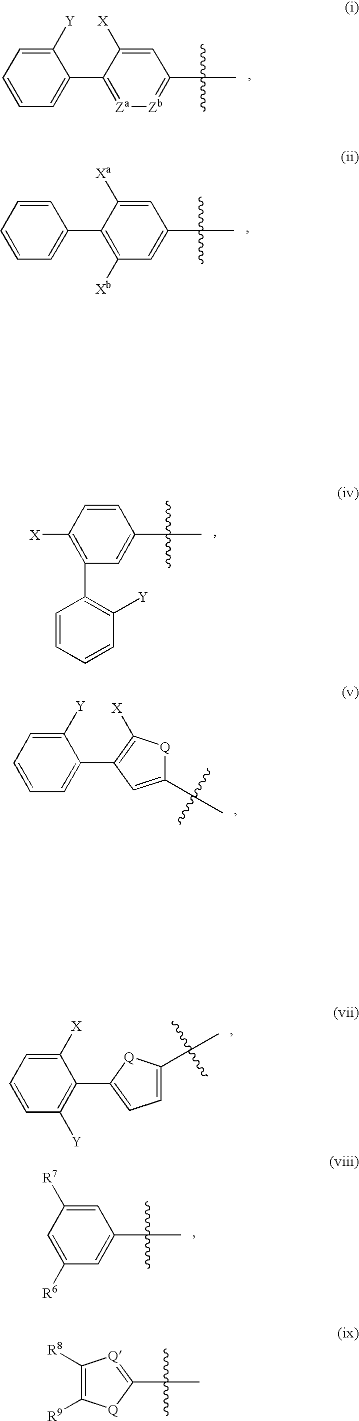 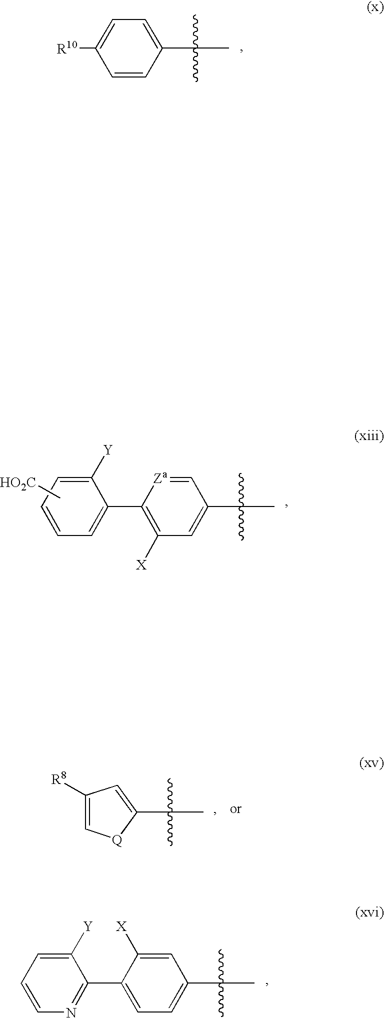
where:

X is chloro, methyl, methoxy, trifluoromethyl, or trifluoromethoxy, preferably chloro, methyl, methoxy, or trifluoromethoxy, more preferably hydrogen, chloro, methyl, or methoxy;

Y is hydrogen;

X^{a}, and X^{b} are independently selected from methyl, chloro, fluoro, methoxy, trifluoromethyl, or trifluoromethoxy, preferably methyl, chloro, fluoro, methoxy, or trifluoromethoxy, more preferably chloro, methyl, or methoxy.

R⁶ and R⁷ are phenyl;

R⁸ are independently selected from phenyl, furan-2-yl, thiophen-3-yl, or pyridin-4-yl;

R⁹ is phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl; and

R¹⁰ is a branched alkyl chain of 4-6 carbon atoms or trifluoroalkoxy.

Within the above preferred and more preferred groups, an even more preferred group of compounds is that wherein R² is preferably selected from the group consisting of cyclohexyl, cycloheptyl, thiazol-2-ylmethyl, 2-ethylbutyl, pyrazol-1-ylmethyl, 2-methylpropyl, 2,4,4-trimethylpentyl, 2-napth-1-ylpropyl, 2-phenylprop-2-enyl, 2-phenyl-2-methylpropyl, 2-phenylpropyl, 2-(2-methoxyphenyl)propyl, 4-methylindol-3-ylmethyl, 2-(2,5-dimethylphenyl)propyl, benzyloxymethyl, 2-(2,4-dimethylphenyl)propyl, 2-(2,4-dichlorophenyl)-propyl, 2,6-difluorobenzyl, 2,5-difluorobenzyl, 2-methyl-2-(2-methoxyphenyl)propyl, 2,6-difluoro-4-methoxybenzyl, and 2,3-difluorobenzyl; particularly preferably R² is 2,6-difluorobenzyl, 2,6-difluoro-4-methoxybenzyl, or 2S-phenylpropyl and the stereochemistry at the carbon to which R² is attached is (S) when the Prelog rule places the order of the substituent 1) N, 2) —COOH, 3) R² and 4) H; and (R) when the Prelog rule places the order of the substituent 1) N, 2) R², 3) —COOH and 4) H.

Within these preferred, more preferred groups, a particularly preferred group of compounds is that wherein R¹ is 4-trifluoromethoxyphenyl, 4-(2-butyl)phenyl, 3,5-diphenylphenyl, 2,3-diphenylthiophen-5-yl, 3,5-di(thiophen-3-yl)phenyl, 3,5-di(pyridin-4-yl)phenyl, 4-tert-butylphenyl, 2,3-di(furan-2-yl)thiophen-5-yl, 3,5-di(furan-2-yl)phenyl, 2,3-diphenylthiophen-5-yl, 4,5-diphenylthiazol-2-yl, 3-methylbiphen-4-yl.

G. Another preferred group of compounds is that wherein R² and R³ together with the carbon atom to which they are attached form cycloheptylene or cyclooctylene and R⁴ and R⁵ are hydrogen.

H. Another preferred group of compounds is that wherein R² and R³ together with the carbon atom to which they are attached form cycloheptylene or cyclooctylene and R⁴ and R⁵ together with the carbon atom to which they are attached form cycloalkylene, preferably cyclopropylene.
I. Yet another preferred group of compounds is that wherein R² and R³ together with the carbon atom to which they are attached form cycloheptylene or cyclooctylene and R⁴ and R⁵ together with the carbon atom to which they are attached form heterocycloalkylene, preferably, R⁴ and R⁵ together with the carbon atom to which they are attached form: 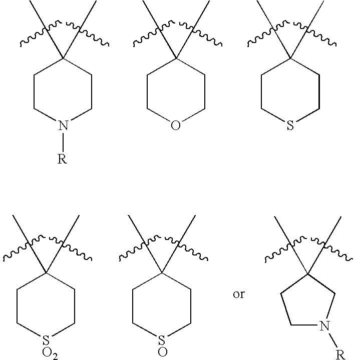
wherein R is hydrogen, alkyl, haloalkyl or cycloalkyl, preferably methyl, ethyl, 2,2,2-trifluoroethyl, or cyclopropyl.
3. Within the above groups (G-I), a more preferred group of compounds is that wherein R¹ is: 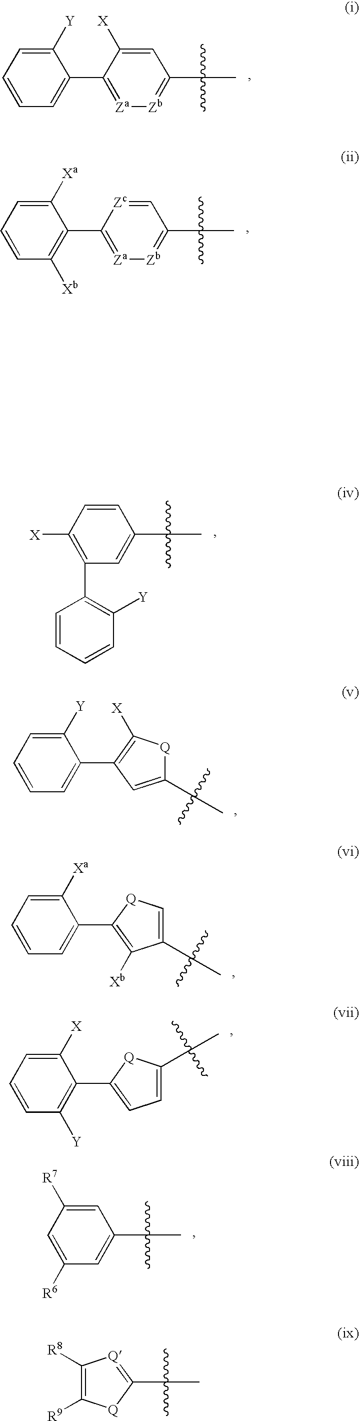 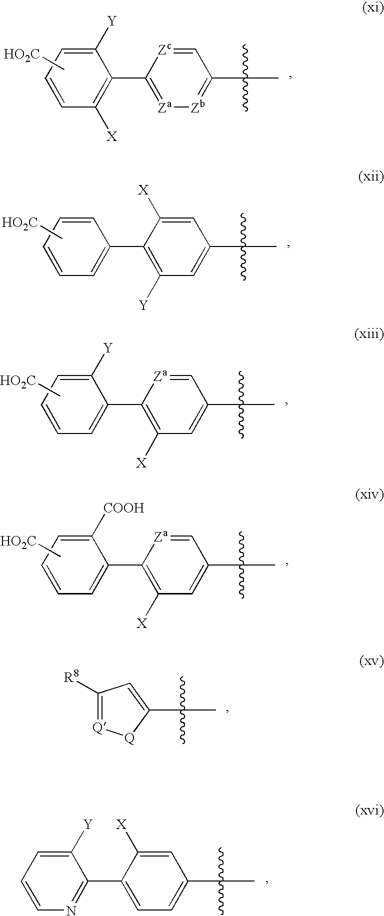
(xvii) 1-(4-aminosulfonylphenyl)-5-(4-chlorophenyl)pyrazol-3-yl;
(xix) 4-(3,5-dimethyloxazol-4-yl)phenyl; or
(xx) 4-(5-carboxy-2-methylthiophen-3-yl)phenyl;
wherein:

X is hydrogen, chloro, methyl, methoxy, trifluoromethyl, or trifluoromethoxy, preferably hydrogen, chloro, methyl, methoxy, or trifluoromethoxy, more preferably hydrogen, chloro, methyl, or methoxy;

Y is chloro, methyl, methoxy, trifluoromethyl, or trifluoromethoxy, preferably chloro, methyl, methoxy, or trifluoromethoxy, more preferably chloro, methyl, or methoxy;

X^{a}, and X^{b} are independently selected from methyl, chloro, fluoro, methoxy, trifluoromethyl, or trifluoromethoxy, preferably methyl, chloro, fluoro, methoxy, or trifluoromethoxy, more preferably chloro, methyl, or methoxy;

R⁶ is phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl;

R⁷ is 2-alkoxyphenyl, 3-alkoxyphenyl, 2-halophenyl, 2-alkylphenyl, 2-haloalkylphenyl, or 2-haloalkoxyphenyl;

R⁸ is 2-alkoxyphenyl, 3-alkoxyphenyl, 2-halophenyl, 2-alkylphenyl, 2-haloalkylphenyl, or 2-haloalkoxyphenyl;

R⁹ is phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl.

Within the above preferred and more preferred and an even more preferred group of compounds, a particularly preferred group of compounds is that wherein R¹ is 2′-chlorobiphen-4-yl, 3,2′-dichlorobiphen-4-yl, 2′,6′-dichlorobiphen-4-yl, 2′,6′-dimethylbiphen-4-yl, 2′-methylbiphen-4-yl, 2′-fluorobiphen-4-yl; 2-(2-methylphenyl)furan-5-yl, 2-(2-methoxyphenyl)furan-5-yl, 3-methoxy-2-(2-methylphenyl)thiophen-4-yl, 3-methoxy-2-(2-methoxyphenyl)thiophen-4-yl, 2,3-di(2-methoxyphenyl)thiophen-5-yl, 3,5-di(2-methoxyphenyl)phenyl, 3,5-di(3-methoxyphenyl)phenyl, 2,3-di(2-methylphenyl)thiophen-5-yl, 4-(2-methylphenyl)thiophen-2-yl, 4-(2-methoxyphenyl)thiophen-2-yl, 2′-chlorobiphen-3-yl, 2′-methyl-4-chlorobiphenyl-3-yl, 3,5-di(2-chlorophenyl)phenyl, 2,3-di(2-chlorophenyl)thiophen-5-yl, 1-(4-aminosulfonylphenyl)-5-(4-chlorophenyl)pyrazol-3-yl, 2-(2,6-dichlorophenyl)furan-5-yl, 3-trifluoromethyl-1-methylthieno[2,3-c]pyrazol-5-yl, 2′-methoxybiphen-4-yl, 2′-trifluoromethylbiphen-4-yl, 2′-methyl-3-chlorobiphen-4-yl, 2-(2-chlorophenyl)pyridin-5-yl, 2-(2,6-dichlorophenyl)pyridin-5-yl, 2-(2-trifluoromethylphenyl)pyridin-5-yl, 4-(3-methylpyridin-2-yl)phenyl, 2-(2-chlorophenyl)-3-chloropyridin-5-yl, 2-(2,6-dichlorophenyl)-3-chloropyridin-5-yl, 4′-carboxy-2′-chlorobiphen-4-yl, 4′-carboxy-2′-fluorobiphen-4-yl, 4′-carboxy-2′-methylbiphen-4-yl, 5′-carboxy-2′-chlorobiphen-4-yl, 5′-carboxy-2′-methylbiphen-4-yl, 2-(4-carboxy-2-chlorophenyl)pyridin-5-yl, 2-(5-carboxy-2-chlorophenyl)pyridin-5-yl, 4-(5-carboxy-2-methylthiophen-3-yl)phenyl, 4-(3-methoxy-phenyl)thiophen-2-yl, 3-(2-chlorophenyl)isoxazol-5-yl, or 4-(3-methylpyridin-2-yl)phenyl. More preferably, R¹ is 2′-chlorobiphen-4-yl, 2′,6′-dichlorobiphen-4-yl, 2′,3-dichlorobiphen-4-yl, or 2-(2-chlorophenyl)pyridin-5-yl.

Within the above preferred and more preferred and an even more preferred group of compounds, another particularly preferred group of compounds is that wherein R¹ is 4-(2-chlorophenyl)thiophen-2-yl, 3-chloro-2′-methylbiphen-4-yl, 1-oxo-2-(2,6-dichlorophenyl)pyridin-5-yl, 1-oxo-2-(2-methylphenyl)pyridin-5-yl, 4′-carboxy-2′-methylbiphen-4-yl, 1-oxo-3-chloro-2-(2-chlorophenyl)pyridin-5-yl, 3-chloro-2-(2-trifluoromethylphenyl)pyridin-5-yl, 3-chloro-2-(2-methylphenyl)pyridin-5-yl, 1-oxo-2-(2-chlorophenyl)pyridin-5-yl, 4′-carboxy-2′6′-dichlorobiphen-4-yl, or 4′-carboxy-3,2′-dichlorobiphen-4-yl.
4. Within the above groups (G-I), another more preferred group of compounds is that wherein R¹ is a group of formula:

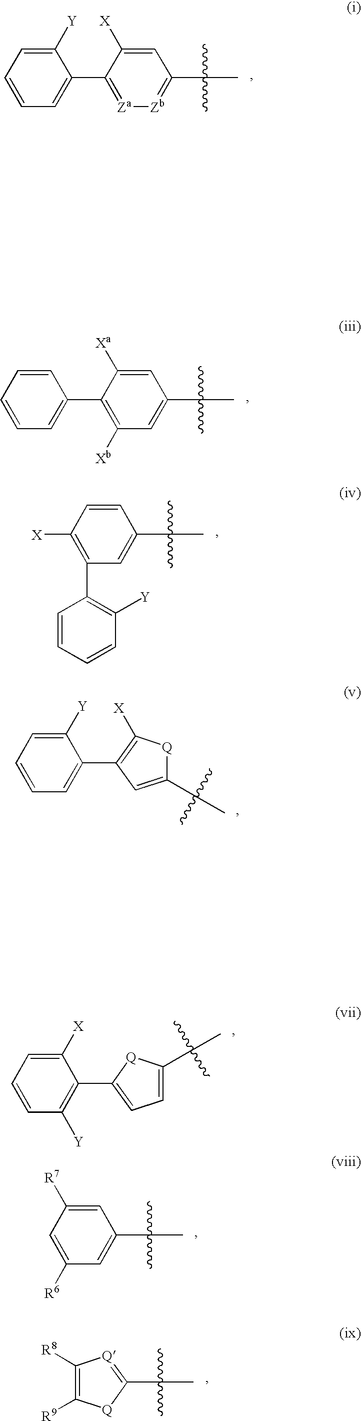

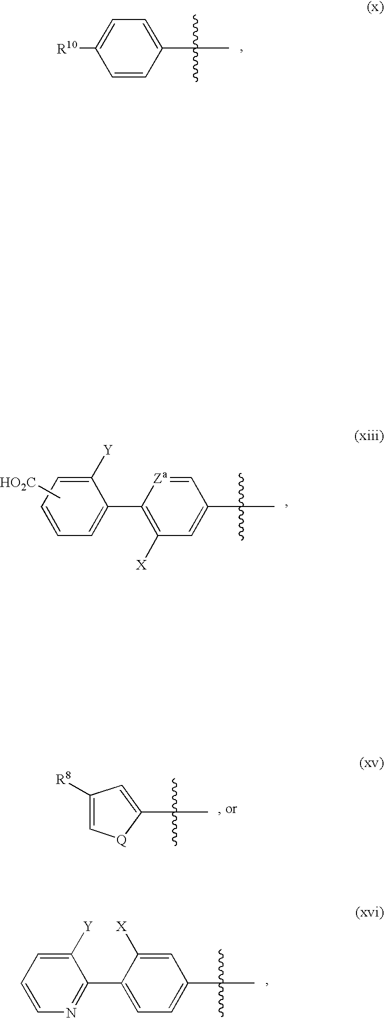

where:

X is chloro, methyl, methoxy, trifluoromethyl, or trifluoromethoxy, preferably chloro, methyl, methoxy, or trifluoromethoxy, more preferably hydrogen, chloro, methyl, or methoxy;

Y is hydrogen;

X^{a}, and X^{b} are independently selected from methyl, chloro, fluoro, methoxy, trifluoromethyl, or trifluoromethoxy, preferably methyl, chloro, fluoro, methoxy, or trifluoromethoxy, more preferably chloro, methyl, or methoxy.

R⁶ and R⁷ are phenyl;

R⁸ are independently selected from phenyl, furan-2-yl, thiophen-3-yl, or pyridin-4-yl;

R⁹ is phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl; and

R¹⁰ is a branched alkyl chain of 4-6 carbon atoms or trifluoroalkoxy.

Within these preferred, more preferred groups, a particularly preferred group of compounds is that wherein R¹ is 4-trifluoromethoxyphenyl, 4-(2-butyl)phenyl, 3,5-diphenylphenyl, 2,3-diphenylthiophen-5-yl, 3,5-di(thiophen-3-yl)phenyl, 3,5-di(pyridin-4-yl)phenyl, 4-tert-butylphenyl, 2,3-di(furan-2-yl)thiophen-5-yl, 3,5-di(furan-2-yl)phenyl, 2,3-diphenylthiophen-5-yl, 4,5-diphenylthiazol-2-yl, 3-methylbiphen-4-yl.

J. Yet another preferred groups of compounds are those wherein:

(i) R³, R⁴ and R⁵ are hydrogen; or

(ii) R³ is hydrogen and R⁴ and R⁵ together with the carbon atom to which they are attached form tetrahydropyran-4-yl, tetrahydrothiopyran-4-yl, N-ethylpiperidin-4-yl, N-2,2,2-trifluoroethylpiperidin-4-yl, N-cyclopropylpiperidin-4-yl, or 1,1-dioxotetrahydrothiopyran-4-yl;

R¹ is biphenyl, 4′-carboxybiphen-4-yl, 2′-Cl-biphen-4-yl, 2′,6′-dichlorobiphen-4-yl, 2-(2-Clphenyl)pyridin-5-yl, 2′,3-diCl-biphen-4-yl, 3,5-di(2-chlorophenyl)phenyl, 3,5-di(2-methoxyphenyl)-phenyl, 2′,3-di(2-methylphenyl)-thiophen-5-yl, 2,3-di(2-chlorophenyl)thiophen-5-yl, 3-(2-chlorophenyl)-isoxazol-5-yl, 5′-carboxy-2′-methyl-biphen-4-yl, 5′-carboxy-2′-chloro-biphen-4-yl, 5′-carboxy-2′-fluorobiphen-4-yl, 4′-carboxy-2′-chlorobiphen-4-yl, 2,3-diphenylthiophen-5-yl, 2-(2-chlorophenyl)pyridin-5-yl, 2-(2,6-dichlorophenyl)-pyridin-5-yl, 2-(2-CF₃phenyl)pyridin-5-yl, 2-(5-carboxy-2-chlorophenyl)-pyridin-5-yl, 2-(2,6-dichlorophenyl)-3-chloropyridin-5-yl, 3-chloro-2-(2-trifluoromethylphenyl)pyridin-5-yl, 2-(4-carboxy-2-chlorophenyl)pyridin-5-yl, 1-methyl-3-trifluoromethyl-1H-thieno[2,3-c]pyrazol-5-yl, 1,3-dimethyl-1H-thieno[2,3-c]pyrazol-5-yl, 1-methyl-3-phenyl-1H-thieno[2,3-c]pyrazol-5-yl, 4-(3-methylpyridin-2-yl)-phenyl, 4-(5-carboxy-2-methylthiophen-3-yl)phenyl, 4-(5-carboxy-2-chlorothiophen-3-yl)phenyl, 2,3-diphenylthiophen-5-yl, 2,3-diphenylthiophen-5-yl, 5′-carboxy-2′-chlorobiphen-4-yl, 2-(2-chlorophenyl)pyridin-5-yl, 1-oxo-2-(2-Clphenyl)pyridin-5-yl, 2-(2-Clphenyl)-pyridin-5-yl, 5′-carboxy-2′-chloro-biphen-4-yl, 3,5-dithiophen-3-ylphenyl, 3,5-difuran-2-ylphenyl, 4-(2-chlorophenyl)thiophen-2-yl, 3-chloro-2′-methylbiphen-4-yl, 1-oxo-2-(2,6-dichlorophenyl)pyridin-5-yl, 1-oxo-2-(2-methylphenyl)pyridin-5-yl, 4′-carboxy-2′-methylbiphen-4-yl, 1-oxo-3-chloro-2-(2-chlorophenyl)pyridin-5-yl, 3-chloro-2-(2-trifluoromethylphenyl)pyridin-5-yl, 3-chloro-2-(2-methylphenyl)pyridin-5-yl, 1-oxo-2-(2-chlorophenyl)pyridin-5-yl, 4′-carboxy-2′6′-dichlorobiphen-4-yl, 4′-carboxy-3,2′-dichlorobiphen-4-yl, 4-chloro-2′-methylbiphen-3-yl, 2′-chloro-5′-methoxycarbonylbiphen-4-yl, 2′-chloro-5′-tert-butoxycarbonylbiphen-4-yl, 2′-chloro-4′-methoxycarbonylbiphen-4-yl, 2-(2-chloro-4-methoxycarbonylphenyl)pyridin-5-yl, 2-(2,5-dimethylphenyl)pyridin-5-yl, 2,2′,3′-trichlorobiphen-4-yl, 2,2′-dichlorobiphen-4-yl, 3-(5-methylthiophen-2-yl)phenyl, 2-chloro-3-(2-chlorophenyl)thiophen-5-yl, or 2-chloro-3-phenylthiophen-5-yl, preferably, 2′-chlorobiphen-4-yl, 3,2′-dichlorobiphen-4-yl, 5′-carboxy-2′-chlorobiphen-4-yl, 2′-chloro-5′-methoxycarbonylbiphen-4-yl, 5′-carboxy-2′6′-dichlorobiphen-4-yl, 2-(2,6-dichlorophenyl)pyridin-5-yl, or 2-(2-chlorophenyl)pyridin-5-yl; and

R² is hydrogen, methyl, 2-propyl, 2-methylpropyl, 2-cyclohexylpropyl, 2,6-diF-benzyl, 2,6-diF-4-OCH₃benzyl, 2(S)-phenylpropyl, 2(R)-phenylpropyl, 2-methyl-2-phenylpropyl, 2-phenylethyl, 2-(2-methoxyphenyl)propyl, 2-(trifluoromethoxyphenyl)propyl, 2(R)-phenylprop-2-enyl, benzyl, hydrogen, 1-methylindol-3-yl, 4-methylindol-3-ylmethyl, or thiazol-2-ylmethyl, preferably 2,6-diF-benzyl, 2,6-diF-4-OCH₃benzyl, 2(S)-phenylpropyl, 2(R)-phenylpropyl, or 2-(2-methoxyphenyl)propyl.

II. Another preferred group of compounds of Formula (I) is that wherein:

R¹ is a group of formula: 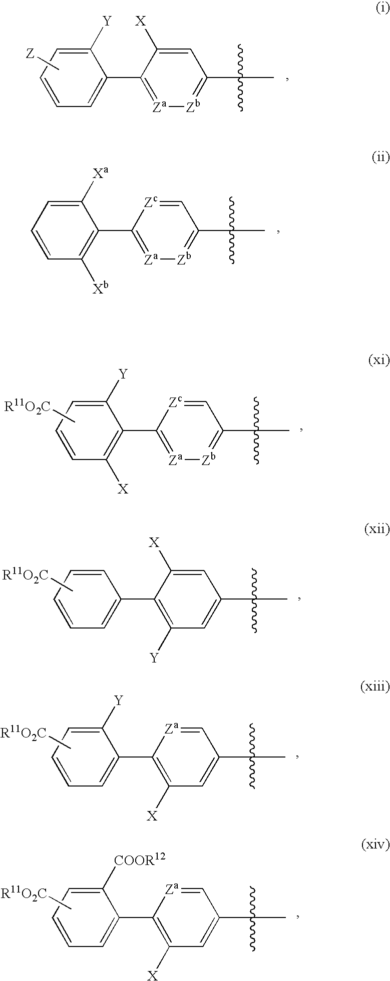
(xviii) 1-methyl-1H-thieno[2,3-c]pyrazol-5-yl where the 3-position of the pyrazole ring is substituted with alkyl or phenyl optionally substituted with alkyl, halo, haloalkyl, haloalkoxy, or alkoxy;
(xxi) biphen-4-yl;
(xxii) 4-alkoxycarbonylbiphen-4-yl;
(xxii) 4-carboxybiphen-4-yl; or
(xxiii) 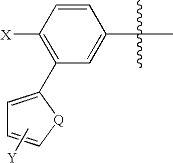
where:

Z^{a} and Z^{b} are independently —CX— or —N— and Z^{c} is selected from —CH— and —N— provided that if an R¹ group contains Z^{a}, Z^{b}, and Z^{c} simultaneously then, when Z^{c} is —N—, then Z^{a} is —N— or —CX— and Z^{b} is —H—; and when Z^{b} is —N— then both Z^{a} and Z^{c} cannot be —N— simultaneously;

Q is —NR— where R is hydrogen or alkyl, —O—, or —S—;

X and Y are independently selected from hydrogen, halo, alkyl, alkoxy, haloalkyl, or haloalkoxy provided that both X and Y are not simultaneously hydrogen;

Z is halo, alkyl, alkoxy, haloalkyl, or haloalkoxy;

X^{a} and X^{b} are independently selected from alkyl, halo, alkoxy, haloalkyl, or haloalkoxy;

R² is selected from the group consisting of hydrogen, cyclopentyl, cyclohexyl, cycloheptyl, methyl, ethyl, n-propyl, 2-propyl, 2-methylpropyl, 2-ethylbutyl, 3-methylbutyl, thiazolylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2,4,4-trimethylpentyl, 1-methylindol-3-ylmethyl, 4-methylindol-3-ylmethyl, 2-napth-1-ylpropyl, benzyloxymethyl, 2-cyclohexylpropyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), benzyl (where the phenyl ring in the benzyl group is optionally substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino), heteroaryl(C₃₋₆)alkyl, and 1-heteroaryl(C₃₋₆)cycloalkylmethyl, and furthermore wherein the alkyl chain in the above groups is optionally substituted with one to six halo;

R³ is hydrogen; or

R² and R³ together with the carbon atom to which they are attached form (C₄₋₈)-cycloalkylene, (C₄₋₈)cycloalkenylene or spirocycloalkylene wherein said (C₄₋₈)cycloalkylene, (C₄₋₈)cycloalkenylene or spirocycloalkylene is optionally substituted with one or two alkyl, alkylidene, or alkenyl;

R⁴ is hydrogen;

R⁵ is hydrogen, alkyl or heteroaryl optionally substituted with alkyl, halo, haloalkyl, haloalkoxy, or alkoxy; or

R⁴ and R⁵ together with the carbon atom to which they are attached form cycloalkylene or heterocycloalkylene; and

each R¹¹ and R¹² are independently alkyl;

or a pharmaceutically acceptable salt thereof.

Within this group of compounds, more preferred groups are where R², R³, R⁴ and R⁵ are as defined in subgroups (A-F) above.

Within the above preferred and more preferred groups, an even more preferred group of compounds is that wherein R² is selected from the group consisting of cyclohexyl, cycloheptyl, thiazol-2-ylmethyl, 2-ethylbutyl, pyrazol-1-ylmethyl, 2-methylpropyl, 2,4,4-trimethylpentyl, 2-napth-1-ylpropyl, 2-phenylprop-2-enyl, 2-phenyl-2-methylpropyl, 2-phenylpropyl, 2-(2-methoxyphenyl)propyl, 4-methylindol-3-ylmethyl, 2-(2,5-dimethylphenyl)propyl, benzyloxymethyl, 2-(2,4-dimethylphenyl)propyl, 2-(2,4-dichlorophenyl)-propyl, 2,6-difluorobenzyl, 2,5-difluorobenzyl, 2,6-difluoro-4-methoxybenzyl, 2-methyl-2-(2-methoxyphenyl)propyl, and 2,3-difluorobenzyl. More preferably, R² is 2,6-difluorobenzyl, 2,6-difluoro-4-methoxybenzyl, 2(S)-phenylpropyl, 2(R)-phenylpropyl, 2-methylpropyl, 2-methyl-2-phenylpropyl, 2-phenylethyl, 2-phenylprop-2-enyl, benzyl, or thiazol-2-ylmethyl. Particularly preferably, R² is 2,6-difluorobenzyl, 2,6-difluoro-4-methoxybenzyl, or 2(S)-phenylpropyl and the stereochemistry at the carbon to which R² is attached is (S) when the Prelog rule places the order of the substituent 1) N, 2) —COOH, 3) R² and 4) H; and (R) when the Prelog rule places the order of the substituent 1) N, 2) R², 3)—COOH and 4) H.

Within these preferred, more preferred groups, a particularly preferred group of compounds is that wherein R¹ is 1,3-dimethyl-1H-thieno[2,3-c]pyrazol-5-yl, 2′-chloro-5′-methoxycarbonylbiphen-4-yl, biphen-4-yl, 4-carboxybiphen-4-yl, 2′-chloro-5′-tert-butoxycarbonylbiphen-4-yl, 2′-chloro-4′-methoxycarbonylbiphen-4-yl, 2,2′,6′-trichlorobiphen-4-yl, 2′-2-dichlorobiphen-4-yl, 2-chloro-3-(2-chlorophenyl)thiophen-5-yl, 2-chloro-3-phenylthiophen-5-yl, 2-(2,5-dimethylphenyl)pyridin-5-yl, 5-(5-methylthiophen-2-yl)phenyl, or 1-methyl-3-phenyl-1H-thieno[2,3-c]pyrazol-5-yl.

A number of different preferences have been given above, and following any one of these preferences results in a compound of this invention that is more presently preferred than a compound in which that particular preference is not followed. However, these preferences are generally independent and additive; and following more than one of these preferences may result in a more presently preferred compound than one in which fewer of the preferences are followed.

### General Synthetic Scheme

Compounds of this invention can be made by the methods depicted in the reaction schemes shown below.

The starting materials and reagents used in preparing these compounds are either available from commercial suppliers such as Aldrich Chemical Co., (Milwaukee, Wis.) or Bachem (Torrance, Calif.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified, if desired, using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of Formula (I) where R¹-R⁵ are as defined in the Summary of the Invention can be prepared as shown in Scheme 1 below.

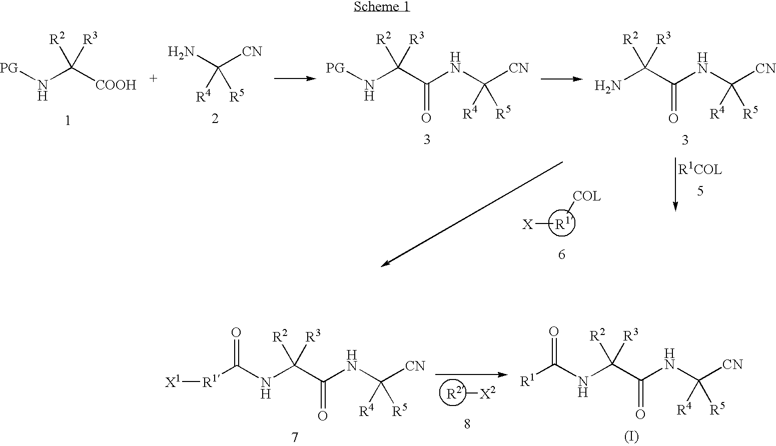

Reaction of an amino acid of formula 1 (where PG is an amino acid protecting group such as tert-butoxycarbonyl, benzyloxycarbonyl, preferably tert-butoxycarbonyl) with an aminomethylnitrile of formula 2 provides an acylaminomethyl nitrile compound of formula 3. The reaction is typically carried out in the presence of a suitable coupling agent e.g., benzotriazole-1-yloxytris-pyrrolidinophosphonium hexafluorophosphate (PyBOP®), O-benzotriazol-1-yl-N,N,N′,N′-tetramethyl-uronium hexafluorophosphate (HBTU), O-(7-azabenzotriazol-1-yl)-1,1,3,3-tetramethyl-uronium hexafluorophosphate (HATU), 1-(3-dimethylaminopropyl)-3-ethylcarbodiimide hydrochloride (EDC), or 1,3-dicyclohexylcarbodiimide (DCC), optionally in the presence of 1-hydroxybenzotriazole (HOBT), and a base such as NN-diisopropylethylamine, triethylamine, N-methylmorpholine, and the like. The reaction is typically carried out at 20 to 30° C., preferably at about 25° C., and requires 2 to 24 h to complete. Suitable reaction solvents are inert organic solvents such as halogenated organic solvents (e.g., methylene chloride, chloroform, and the like), acetonitrile, dimethylformamide, ethereal solvents such as tetrahydrofuran, dioxane, and the like. Preferably, the reaction is carried out with HOBt, EDC in dichloromethane.

Alternatively, this reaction can be carried out by first converting 1 into an active acid derivative such as acid chloride or succinimide ester and then reacting it with 2. The reaction typically requires 2 to 3 h to complete. The reaction conditions utilized in this reaction depend on the nature of the active acid derivative. For example, if it is an acid chloride derivative of 1, the reaction is carried out in the presence of a suitable base (e.g. triethylamine, diisopropylethylamine, pyridine, and the like). Suitable reaction solvents are polar organic solvents such as acetonitrile, N,N-dimethylformamide (DMF), dichloromethane, or any suitable mixtures thereof.

Compounds of formula 1 are either commercially available or they can be prepared by methods well known in the art. For example, 1-N-tert-butoxycarbonylaminocyclohexane carboxylic acid can be prepared from commercially available 1-aminocyclohexane carboxylic acid. 2-Amino-3-(4-methylindol-3-yl)propionic acid can be bought from Bachem. Others can be prepared from 2-benzyloxycarbonylamino-4-(2-methoxyphenyl)pentanoic acid, 2-(2,6-difluorophenyl)alanine, 2-amino-4,6,6-trimethylhetanoic acid, 2-amino-4-methyl-4-phenylpentanoic acid, and 2-amino-4-phenylpent-4-enoic acid whose syntheses are described in working examples below. Compounds of formula 2 such as 2-amino acetonitrile are commercially available. Others can be prepared as described in PCT Applications WO 00/55124, WO 02/20485, the disclosures of which is incorporated herein by reference in their entirety.

Removal of the amino protecting group in 3 provides a compound of formula 4. The reaction conditions employed for removal of the amino protecting group depends on the nature of the protecting group. For example, if the protecting group is tert-butoxycarbonyl, it is removed under acid reaction conditions. Suitable acids are trifluoroacetic acid, hydrochloric acid, methanesulfonic acid, toluenesulfonic acid, and the like. Other suitable reaction conditions for their removal can be found in T. W. Greene, Protecting Groups in Organic Synthesis, John Wiley & Sons, Inc. 1981. The reaction is carried out in an inert organic solvent methylene chloride, tetrahydrofuran, dioxane, dimethylformamide, and the like.

Compound 4 is then converted to a compound of Formula (I) directly by reacting it with a compound of formula 5 where R¹ is as defined in the Summary of the Invention and L is hydroxy or halo under the reaction conditions described above. Alternatively, compound 4 can be converted to a compound of Formula (I) where R¹ is a biaryl group, in two steps by first reacting it with a compound of formula 6 where R¹ is a precursor group to R¹ (i.e., the phenyl, pyridinyl, or pyrimidinyl ring in R¹ that is attached to the carbonyl carbon of the amido group), X is halo, preferably bromo, triflate, mesylate, and the like, and L is as defined above, under the reaction conditions described above to provide a compound of formula 7. Compound 7 is then reacted with a compound of formula 8 where R²′ is a second ring in R¹ group and X² is a boronic acid or boronic ester under Suzuki coupling reaction conditions to provide a compound of Formula (I). Conversely, X¹ is the boronic acid or ester and X² is halide, mesylate, tosylate, and the like.

Acid derivatives of the formula 5 where L is a halogen can be prepared by reacting the corresponding acids with a halogenating agent such as oxalyl chloride, thionyl chloride, and the like. Acids of formula R¹COOH are either commercially available or they can be prepared from commercially available starting materials by methods known in the art. For example, 1-(4-aminosulfonylphenyl)-5-(4-chlorophenyl)pyrazol-3-ylcarboxylic acid and 1-methyl-3-trifluoromethyl-1H-thieno[2,3-c]pyrazol-5-ylcarboxylic acid are commercially available from Bionet. Other compounds of formula 5 can be prepared as described in working examples below. Compounds of formula 6 and 8 are either commercially available or they can be prepared by method well known in the art.

A compound of Formula (I) can be converted to other compounds of Formula (I). For example, a compound of Formula 1 where R⁴ and R⁵ together form tetrahydrothiopyran can be converted to a corresponding compound of Formula (I) where R⁴ and R⁵ together form 1,1-dioxotetrahydrothiopyran by under oxidation reaction conditions. Suitable oxidizing agent are OXONE or m-chloro-perbenzoic acid, and the like. Similarly, compounds that contain a pyridyl ring in R¹ can be oxidized to pyridine N-oxide using m-chloro perbenzoic acid.

Alternatively, compounds of Formula (I) where R¹-R⁵ are as defined in the Summary of the Invention can be prepared as shown in Scheme 2 below.

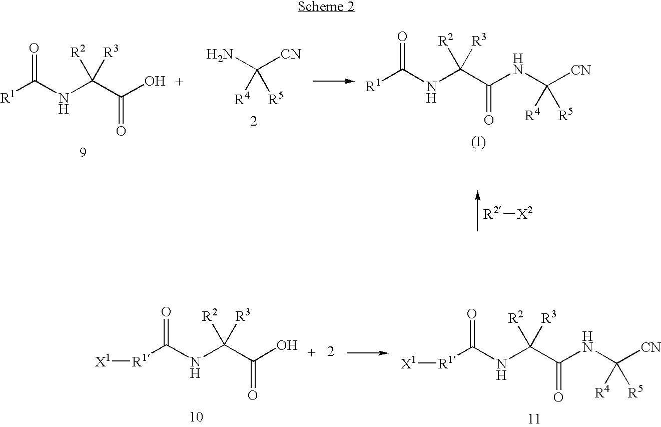

Compounds of Formula (I) can alternatively be prepared by reacting a compound of formula 9 or an acid derivative thereof such as acid halide with a compound of formula 2 under reaction conditions described above.

Compounds of formula 9 can be prepared by methods well known in the art. For example, a compound of formula 9 can be prepared by reacting an aminoacid of formula R²R³C(NH₂)COOH with an acid derivative of the formula R¹COL or X¹—R¹′—COL where L is a suitable leaving group such as chloro and the like and X¹ and R¹′ are as defined in Scheme 1 above. Specifically, a compound of formula 9 where R² is 2,6-difluorobenzyl, R³ is hydrogen, and R¹ is 2′-chlorobiphen-4-yl can be prepared by reacting 2,6-difluorophenylalanine with 2′-chlorobiphen-4-ylcarbonyl chloride in the presence of base such as triethylamine and in a suitable organic solvent such as acetonitrile or aqueous inorganic base such as sodium hydroxide in dioxane. Amino acids of the formula R²R³C(NH₂)COOH are either commercially available or they can be prepared by methods known in the art. Syntheses of some such amino acids are described in working examples below.

Acid derivatives of the formula R¹COL where L is a halogen can be prepared by reacting the corresponding acids with a halogenating agent such as oxalyl chloride, thionyl chloride, and the like. Acids of formula R¹COOH are either commercially available or they can be prepared from commercially available starting materials by methods known in the art. For example, 1-(4-aminosulfonylphenyl)-5-(4-chlorophenyl)pyrazol-3-ylcarboxylic acid and 1-methyl-3-trifluoromethyl-1H-thieno[2,3-c]pyrazol-5-ylcarboxylic acid are commercially available from Bionet. Syntheses of some compounds of formula 9 such as 2S-(2′-chlorobiphen-4-ylcarbonylamino)-4-ethylhexanoic acid, 2-[(2′-chlorobiphen-4-carbonyl)amino]-4-phenylpentanoic acid, 2-(2′-chlorobiphen-4-carbonylamino)-3-thiazol-2-ylpropionic acid methyl ester, (2S)-(2′-chlorobiphen-4-ylcarbonylamino)-3-(2,6-difluorophenyl)propionic acid is described in detail below.

Alternatively, a compound of Formula (I) can be prepared from a compound of formula 10 where X¹ and R¹′ are as defined in Scheme 1 above by reacting 10 with an aminoacetonitrile 2 to provide a compound of formula 11 which is then converted to a compound of Formula (I) using Suzuki coupling conditions described in Scheme 1 above.

### Pharmacology and Utility

The compounds of the invention are cysteine protease inhibitors. In particular the compounds of the invention inhibit the activity of cathepsins B, L, K, F and/or S and, as such, are useful for treating diseases in which cathepsin B, L, K, F and/or S activity contributes to the pathology and/or symptomatology of the disease. For example, the compounds of the invention are useful in treating tumor invasion and metastasis, in particular as anti-angiogenic agents, rheumatoid arthritis, osteoarthritis, pneumocystis carinii, acute pancreatitis, inflammatory airway disease, atherosclerosis, restenosis, and bone and joint disorders. Furthermore, the compounds of the invention are useful in treating bone resorption disorders, e.g., osteoporosis, endometrosis and atheroclerosis. The compounds of the invention also are useful in treating autoimmune disorders, including, but not limited to juvenile onset diabetes, multiple sclerosis, pemphigus vulgaris, Graves disease, myasthenia gravis, systemic lupus erythemotasus, rheumatoid arthritis and Hashimoto's thyroiditis. The compounds of the invention also are useful in treating allergic disorders, including, but not limited to asthma; and allogeneic immune reponses, including, but not limited to, organ transplants or tissue grafts.

The cysteine protease inhibitory activities of the compounds of the invention can be determined by methods known to those of ordinary skill in the art. Suitable in vitro assays for measuring protease activity and the inhibition thereof by test compounds are known. Typically, the assay measures protease-induced hydrolysis of a peptide-based substrate. Details of assays for measuring protease inhibitory activity are set forth in Biological Examples 1-5, infra.

### Administration and Pharmaceutical Compositions

In general, compounds of Formula (I) will be administered in therapeutically effective amounts via any of the usual and acceptable modes known in the art, either singly or in combination with another therapeutic agent. A therapeutically effective amount may vary widely depending on the severity of the disease, the age and relative health of the subject, the potency of the compound used and other factors. For example, therapeutically effective amounts of a compound of Formula (I) may range from about 10 micrograms per kilogram body weight (μg/kg) per day to about 20 milligram per kilogram body weight (mg/kg) per day, typically from about 100 μg/kg/day to about 10 mg/kg/day. Therefore, a therapeutically effective amount for a 80 kg human patient may range from about 1 mg/day to about 1.6 g/day, typically from about 1 μg/day to about 100 mg/day. In general, one of ordinary skill in the art, acting in reliance upon personal knowledge and the disclosure of this Application, will be able to ascertain a therapeutically effective amount of a compound of Formula (I) for treating a given disease.

The compounds of Formula (I) can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of Formula (I) in combination with at least one pharmaceutically acceptable excipient. Acceptable excipients are non-toxic, aid administration, and do not adversely affect the therapeutic benefit of the active ingredient. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from water, ethanol, glycerol, propylene glycol and various oils, including those of petroleum, animal, vegetable or synthetic origin (e.g., peanut oil, soybean oil, mineral oil, sesame oil, or the like). Preferred liquid carriers, particularly for injectable solutions, include water, saline, aqueous dextrose and glycols.

The amount of a compound of Formula (I) in the composition may vary widely depending upon the type of formulation, size of a unit dosage, kind of excipients and other factors known to those of skill in the art of pharmaceutical sciences. In general, a composition of a compound of Formula (I) for treating a given disease will comprise from 0.01% w to 10% w, preferably 0.3% w to 1% w, of active ingredient with the remainder being the excipient or excipients. Preferably the pharmaceutical compositions are administered in a single unit dosage form for continuous treatment or in a single unit dosage form ad libitum when relief of symptoms is specifically required. Representative pharmaceutical formulations containing a compound of Formula (I) are described below.

### EXAMPLES

The following preparations and examples are given to enable those skilled in the art to more clearly understand and to practice the present invention. They should not be considered as limiting the scope of the invention, but merely as being illustrative and representative thereof.

### Synthetic Examples

### General Procedures

### Example A

### Synthesis of 2(RS)-benzyloxycarbonylamino-4(RS)-(2-methoxyphenyl)pentanoic acid

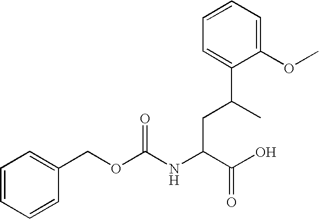

To d,l-2-methoxy-α-methylbenzyl alcohol (0.5 g, 3.29 mmol) was added 48% aq. HBr (2 mL) and the reaction mixture was stirred rapidly for 1.5 h. The reaction mixture was diluted with hexane (30 mL), washed with water, dried with MgSO₄ and evaporated under vacuum. The crude d,l-2-methoxy-α-methylbenzyl bromide was added to a solution of tributyltin hydride (0.67 mL, 2.49 mmol), Z-dehydroalanine methyl ester (0.25 g, 1.06 mmol), and 2,2′-azobisisobutyronitrile (15 mg, 0.09 mmol) in benzene (5 mL). The reaction mixture was heated at 80° C. under a nitrogen atmosphere for 5 h. Benzene was removed under vacuum and the residue was dissolved in methanol (20 mL). 2N KOH (5 mL) was added and the mixture was rapidly stirred at room temperature over night. Methanol was removed under vacuum and the residue was diluted with water (20 mL). The aqueous solution was washed with ether to remove the tin byproducts. The aqueous layer was acidified with 6N aq. HCl and the product was extracted with ethyl acetate. The combined organic layers were washed with brine, dried with MgSO₄ and evaporated under vacuum to give 2-benzyloxy-carbonylamino-4-(2-methoxyphenyl)pentanoic acid (190 mg, 0.53 mmol) as a mixture of diastereomers in sufficiently pure form to be used without further purification. (MS: (M⁺+H) 358, (M⁺−H) 356)

Following the procedure described above, and utilizing appropriate starting materials the following amino acids were prepared:
2-benzyloxy-carbonylamino-4-(2-methoxyphenyl)hexanoic acid;
2-benzyloxy-carbonylamino-4-(4-fluorophenyl)pentanoic acid;
2-benzyloxy-carbonylamino-4-(4-chlorophenyl)pentanoic acid;
2-benzyloxy-carbonylamino-4-(4-methoxyphenyl)pentanoic acid;
2-benzyloxy-carbonylamino-4-(2-trifluoromethylphenyl)pentanoic acid;
2-benzyloxy-carbonylamino-4-(3-trifluoromethylphenyl)pentanoic acid;
2-benzyloxy-carbonylamino-4-(napth-1-yl)pentanoic acid;
2-benzyloxy-carbonylamino-4-(2,6-dimethylphenyl)pentanoic acid;
2-benzyloxy-carbonylamino-4-(2,4-difluorophenyl)pentanoic acid;
2-benzyloxy-carbonylamino-4-(2,4-dimethylphenyl)pentanoic acid;
2-benzyloxy-carbonylamino-4-(2,5-dimethylphenyl)pentanoic acid; and
2-benzyloxy-carbonylamino-4-(2,4-dichlorophenyl)pentanoic acid.

The benzyloxycarbonyl group can be removed as described in Example B below to give the corresponding free amino acid.

### Example B

### Synthesis of 2(S)-2,6-difluorophenylalanine

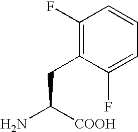 Step 1

N-(Benzyloxycarbonyl)-α-phosphonoglycine trimethyl ester (Aldrich #37,635-3; 6.7 g, 20 mmol) and 1,8-diazabicyclo[5,4,0]undec-7-ene (Aldrich #13, 900-9; 3.3 mL, 22 mmol) were dissolved in methylene chloride (11 mL) and stirred at room temperature for 15 min., and then cooled to <−30° C. A solution of 2,6-difluorobenzaldehyde (1.9 mL, 20 mmol) in methylene chloride (25 mL) was added to the reaction mixture dropwise over 20 minutes. The reaction mixture was stirred for another 20 min., and then allowed to warm up to room temperature for 30 min. The reaction mixture was then poured into ethyl ether (300 mL) and washed with 1N HCl, brine and dried over MgSO₄. Rotary evaporation gave 2-benzyloxycarbonylamino-3-(2,6-difluorophenyl)acrylic acid methyl ester. This crude product was purified by chromatography on a Medium Pressure Liquid Column (MPLC) eluting with 20% ethyl acetate/80% hexane to give pure product (5 g, 72% yield, liquid).

Step 2

A mixture of 2-benzyloxycarbonylamino-3-(2,6-difluorophenyl)acrylic acid methyl ester (14.4 mmol), and catalyst, (+)-1,2-bis-[(2S,5S)2,5-diethylphopholano]benzene (cyclooctadiene)rhodium (1) trifluoromethanesulfonate (Strem. Chemical #45-0151; 104 mg, 0.14 mmol) was dissolved in ethanol (150 mL). Hydrogenation was performed at 50 psi H₂ at room temperature over 2 days. The solvent was then removed by rotary evaporation to give (2S)-benzyloxycarbonylamino-3-(2,6-difluorophenyl)propionic acid methyl ester.

Step 3

(2S)-Benzyloxycarbonylamino-3-(2,6-difluorophenyl)propionic acid methyl ester (5 g, 14.4 mmol) was dissolved in methanol (60 mL) and cooled on ice. 1N NaOH (22 mL, 22 mmol) was added dropwise over 15 minutes. The reaction mixture was removed from cooling and continue stirring at room temperature for 4 h. The solvent was then removed by rotary evaporation. The residue was treated with water (100 mL) and then with 1N HCl to adjust the pH to 4. The product was extracted with ethyl acetate (300 mL, 200 mL). Evaporation of the solvent and crystallization of the residue from methylene chloride/hexane gave (2S)-benzyloxycarbonylamino-3-(2,6-difluorophenyl)propionic acid (4.6 g, 13.7 mmol, 94% yield).

Step 4

(2S)-Benzyloxycarbonylamino-3-(2,6-difluorophenyl)-propionic acid was hydrogenated at 50 psi in ethanol (25 mL) in the presence of 5% palladium on activated carbon (600 mg) for 24 h. The catalyst was removed by filtration through celite and the solvent evaporated to give a residue which was crystalized from ethyl ether to give 2(S)-2,6-difluorophenylalanine (2.2 g, 11 mmol, 80% yield).

H¹ NMR(DMSO-d): 7.28(1H, m), 7.0(2H, t, 7.6 Hz), 2.77(2H, m). MS: 202.2(M+1), 199.7(M−1).

### Example C

### Synthesis of 2(RS)-amino-4-(RS)-6,6-trimethylheptanoic acid

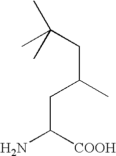 Step 1

To a mixture of the 3,5,5-trimethylhexanal (17.4 mL, 0.10 mol), ammonium chloride (53.5 g, 0.205 mol) and diethyl ether (113 mL) was added sodium cyanide (7.35 g, 0.15 mol) in water (38 mL). The reaction mixture was allowed to stir vigorously for 16 h. The layers were separated. The aqueous layer was extracted with diethyl ether. The combined organic layer was then extracted with 1N HCl. Saturated sodium bicarbonate was then added until 1-cyano-3,5,5-trimethylhexylamine was completely precipitated. Vacuum filtration and washing with 5 mL ice cold water followed by lyophilization gave 1-cyano-3,5,5-trimethylhexylamine (5.805 g, 0.034 mol, 34.5%) as a white solid.

Step 2

1-Cyano-3,5,5-trimethylhexylamine (1.02 g, 5.0 mmol) was treated with 6N HCl (10 mL) and heated at reflux for 30 h. The reaction mixture was allowed to cool to room temperature. Water (50 mL) was added, and the mixture was washed with diethyl ether. The aqueous layer was basified to pH 8.5 with 2N KOH. A white precipitate formed which was collected by vacuum filtration and lyophilized to give 2(RS)-amino-4(RS),6,6-trimethyl-heptanoic acid (364 mg).

### Example D

### Synthesis of 2(RS)-amino-4-methyl-4-phenylpentanoic acid

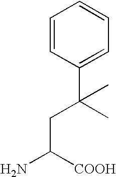 Step 1

4-Methyl-4-phenyl-1-pentene was prepared by reacting 2-phenyl-2-propanol with 3-(trimethylsilyl)propene by the method of Cella, J. Org. Chem., 1982, 47, 2125-2130.

Step 2

4-Methyl-4-phenyl-1-pentene was ozonolyzed at −78° C. in dichloromethane followed by dimethyl sulfide quenching to give crude product which was purified by silica gel chromatography to give 3-methyl-3-phenylbutanal which was then converted to the title compound by proceeding as described in Example C above.

### Example E

### Synthesis of 2(S)-amino-4-phenylpent-4-enoic acid

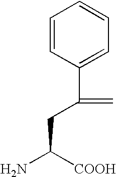 Step 1

Methyl triphenylphosphonium bromide (1.12 g, 3.14 mmol, 2.0 equiv.) was dissolved in THF (15 mL) and cooled to 0° C. Sodium bis(trimethylsilyl)amide (3.14 mL) was added and the reaction mixture was stirred for 30 minutes. 2S-Benzyloxycarbonyl-amino-3-benzoylpropionic acid ethyl ester (0.54 g, 1.57 mmol, 1.0 equiv. prepared by procedures outlined in Synthesis 2001, No. 7, p. 1007) was dissolved in THF (5 mL) and added to the reaction. After warming to room temp., the reaction mixture was quenched with saturated ammonium chloride and partitioned between water and EtOAc. After concentration of the organic phase, purification was carried out with flash chromatography to provide 2-benzyloxycarbonylamino-4-phenyl-pent-4-enoic acid ethyl ester. Removal of the benzyloxycarbonyl group as described above, provided the title compound.

### Example F

### Synthesis of 2(RS)-benzyloxycarbonylamino-4-ethylhexanoic acid

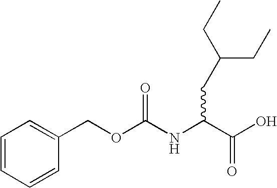 Step 1

A mixture of 2-benzyloxycarbonylaminomalonic acid diethyl ester (1.237 g, prepared as described in C. M. Bladon, J. Chem. Soc. Perkin Trans. I, 1990, 1151-1158), iodo-2-ethylbutane (1.272 g) and lithium hydroxide (0.287 g) in N-methylpyrrolidone (8 mL) was stirred for 2 days at room temperature and then diluted with ice water. The aqueous solution was extracted with ether and the product purified by chromatography on silica gel to give 2-benzyloxycarbonylamino-2-(2-ethylbutyl)malonic acid diethyl ester (0.520 g).

Step 2

A solution of 2-benzyloxycarbonylamino-2-(2-ethylbutyl)malonic acid diethyl ester (0.520 g) in ethanol (5 mL) was treated with sodium hydroxide (2.91 mL, 1 N) and then stirred at room temperature for 8 h. The reaction mixture was diluted with water and acidified with HCl and the product was then extracted with ethyl acetate to give 2-benzyloxycarbonylamino-2-(2-ethylbutyl)malonic acid monoethyl ester (0.461 g).

Step 3

2-Benzyloxycarbonylamino-2-(2-ethylbutyl)malonic acid monoethyl ester was heated at 75° C. in ethanol (5 mL) with sodium hydroxide (5 mL, 1 N) for 3 h and 2-benzyloxycarbonylamino-2-(2-ethylbutyl)malonic acid was isolated by extraction of the acidified reaction mixture. 2-Benzyloxycarbonylamino-2-(2-ethylbutyl)malonic acid was heated at 103° C. for 1 h and the resulting residue was purified by column chromatography on silica gel to give 2RS-benzyloxycarbonylamino-4-ethylhexanoic acid (0.220 g).

### Example G

### Synthesis of 2′-chlorobiphenyl-4-carboxylic acid

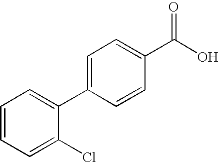 Step 1

4-Bromobenzoic acid ethyl ester (3.91 g, 17.0 mmol, 1.0 equiv.) was combined with Pd tetrakis(triphenylphosphine) (0.98 g, 0.85 mmol, 0.05 equiv.), ethanol (19 mL), and toluene (98 mL). The reaction mixture was stirred for 20 min at room temperature. To this was added potassium carbonate (11.74 g, 85.0 mmol, 5.0 equiv.) and 2-chlorophenylboronic acid (4.0 g, 25.6 mmol, 1.5 equiv). The suspension was heated to 70° C. for 6 h. The reaction mixture was diluted with ether (400 mL) and extracted with water (400 mL). The organic layer was washed with brine and dried over anhydrous magnesium sulfate. After filtering and concentration the resulting oil was purified by flash chromatography (7% EtOAc/hexanes as eluent to afford 3.16 g of 2′-chlorobiphenyl-4-carboxylic acid ethyl ester.

Step 2

2′-Chlorobiphenyl-4-carboxylic acid ethyl ester was dissolved in MeOH (141 mL). To this was added sodium hydroxide (2.35 g) in water (30 mL). The solution was stirred for 6 h at room temperature, then diluted with 250 mL of water, followed by exatraction with ether (200 mL). The aqueous layer was acidified with conc. hydrochloric acid, extracted with ethyl acetate (300 mL), dried then concentrated to give 2′-chloro-biphenyl-4-carboxylic acid (2.81) as a white solid.

Following the procedure described in Example G above, the following starting materials were prepared:
2′-methylbiphenyl-3-carboxylic acid;
2′-methoxybiphenyl-3-carboxylic acid;
4-chlorobiphenyl-3-carboxylic acid;
4-chloro-2′-methyl-biphenyl-3-carboxylic acid;
4-(2-methylphenyl)thiophen-2-ylcarboxylic acid;
4-(2-methoxyphenyl)thiophen-2-ylcarboxylic acid;
4-(2-chlorophenyl)thiophen-2-ylcarboxylic acid;
2-(2-methylphenyl)-3-methoxythiophen-4-ylcarboxylic acid;
2-(2-methoxyphenyl)-3-methoxythiophen-4-ylcarboxylic acid;
2-(2-methylphenyl)thiophen-5-ylcarboxylic acid;
2-(2-methoxyphenyl)thiophen-5-ylcarboxylic acid;
2-(2-methylphenyl)furan-5-ylcarboxylic acid;
2-(2-methoxyphenyl)furan-5-ylcarboxylic acid;
2-(2,6-dichlorophenyl)thiophen-5-ylcarboxylic acid;
3,5-diphenylbenzoic acid;
3,5-di(2-methoxyphenyl)benzoic acid;
3,5-di(3-methoxyphenyl)benzoic acid;
3,5-dithiophen-3-ylbenzoic acid;
3,5-dipyridin-4-ylbenzoic acid;
3,5-difuran-2-ylbenzoic acid;
3,5-di(2-chlorophenyl)benzoic acid;
2,3-diphenylthiophen-5-carboxylic acid;
2,3-di(2-methoxyphenyl)thiophen-5-carboxylic acid;
2,3-di(2-methylphenyl)thiophen-5-carboxylic acid;
2,3-difuran-2-ylthiophen-5-carboxylic acid;
2,3-di(2-chlorophenyl)thiophen-5-carboxylic acid; and
4,5-diphenylthiazol-2-ylcarboxylic acid;

Starting materials for preparing the above acid via the Suzuki coupling were either commercially available from Aldrich, Frontier, or Lancaster or they were prepared from the synthetic procedure described in Heterocycles, 1995, 41, 1659-1666; Bioorg. Med. Chem., 1999, 7, 1559-1566;

### Example H

### Synthesis of 3,2′-dichlorobiphenyl-4-carboxylic acid

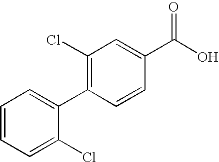 Step 1

3-Chloro-4-hydroxybenzoic acid methyl ester (3.0 g, 16.5 mmol, 1.0 equiv.) was dissolved in dichloromethane (60 mL) and cooled in an ice-water bath. After addition of 2,6-lutidine (9.6 mL), triflic anhydride (4.0 mL) was added dropwise. The reaction mixture was warmed to room temperature and subsequently stirred an additional 16 h. The reaction mixture was diluted with water and ethyl acetate. The organic layer was washed with 1 N HCl, saturated sodium bicarbonate, and dried over anhydrous sodium sulfate and concentrated to give 3-chloro-4-trifluoromethane-sulfonyloxybenzoic acid which was reacted with 2-chlorophenylboronic acid to give 3,2′dichlorobiphenyl-4-carboxylic acid methyl ester which was converted to the free acid as described above.

Utilizing the procedure described in Example H above, but substituting 3-chloro-4-hydroxybenzoic acid methyl ester with 6-hydroxynicotinic acid provided 2-(2-chlorophenyl)-pyridine-5-carboxylic acid.

### Example I

### Synthesis of 2(S)-(2′-chlorobiphen-4-ylcarbonylamino)-4-phenylpent-4-enoic acid

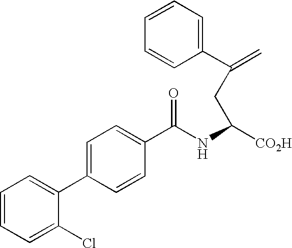 Step 1

α-Methylstyrene was heated with N-bromosuccinamide in carbon tetrachloride to 140° C. until foaming stopped. The reaction mixture was cooled to room temperature and filtered. α-Bromomethylstyrene and β-bromo-α-methylstyrene were obtained by distillation in an 80:20 ratio and used as such in the next step.

Step 2

Sodium ethoxide was generated from sodium metal in ethanol. To this solution was added diethyl malonate. After stirring the reaction mixture for 5 minutes a mixture of α-bromomethylstyrene and β-bromo-α-methylstyrene was added and the reaction mixture was heated at 50° C. for 1 h and then allowed to stir at room temperature for 16 h. The reaction mixture was poured into ice water and extracted with ether, dried and concentrated. The crude product was purified from the mixture by silica gel chromatography to give 2-(2-phenylallyl)malonic acid dimethyl ester.

Step 3

2-(2-Phenylallyl)malonic acid dimethyl ester was heated with potassium hydroxide in water and ethanol mixture at 95° C. over 2 h. Ethanol was removed and the basic layer was washed with diethyl ether, acidified and extracted with ethyl acetate, dried and concentrated to give crude 2-(2-phenylallyl)malonic acid which upon heating at 145° C. gave 4-phenylpent-4-enoic acid, which was purified by silica gel chromatography.

### Step 4

4-Phenylpent-4-enoic acid was converted to 4-phenylpent-4-enoyl chloride as described in Example K, step 4 below. 4-Phenylpent-4-enoyl chloride was then converted to 2(S)-(2′-chlorobiphen-4-ylcarbonylamino)-4-phenylpent-4-enoic acid by proceeding as described in Example M, Steps 2-6 described below.

### Example J

### Synthesis of 2(S)-benzyloxycarbonylamino-3-pyrazol-1-ylpropionic acid

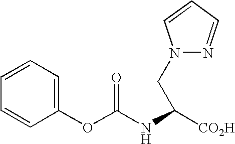

The title compound was prepared by treating S-benzyloxycarbonylserine-β-lactone with pyrazole in acetonitrile at 60° C. for 16 h (see J. Am. Chem. Soc., 1985, 107, 7105-7109).

Following the procedure described above, but substituting pyrazole with 1,2,4-triazole and 1,2,3-triazole provided 2(S)-benzyloxycarbonylamino-3-[1,2,4]-triazol-1-ylpropionic acid and 2(S)-benzyloxycarbonylamino-3-[1,2,3]-triazol-1-ylpropionic acid respectively.

### Example K

### Synthesis of 2(S)-(2′-chlorobiphen-4-ylcarbonylamino)-3-(2,6-difluorophenyl)propionic acid

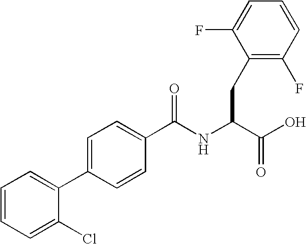 Step 1

A solution of (5S,6R)-4-(tert-butyoxycarbonyl)-5,6-diphenyl-2,3,5,6-tetrahydro-4H-1,4-oxazine-2-one (10.59 g, 0.03 mol) and 2,6-difluorobenzyl bromide (7.038 g, 0.034 mol) in tetrahydrofuran (150 mL) was cooled to −60° C. and then treated with sodium hexamethyldisilazane (32 mL of 1N in tetrahydrofuran) by slow addition over 20 minutes. The reaction mixture was stirred at −67° C. for 105 minutes and then poured into cold water. The product was extracted with ethyl acetate. The extracts were dried and concentrated to 120 mL and cooled to 0° C. Filtration in two crops gave (3S,5S,6R)-4-(tert-butyloxycarbonyl)-3-(2,6-difluorophenylmethyl)-5,6-diphenyl-2,3,5,6-tetrahydro-4H-1,4-oxazine-2-one (8.90 g).

Step 2

A solution of (3S,5S,6R)-4-(tert-butyloxycarbonyl)-3-(2,6-difluorophenylmethyl)-5,6-diphenyl-2,3,5,6-tetrahydro-4H-1,4-oxazine-2-one (7.28 g, 15.2 mmol) in methylene chloride (150 mL) was cooled to 0° C. and treated with trifluoroacetic acid (15 mL) and then stirred at room temperature for 4.5 h. The reaction mixture was cooled to 0° C. and treated with triethylamine (27.8 mL). The reaction mixture was then concentrated at reduced pressure, diluted with cold water and the product extracted with ethyl acetate to give (3S,5S,6R)-3-(2,6-difluorophenylmethyl)-5,6-diphenyl-2,3,5,6-tetrahydro-4H-1,4-oxazine-2-one (5.86 g) as an oil which was used in the next step without purification.

Step 3

A solution of (3S,5S,6R)-3-(2,6-difluorophenylmethyl)-5,6-diphenyl-2,3,5,6-tetrahydro-4H-1,4-oxazine-2-one (5.86 g, 0.152 mol) in tetrahydrofuran (25 mL) and methanol (25 mL) was hydrogenated in the presence of palladium chloride (0.317 g) at 55 psi for 16 h. More (0.100 g) palladium chloride was added to the reaction mixture and the hydrogenation continued for an additional 3 h. The catalyst was removed by filtration, the solvents were removed at reduced pressure and the residue was acidified with 1N aqueous hydrochloric acid. After washing with ethyl acetate the aqueous layer was neutralized to pH 6.9 at 0° C. with 1N sodium hydroxide and then evaporated. The resulting solid was slurried and filtered with methanol (50 mL) twice. Cooling of the methanolic extracts on ice then gave 2(S)-(2,6-difluorophenyl)alanine (1.189 g) as white needles.

Step 4

2′-Chloro-4-biphenylcarboxylic acid (2.77 g, 11.9 mmole) was suspended in ethyl acetate (36 mL). A single drop of N,N-dimethylformamide was added and the suspension cooled in an ice bath. Oxalyl chloride was added dropwise over a 5 minute period, the bath removed and the resulting solution stirred for an additional 20 minutes. The solvent removed in vacuo and the resulting 2′-chloro-4-biphenylcarbonyl chloride was used immediately without purification.

Step 5

2(S)-2,6-Difluorophenylalanine (2.4 g, 11.9 mmole) was dissolved in 2 N NaOH (11. 9 mL) and dioxane (10 mL) and the solution was cooled in an ice/water bath. A solution of 2′-chloro-4-biphenylcarbonyl chloride in tetrahydrofuran (12 mL) was added concurrently with 2N NaOH solution (5.9 mL) over 20 minutes. The ice bath was removed and the reaction mixture was stirred an additional 45 minutes after which it was acidified to pH 4 with concentrated HCl. The product was extracted with dichloromethane and the ethereal layer was concentrated to give 2(S)-(2′-chlorobiphen-4-ylcarbonylamino)-3-(2,6-difluorophenyl)propionic acid (3.1 g).

Proceeding as described above, but substituting 2′-chloro-4-biphenylcarboxylic acid in Step 4 above with 2-(2-chlorophenyl)-pyridine-5-carboxylic acid provided 2(S)-[2-(2-chlorophenyl)-pyridin-5-ylcarbonylamino]-3-(2,6-difluorophenyl)propionic acid.

### Example L

### Synthesis of 2(R)-amino-4(S)-phenylpentanoic acid and 2(S)-amino-4(S)-phenylpentanoic acid

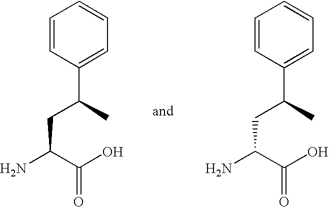 Step 1

### 2(S)-Phenylpropanol was converted to 1-trifluoromethanesulfonyloxy-2(S)-phenylpropane by the procedure given in Org Syn coll. Vol. VIII, p 126.

Step 2

6-Oxo-(2R,3S)-diphenylmorpholine-4-carboxylic acid benzyl ester was converted to 6-oxo-(2R,3S)-diphenyl-5-(2S-phenylpropyl)morpholine-4-carboxylic acid benzyl ester by reacting it with 1-trifluoromethanesulfonyloxy-2(S)-phenylpropane and was then converted to a mixture of 2(R)-amino-4(S)-phenylpentanoic acid and 2(S)-amino-4(S)-phenylpentanoic acid by the methods of Williams, et al., “Methods in Molecular Medicine”, Vol. 23: Peptidomimetics Protocols. Edited by W. M. Kazmierski, Humana Press Inc., Totowa, N.J. p 339-356 and J. Am. Chem Soc., 1991, 113, 9276-9286 respectively.

2(S)-Amino-4(S)-phenylpentanoic acid can also be prepared as a single (S,S) diastereomer from 6-Oxo-(2R,3S)-diphenylmorpholine-4-carboxylic acid benzyl ester as described above by adding all reagents slowly enough to maintain an internal reaction temperature of less than −65° C.

### Example M

### Synthesis of 2(S)-(2′-chlorobiphen-4-ylcarbonylamino)-4-ethylhexanoic acid

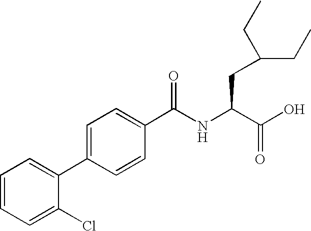 Step 1

A solution of 4-ethylhexanoic acid (11.63 g) (prepared by the method described in P. Daud, C. Kaufman, P. Kaufman, Y. Paik, Tet. Lett., 1985, 26,2279-2282) in ethyl acetate (150 mL) and dimethyl formamide (2 drops) was treated with oxalyl chloride (10.5 mL) at 0° C. and then stirred at room temperature for 50 minutes. The solvents were evaporated to give 4-ethylhexanoyl chloride (11.75 g).

Step 2

A solution of 4(S)-benzyl-2-oxazolidone (5.316 g) in THF (60 mL) was cooled to −65° C. and treated with n-butyllithium (20 mL, 1.6 M) over 20 minutes. A solution of 4-ethylhexanoyl chloride (20.04 g) in THF (5 mL) was added at −65° C. over 20 minutes. After 30 minutes, the reaction mixture was quenched in ice water and the product extracted with ethyl acetate to give 3-(4-ethylhexanoyl)-4(S)-benzyl-2-oxazolidone (8.78 g).

Step 3

3-(4-Ethylhexanoyl)-4(S)-benzyl-2-oxazolidone was converted to 3-(2S-azido-4-ethylhexanoyl)-4(S)-benzyl-2-oxazolidone using potassium hexamethyldisilazide and trisyl azide as described by D. A. Evans, T. C. Britton, J. A. Ellman, R. L. Dorow, J. Am. Chem. Soc., 1990, 112, 4011-4030.

Step 4

3-(2S-Azido-4-ethylhexanoyl)-4(S)-benzyl-2-oxazolidone (0.20 g) in methanol (6 mL) was treated with 5% Pd/C (70 mg) and the hydrogenated at 1 atm. When the reaction was complete the reaction mixture was filtered and the methanol evaporated to give 3-(2S-amino-4-ethylhexanoyl)-4(S)-benzyl-2-oxazolidone.

Step 5

3-(2S-Amino-4-ethylhexanoyl)-4(S)-benzyl-2-oxazolidone was dissolved in acetonitile and treated with HBTU (285 mg), 2′-chloro-4-biphenyl carboxylic acid (175 mg) and N-methylmorpholine (0.22 mL). After stirring at room temperature for 24 h the reaction mixture was diluted with water and the product extracted with ethyl acetate and purified by chromatography on silica gel to give 3-[2S-(2′-chlorobiphen-4-ylcarbonylamino)-4-ethylhexanoyl)]-4(S)-benzyl-2-oxazolidone (0.128 g).

Step 6

3-[2S-(2′-Chlorobiphen-4-ylcarbonylamino)-4-ethylhexanoyl)]-4(S)-benzyl-2-oxazolidone (0.100 g) in THF (5 mL) was cooled on ice and treated with water (1.25 mL), hydrogen peroxide (30% 1.95 mL) and lithium hydroxide (0.0010 g). The reaction mixture was stirred at room temperature for 90 minutes. The reaction mixture was quenched with aqueous sodium sulfite and the product isolated from the acidified aqueous layer to give 2(S)-(2′-chlorobiphen-4-ylcarbonylamino)-4-ethylhexanoic acid (0.032 g).

### Example N

### Synthesis of 2(RS)-(2′-chlorobiphen-4-ylcarbonylamino)-3-thiazol-2-ylpropionic acid methyl ester

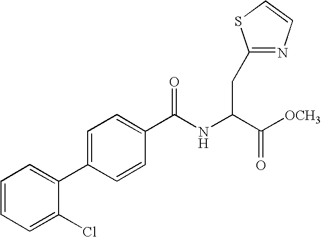 Step 1

To a solution of 2(RS)-amino-3-thiazol-2-ylpropionic acid (100 mg, 0.58 mmol) in the mixture of methanol (1 mL) and benzene (5 mL), was added (trimethyl)diazomethane (2 M solution in hexane, 0.76 ml) at room temperature. After 2 h, the solvent was removed under vacuum and 2(RS)-amino-3-thiazol-2-ylpropionic acid methyl ester was used in the next reaction without further purification.

Step 2

To a stirred solution of 2(RS)-amino-3-thiazol-2-ylpropionic acid methyl ester in methylene chloride (5 mL) was added 2′-chlorobiphenyl-4-carboxylic acid (132 mg, 0.57 mmol), HOBt (105 mg, 0.68 mmol), and then EDC (165 mg, 0.86 mmol) and N-methylmorpholine (0.3 mL) at room temperature. After stirring for 14 h, the reaction mixture was extracted with ethyl acetate. The organic layer was washed with saturated NaHCO₃, brine, dried with MgSO₄ and concentrated to yield 2(RS)-(2′-chlorobiphen-4-carbonylamino)-3-thiazol-2-ylpropionic acid methyl ester.

### Example O

### Synthesis of 2(S)-[(2′-chlorobiphen-4-ylcarbonyl)amino]-4(S)-phenylpentanoic acid

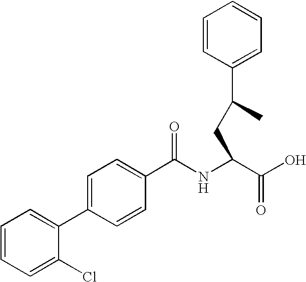

2(S)-(2′-Chlorobiphen-4-carbonylamino)-4(S)-phenylpentanoic acid methyl ester (275 mg, 0.65 mmol) was treated with methanol (2.5 mL) and 2N LiOH (0.65 mL) and allowed to stir at room temperature for 4 h. The reaction mixture was acidified with aqueous HCl and extracted with ethyl acetate, dried over magnesium sulfate and concentrated to give 231 mg (0.53 mmol, 87%) of 2(S)-(2′-chlorobiphen-4-carbonylamino)-4(S)-phenylpentanoic acid.

### Example 1

### Synthesis of 6-(2-chlorophenyl)-N-[1(S)-(cyanomethylcarbamoyl)-2-(2,6-difluorophenyl)ethyl]nicotinamide (table 1, compound 3)

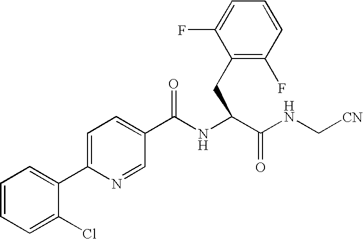

2(S)-[2-(2-Chlorophenyl)pyridin-5-ylcarbonylamino]-3-(2,6-difluorophenyl)propionic acid (0.480 mmol) (prepared as described in Example K above) was dissolved in acetonitrile (2 mL). To this solution was added 2-aminoacetonitrile (0.624 mmol), HBTU (0.624 mmol) and finally NMM (1.92 mmol). The reaction mixture was stirred at ambient temperature overnight. Saturated ammonium chloride and ethyl acetate were added, followed by stirring for an additional 20 minutes. Separation of layers, washing of the organic layer with saturated bicarbonate, saturated sodium chloride, drying over magnesium sulfate, and finally flash chromatograpy (ethyl acetatelhexane as eluent) afforded the title compound (0.352 mmol) as a white solid.

H¹ NMR (DMSO-d₆): δ 9.13 (1H, d, J=8.4 Hz), 9.02 (1H, m), 8.63 (1H, t, J=5.6 Hz), 8.20 (1H, dd, J=2.4, 8.0 Hz), 7.77 (1H, d, J=8.8 Hz), 7.59 (2H m), 7.48 (2H, m), 7.28 (1H, m), 7.03 (2H, m), 4.75 (1H, m), 4.12 (2H, d, J=5.6 Hz). MS 454.1 (M−1).

Proceeding as described in Example 1 above but substituting 2(S)-[2-(2-chlorophenyl)-pyridin-5-ylcarbonylamino]-3-(2,6-difluorophenyl)propionic acid with 1-(2′,3-dichlorobiphen-4-ylcarbonylamino)cycloheptanecarboxylic acid provided 2′,3-dichlorobiphenyl-4-carboxylic acid[1-(cyanomethylcarbamoyl)cycloheptyl]amide (table 3, compound 1).

H¹ NMR (DMSO-d₆): δ 8.49 (1H, s), 8.25 (1H, m), 8.13 (1H, d, J=7.4 Hz), 7.60 (1H, d, J=7.4 Hz), 7.50-7.28 (1H, m), 4.05 (2H, d, J=3.4 Hz), 2.10 (4H, m), 1.50 (8H, m). MS: 444.4 (M+1), 467.1 (M+Na).

Proceeding as described in Example 1 above but substituting 2(S)-[2-(2-chlorophenyl)-pyridin-5-ylcarbonylamino]-3-(2,6-difluorophenyl)propionic acid with 2(S)-(2′-chlorobiphen-4-ylcarbonylamino)-3-(2,6-difluorophenyl)propionic acid provided 2′-chlorobiphenyl-4-carboxylic acid[1(S)-(cyanomethylcarbamoyl)-2-(2,6-difluorophenyl)ethyl]amide (table 1, compound 1).

¹HNMR (DMSO-d₆): δ 8.85 (1H, d, J=8.4 Hz, NH), 8.63 (1H, t, J=5.6 Hz, NH), 7.85 (2H, d, J=8.4 Hz), 7.7-7.5 (1H, m), 7.45 (2H, d, J=8 Hz), 7.45-7.4 (3H, m), 7.26(1H, m), 6.984 (2H, m), 4.73 (1H, m), 4.09(2H, d, J=5.6 Hz), 3.3 (1H, m), 3.06 (1H, m). MS: 452.0(M−1), 454.0(M+1), 476.2(M+Na).

Proceeding as described in Example 1 above but substituting 2(S)-[2-(2-chlorophenyl)-pyridin-5-ylcarbonylamino]-3-(2,6-difluorophenyl)propionic acid with 2-(2′-chlorobiphen-4-carbonylamino)-4-phenylbutyric acid (prepared as described in example K, Steps 4 and 5 from commercially available homophenylalanine) provided 2′-chlorobiphenyl-4-carboxylic acid[1(RS)-(cyanomethylcarbamoyl)-(3-phenyl)propyl]amide (table 1, compound 15).

H¹ NMR(CDCl₃): δ 7.77 (1H, t, J=5.7 Hz), 7.73 (2H, d, J=8.4 Hz), 7.43 (3H, m), 7.05-7.30 (9H, m), 4.70 (1H, m), 4.05 (2H, m) 2.69 (2H, t, J=7.4 Hz), 2.15 (2H, m). MS: 430.2 (M−1), 454.2 (M+Na).

Proceeding as described in Example 1 above but substituting 2(S)-[2-(2-chlorophenyl)-pyridin-5-ylcarbonylamino]-3-(2,6-difluorophenyl)propionic acid with 2(RS)-(2′-chlorobiphen-4-carbonylamino)-4-methyl-4-phenylpentanoic acid (prepared as described in Example K, Steps 4 and 5 from 2-amino-4-methyl-4-phenylpentanoic acid which was prepared as in Examples D and C above) provided 2′-chlorobiphenyl-4-carboxylic acid[1(RS)-(cyanomethylcarbamoyl)-3-methyl-3-phenylbutyl]amide (table 1, compound 14).

H¹ NMR(CDCl₃): δ 7.43 (1H, m), 7.15-7.34 (12H, m), 7.08, (1H, m), 6.03 (1H, d, J=6.0 Hz), 4.74 (1H, m), 3.95 (2H, d, J=6.0 Hz), 2.18-2.42 (2H, m), 1.35 (3H, s), 1.32 (3H, s). MS: 458.2 (M−1), 460.2 (M+1), 482.2 (M+Na).

Proceeding as described in Example 1 above but substituting 2(S)-[2-(2-chlorophenyl)-pyridin-5-ylcarbonylamino]-3-(2,6-difluorophenyl)propionic acid with 2(S)-(2′-chlorobiphen-4-carbonylamino)-4-phenylpent-4-enoic acid (prepared as described in Example I) provided 2′-chlorobiphenyl-4-carboxylic acid[1(S)-(cyanomethylcarbamoyl)-3-phenylbut-3-enyl]amide(table 1, compound 16).

H¹ NMR(CDCl₃): δ 7.58 (2H, m), 7.40 (3H, m), 7.26 (7H, m), 7.10 (2H, m), 6.59, (1H, d, J=7.4 Hz), 5.39 (1H, m), 5.21 (1H, m), 4.59 (1H, m), 2.80 (2H, m). MS: 442.4 (M−1), 444.2 (M+1).

### Example 2

### Synthesis of 2′-chlorobiphenyl-4-carboxylic acid[1(S)-(cyanomethylcarbamoyl)-2-(2,6-difluoro-4-methoxyphenyl)ethyl]amide (table 1, compound 2)

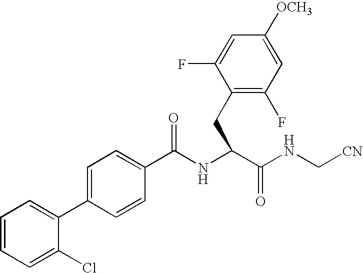 Step 1

To a solution of (S)-N-Cbz-(2,6-difluoro-4-methoxy)phenylalanine methyl ester (665 mg, 1.77 mmol) and Boc₂O (492 mg, 2.26 mmol) in methanol (25 mL) was added 5% Pd—C (120 mg). The system was flushed with H₂ and hydrogenated at 40 psi overnight at room temperature. The reaction mixture was filtered through a short pad of Celite, concentrated in vacuo, and purified by flash chromatography on silica gel (eluted with 1:3 EtOAc/hexanes) to yield 2(S)-tert-butoxycarbonylamino-3-(2,6-difluoro-4-methoxyphenyl)propionic acid methyl ester (528 mg) as a white solid.

Step 2

To a stirred solution of 2(S)-tert-butoxycarbonylamino-3-(2,6-difluoro-4-methoxyphenyl)-propionic acid methyl ester (510 mg, 1.48 mmol) in methanol (6 mL) at room temperature was added aqueous 1N KOH solution (1.90 mL). After 3 h, the reaction mixture was concentrated, diluted with water (5 mL), acidified with 1N HCl (pH=ca. 3), and then extracted with ethyl acetate. The combined organic layers were dried over MgSO₄, and concentrated to give 2(S)-2-tert-butoxycarbonylamino-3-(2,6-difluoro-4-methoxyphenyl)-propionic acid (490 mg), which was used directly without further purification.

Step 3

To a stirred solution of 2(S)-tert-butoxycarbonylamino-3-(2,6-difluoro-4-methoxyphenyl)-propionic acid methyl ester (387 mg, 1.17 mmol) in dichloromethane (10 mL) at room temperature was added aminoacetonitrile hydrochloride (130 mg, 1.40 mmol) followed by 1-hydroxybenzotriazole (232 mg, 1.52 mmol) and N-methylmorpholine (0.39 mL, 3.50 mmol). N-(3-dimethylaminopropyl)-N′-ethylcarbodiimide hydrochloride (338 mg, 1.75 mmol) was then as a solid. After 3 h, the reaction mixture was diluted with dichloromethane, washed with water. The organic layer was dried over MgSO₄, filtered, and concentrated. Purification by flash chromatography on silica gel(eluted with 1:1 EtOAc/hexanes) afforded[1(S)-(cyanomethyl-carbamoyl)-2-(2,6-difluoro-4-methoxyphenyl)ethyl]carbamic acid tert-butyl ester (365 mg) as a white solid.

Step 4

To a stirred solution of[1(S)-(cyanomethylcarbamoyl)-2-(2,6-difluoro-4-methoxyphenyl)-ethyl]carbamic acid tert-butyl ester (354 mg, 0.96 mmol) in THF (1 mL) at room temperature was added methanesulfonic acid (0.25 mL, 3.84 mmol) dropwise, and the reaction mixture was stirred at the same temperature for 4 h. The reaction mixture was quenched with aqueous saturated NaHCO₃ solution, extracted with ethyl acetate, dried, and concentrated in vacuo to give 2(S)-amino-N-cyanomethyl-3-(2,6-difluoro-4-methoxyphenyl)-propionamide (247 mg) as a white solid, which was used directly without further purification.

Step 5

To a stirred solution of 2(S)-amino-N-cyanomethyl-3-(2,6-difluoro-4-methoxyphenyl)-propionamide (115 mg, 0.428 mmol) in dichloromethane (5 mL) at room temperature was added 2′-chlorobiphenyl-4-carboxylic acid (100 mg, 0.428 mmol) followed by 1-hydroxybenzotriazole (79 mg, 0.514 mmol) and N-methylmorpholine (47 μL, 0.428 mmol). N-(3-dimethylaminopropyl)-N′-ethylcarbodiimide hydrochloride (107 mg, 0.55 6 mmol) was then as a solid. After 3 h, the reaction mixture was diluted with dichloromethane, washed with water, and organic layer was dried over MgSO₄, filtered, and concentrated. Purification by flash chromatography on silica gel (eluted with 1:1 EtOAc/hexanes) afforded the title compound (135 mg) as a white solid. ¹H NMR (400 MHz, CDCl₃+trace CD₃OD): δ 7.76 (2H, d, J=6.0 Hz), 7.46 (2H, d, J=6.0 Hz), 7.44-7.26 (m, 4H), 6.41 (2H, d, J_{HF}=9.6 Hz), 4.84 (1H, m), 4.10 (2H, m), 3.71 (3H, m), 3.20 (1H, dd, J=14.0, 5.6 Hz), 3.09 (1H, dd, J=14.0, 7.6 Hz). MS: 484.1(MH⁺), 486.2(MH⁺+2).

Proceeding as described in Example 2 above, but using 6-(2-chlorophenyl)nicotinic acid instead of 2′-chlorobiphenyl-4-carboxylic acid, provided 6-(2-chlorophenyl)-N-[1(S)-(cyanomethylcarbamoyl)-2-(2,6-difluoro-4-methoxyphenyl)ethyl]nicotinamide as a white solid in 79% yield (table 1, compound 30).

¹H NMR (400 MHz, CDCl₃+trace CD₃OD): δ 8.92 (1H, d, J=2.0 Hz), 8.08 (1H, dd, J=8.0, 2.0 Hz), 7.96 (1H, d, J=8.0 Hz), 7.65(1H, d, J=8.4 Hz), 7,46(1H), 7.40 (1H, m), 7.30 (2H, m), 6.37(2H, d, J_{HF}=9.6 Hz), 4.77(1H, m), 4.10 (2H, m), 3.67 (3H,m), 3.17(1H, dd, J=14.0, 5.6 Hz), 3.03(1H, dd, J=14.0, 8.0 Hz). MS: 485.0(MH⁺), 487.0(MH⁺+2).

Proceeding as described in Example 2 above, but substituting 2(S)-tert-butoxycarbonylamino-3-(2,6-difluoro-4-methoxyphenyl)-propionic acid with 2(S)-tert-butoxycarbonylamino-3-(2,6-difluorophenyl)-propionic acid, 2′-chlorobiphenyl-4-carboxylic acid with 6-(2-chlorophenyl)nicotinic acid, and aminoacetonitrile hydrochloride with 4-amino-1-ethyl-piperidine-4-carbonitrile provided 6-(2-chlorophenyl)-N-[1(S)-(4-cyano-1-ethylpiperidin-4-ylcarbamoyl)-2-(2,6-difluorophenyl)ethyl]nicotinamide (table 2, compound 6).

H¹ NMR(CDCl₃): δ 9.10 (1H, d, J=3.2 Hz), 8.10 (1H, dd, J=7.2, 2.2 Hz), 7.75 (1H, d, J=7.2 Hz), 7.60 (2H, m), 7.48 (2H, m), 7.39 (2H m), 7.21 (1H, m), 7.13 (8H, m), 6.80 (2H, t, J=7.4 Hz), 4.87 (1H, m), 3.72 (1H, m), 3.35 (2H, m), 3.15 (1H, m), 1.4-1.5 (6H, m), 1.25 (2H, m) 553.4 (M+1).

Proceeding as described in Example 2 above, but substituting 2(S)-tert-butoxycarbonylamino-3-(2,6-difluoro-4-methoxyphenyl)propionic acid with 2(S)-tert-butoxycarbonylamino-3-(2,6-difluorophenyl)propionic acid, 2′-chlorobiphenyl-4-carboxylic acid with 6-(2-chlorophenyl)nicotinic acid and aminoacetonitrile hydrochloride with 4-aminotetrahydrothiopyran-4-carbonitrile provided 6-(2-chlorophenyl)-N-[1(S)-(4-cyanotetrahydro-thiopyran-4-ylcarbamoyl)-2-(2,6-difluorophenyl)ethyl]nicotinamide (table 2, compound 11).

Proceeding as described in Example 2 above, but substituting 2(S)-tert-butoxycarbonylamino-3-(2,6-difluoro-4-methoxyphenyl)propionic acid with 2(S)-tert-butoxycarbonylamino-4(S)-phenylpentanoic acid, and aminoacetonitrile hydrochloride with 4-amino-1-ethylpiperidine-4-carbonitrile provided 2′-chlorobiphenyl-4-carboxylic acid[1(S)-(4-cyano-1-ethylpiperidin-4-ylcarbamoyl)-3(S)-phenylbutyl]amide(table 2, compound 14).

H¹ NMR (DMSO-d₆): δ 8.660 (1H, d, J=8.0 Hz), 8.526 (1H, s), 7.960 (2H, d, J=8.0 Hz), 7.56-7.59 (1H, m), 7.521 (2H, d, J=8.2 Hz), 7.434 (2H, m), 7.24-7.30 (3H, m), 7.14-7.18 (1H, m), 4.624 (1H, m), 2.83-2.92 (1H, m), 2.660 (2H, m), 2.316(2H, q, J=5.8 Hz), 2.217 (4H, m), 2.00-2.19 (1H, m), 1.80-1.95 (3H, m), 1.231 (3H, d, J=6.4 Hz), 0.971 (3H, t, J=5.8 Hz). MS: 541.2 (M−1), 543.1 (M+1), 565.2 (M+Na).

Proceeding as described in Example 2 step 5 above, but substituting 2(S)-amino-N-cyanomethyl-3-(2,6-difluoro-4-methoxyphenyl)propionamide with 2(S)-amino-4-methyl-pentanoic acid cyanomethyl-amide and 2′-chlorobiphenyl-4-carboxylic acid with 3,5-di(2-methoxyphenyl)benzoic acid provided 2,2″-dimethoxy-[1,1′;3′,1″]terphenyl-5′-carboxylic acid[1(S)-(cyanomethyl-carbamoyl)-3-methylbutyl]-amide (table 1, compound 6).

¹H-NMR(DMSO-d₆): δ 8.72˜8.60 (2H, m), 7.93(2H, d, J=1.6 Hz), 7.70 (1H, t, J=1.6 Hz), 7.42˜7.32 (4H, m), 7.16˜7.00 (4H, m), 4.53 (1H, m), 4.12 (2H, d, J=6 Hz), 3.76 (6H, s), 1.80˜1.50 (3H, m), 0.89(6H, d, J=3.2 Hz). MS: 486 (M+1).

Proceeding as described in Example 2 step 5 above, but substituting 2(S)-amino-N-cyanomethyl-3-(2,6-difluoro-4-methoxyphenyl)propionamide with 2(S)-amino-4-methyl-pentanoic acid cyanomethyl-amide and 2′-chlorobiphenyl-4-carboxylic acid with 2,3-di-(2-methylphenyl)-thiophene-5-carboxylic acid provided 2,3-di-(2-methylphenyl)thiophene-5-carboxylic acid[1(S)-(cyanomethyl-carbamoyl)-3-methylbutyl]-amide (table 1, compound 7).

¹H-NMR (DMSO-d₆): δ 8.72˜8.60 (2H, m), 7.95 (1H, s), 7.25˜6.90 (8H, m), 4.46 (1H, m), 4.12 (2H, d, J=5.6 Hz), 2.12 (3H, s), 1.97 (3H, s), 1.80˜1.40 (3H, m), 0.89(6H, d, J=15.6 Hz). MS: 460 (M+1).

Proceeding as described in Example 2 step 5 above, but substituting 2(S)-amino-N-cyanomethyl-3-(2,6-difluoro-4-methoxyphenyl)propionamide with 2(S)-amino-4-methyl-pentanoic acid cyanomethyl-amide and 2′-chlorobiphenyl-4-carboxylic acid with 1-methyl-3-trifluoromethyl-1H-thieno[2,3-c]pyrazole-5-carboxylic acid provided 1-methyl-3-trifluoromethyl-1H-thieno[2,3-c]pyrazole-5-carboxylic acid[1(S)-(cyanomethylcarbamoyl)-3-methylbutyl]-amide (table 1, compund 22).

¹H NMR (DMSO-d₆): δ 8.90˜8.70 (2H, m), 8.10 (1H, s), 7.25˜6.90 (8H, m), 4.45 (1H, m), 4.12 (2H, d, J=5.6 Hz), 4.06 (3H, s), 1.80˜1.40 (3H, m), 0.89 (6H, d, J=20 Hz). MS: 402 (M+1).

Proceeding as described in Example 2 step 5 above, but substituting 2(S)-amino-N-cyanomethyl-3-(2,6-difluoro-4-methoxyphenyl)propionamide with 2(S)-amino-4-methylpentanoic acid cyanomethylamide and 2′-chlorobiphenyl-4-carboxylic acid with 3-(2-chlorophenyl)-isoxazole-5-carboxylic acid provided 3-(2-chlorophenyl)isoxazole-5-carboxylic acid[1(S)-(cyanomethyl-carbamoyl)-3-methylbutyl]-amide (table 1, compound 8).

¹H-NMR (DMSO-d₆): δ 9.21 (1H, d, J=8.4 Hz), 8.82(1H, t, J=5.6 Hz), 7.80˜7.40 (6H, m), 4.48 (1H, m), 4.13 (2H, d, J=5.2 Hz), 1.80˜1.40 (3H, m), 0.89 (6H, d, J=17.2 Hz). MS: 375 (M+1).

Proceeding as described in Example 2 step 5 above, but substituting 2(S)-amino-N-cyanomethyl-3-(2,6-difluoro-4-methoxyphenyl)propionamide with 2(S)-amino-N-cyanomethyl-3-phenylpropionamide and 2′-chlorobiphenyl-4-carboxylic acid with 2,3-diphenylthiophen-5-carboxylic acid provided 4,5-diphenylthiophene-2-carboxylic acid[1(S)-(cyanomethyl-carbamoyl) 2-phenylethyl]-amide (table 1, compound 26).

¹H-NMR (DMSO-d₆): δ 8.85˜8.78 (2H, m), 8.01(1H, s), 7.40˜7.10 (15H, m), 4.66 (1H, m), 4.15 (2H, d, J=5.6 Hz), 3.20˜2.90 (2H, m). MS: 466 (M+1).

Proceeding as described in Example 2 step 5 above, but substituting 2(S)-amino-N-cyanomethyl-3-(2,6-difluoro-4-methoxyphenyl)propionamide with 2(S)-amino-N-cyanomethyl-3-(2,6-difluorophenyl)propionamide and 2′-chlorobiphenyl-4-carboxylic acid with 1-methyl-3-trifluoromethyl-1H-thieno[2,3-c]pyrazole-5-carboxylic acid provided 1-methyl-3-trifluoromethyl-1H-thieno[2,3-c]pyrazole-5-carboxylic acid[1(S)-(cyanomethylcarbamoyl)-2-(2,6-difluorophenyl)-ethyl]-amide (table 1, compound 23).

¹H-NMR (DMSO-d₆): δ 9.11 (1H, d, J=8.4 Hz), 8.75 (1H, t, J=5.6 Hz), 8.02 (1H, s), 7.26 (1H, m), 6.98 (2H, t, J=7.6 Hz), 4.67 (1H, m), 4.09 (2H, d, J=5.6 Hz), 4.05 (3H, s), 3.20˜3.40 (1H, m), 3.10˜2.90 (1H, m). MS: 472 (M+1).

### Example 3

### Synthesis of 2′-chlorobiphenyl-4-carboxylic acid[1(S)-[4-cyano-1-(2,2,2-trifluoroethyl)-piperidin-4-ylcarbamoyl]-(2,6-difluorophenyl)ethyl]amide (table 2, compound 3)

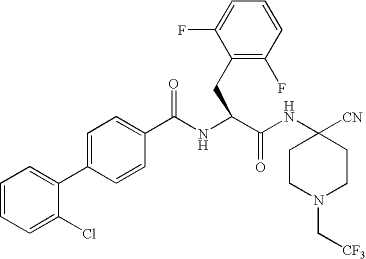 Step 1

In a solution of 1,4-dioxa-8-aza-spiro[4.5]decane (14.3 g, 100 mmol) in CH₂Cl₂ (200 mL) was added Et₃N (15.2 g, 150 mmol), DMAP (30 mg) and trifloroacetic acid anhydride (25.2 g, 150 mmol) at 0° C., then allowed to warm-up to room temperature and stirred for 12 h. The reaction mixture was quenched with water and washed with 1N HCl and brine, dried with MgSO₄. Removal of the solvent, yielded 1-(1,4-dioxa-8-aza-spiro[4.5]dec-8-yl)-2,2,2-trifluoroethanone (35 g). The crude product was used in the next reaction.

Step 2

In the solution of 1-(1,4-dioxa-8-aza-spiro[4.5]dec-8-yl)-2,2,2trifluoroethanone (20 g, 83.7 mmol) in THF, borane-methyl sulfide complex (83.7 ml, 2M solution in THF) was added at 0° C. After refluxing the reaction mixture for 12 h, it was cooled and quenched with MeOH. After removal of THF, the residue was extracted with ethyl acetate and washed with brine, dried with MgSO₄. After concentration of the organics 8-(2,2,2-trifluoroethyl)-1,4-dioxa-8-aza-spiro[4.5]decane (19 g) was obtained.

Step 3

8-(2,2,2-Trifluoroethyl)-1,4-dioxa-8-aza-spiro[4.5]decane (3.7 g, 16 mmol) was added to a solution of 5% HCl (45 mL) and acetone (8 mL). After reflux for 12 h, the solvent was removed, to give crude 1-(2,2,2-trifluoroethyl)piperidin-4-one hydrochloride which was used in the next reaction.

Step 4

A solution of ammonium chloride (3.2 g, 60 mmol) and potassium cyanide (2.94 g, 60 mmol) was prepared in water (25 mL) and 1-(2,2,2-trifluoroethyl)-piperidin-4-one hydrochloride (3.5 g, 15 mmol) was added and the reaction mixture was stirred for 2 days. The solution was then brought to pH 11 with sodium carbonate and the reaction mixture was extracted with ethyl acetate. After drying over Na₂SO₄, the solvent was removed to yield a mixture of 4-hydroxy-1-(2,2,2-trifluoroethyl)piperidine-4-carbonitrile and 4-amino-1-(2,2,2-trifluoroethyl)piperidine-4-carbonitrile. This mixture was then treated with 7N ammonia solution in MeOH for 12 h at room temperature. After removal of the solvent, the residue was dissolved in ethyl ether and treated with 4N HCl solution in dioxane. The solids were filtered and dried under vacuum, to yield 4-amino-1-(2,2,2-trifluoroethyl)piperidine-4-carbonitrile hydrochloride (2.5 g).

Step 5

4-Amino-1-(2,2,2-trifluoroethyl)-piperidine-4-carbonitrile hydrochloride salt was reacted with (2S)-(2′-chlorobiphen-4-ylcarbonylamino)-3-(2,6-difluorophenyl)propionic acid as described in Example 2 above to provide the title compound.

¹HNMR (DMSO-d₆): δ 8.725 (1H, d, J=8.4 Hz, NH), 8.49 (1H, s, NH), 7.88 (2H, d, J=8.4 Hz), 7.6-7.5 (1H, m), 7.50 (2H, d, J=8.4 Hz), 7.45-7.4 (3H, m), 7.3 (1H, d,d,d), 7.02 (2H, d,d,d), 4.77 (1H, d,d,d, J=7.6 Hz), 3.25-3.2 (3H, m), 3.2-3.0 (1H, d,d), 2.85-2.7 (2H, m), 2.65-2.5 (2H, d,d), 2.2-2.1 (2H, d), 1.85-1.65 (2H, m). MS: 603.3 (M−1), 605.4(M+1), 627.3(M+Na).

Proceeding as described in Example 3, Steps 1-4 above, 4-amino-1-ethylpiperidine-4-carbonitrile and 4-amino-1-cyclopropylpiperidine-4-carbonitrile were prepared.

Proceeding as described above, but substituting 4-amino-1-(2,2,2-trifluoroethyl)-piperidine-4-carbonitrile with 4-amino-1-ethyl-piperidine-4-carbonitrile provided 2′-chlorobiphenyl-4-carboxylic acid[1(S)-[4-cyano-1-ethylpiperidin-4-ylcarbamoyl]-2-(2,6-difluorophenyl)ethyl]amide (table 2, compound 2).

¹HNMR (DMSO-d₆): δ 8.72 (1H, d, J=8.0 Hz, NH), 8.45 (1H, s, NH), 7.88 (2H, d, J=8.4 Hz), 7.6-7.5 (1H, m), 7.50 (2H, d, J=8.4 Hz), 7.45-7.4 (3H, m), 7.3 (1H, ddd), 7.02 (2H, ddd), 4.768 (1H, ddd, J=7.6 Hz), 3.25-3.2(1H, dd), 3.1-3.0 (1H, d,d), 2.65-2.52 (2H, m), 2.28 (2H, q, J=7.2 Hz), 2.2-2.1 (4H, m), 1.8-1.6 (2H, m), 0.952 (3H, t, J=7.2 Hz). MS: 549.3 (M−1), 451.3 (M+1), 573.4(M+Na).

Proceeding as described above, but substituting 4-amino-1-(2,2,2-trifluoroethyl)-piperidine-4-carbonitrile with 4-aminotetrahydrothiopyran-4-carbonnitrile provided 2′-chlorobiphenyl-4-carboxylic acid[1(S)-[4-cyanotetrahydrothiopyran-4-ylcarbamoyl]-2-(2,6-difluorophenyl)-ethyl]amide.

Proceeding as described above, but substituting 4-amino-1-(2,2,2-trifluoroethyl)-piperidine-4-carbonitrile with 4-amino-1-cyclopropylpiperidine-4-carbonitrile provided 2′-chlorobiphenyl-4-carboxylic acid[1(S)-[4-cyano-1-cyclopropylpiperidin-4-ylcarbamoyl]-2-(2,6-difluorophenyl)-ethyl]amide. ¹HNMR (DMSO-d₆): δ 8.72(1H, d, J=8.4 Hz, NH), 8.454 (1H, s, NH), 7.88 (2H, d, J=8.0 Hz), 7.6-7.5 (1H, m), 7.50 (2H, d, J=8.0 Hz), 7.45-7.4 (3H, m), 7.3 (1H, d,d,d), 7.02 (2H, d,d,d), 4.765 (1H, d,d,d, J=8.0 Hz), 3.25-3.2 (1H, d,d), 3.1-3.0 (1H, d,d), 2.65-2.52 (2H, m), 2.45-2.3 (2H, m), 2.2-2.05 (2H, m), 1.8-1.5 (3H, m), 0.388 (2H, m), 0.264 (2H, m). MS: 561.2(M−1), 563.0(M+1), 585.4(M+Na) (table 2, compound 4).

### Example 4

### Synthesis of 4-chloro-3-{5-[1(S)-(cyanomethylcarbamoyl)-3-methylbutylcarbamoyl]-pyridin-2-yl}-benzoic acid (table 1, compound 20)

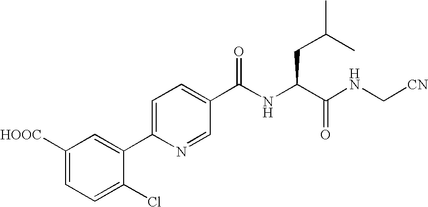 Step 1

To a solution of N-[1(S)-(cyanomethylcarbamoyl)-3-methylbutyl]-6-hydroxy-nicotinamide (1.29 g, 4.4 mmol) (prepared as described in Example 2 above) and 2,6-lutidine (2.4 g, 22.2 mmol) in dichloromethane (50 mL), was added trifluoromethanesulfonic anhydride (1.89 g, 6.7 mmol) at 0° C. After 2 h, the reaction mixture was extracted with dichloromethane. The organic layer was washed with 1 N HCl, sat. NaHCO₃, and brine. Dried with MgSO₄ and concentrated to yield trifluoromethanesulfonic acid 5-[1(S)-(cyanomethylcarbamoyl)-3-methylbutylcarbamoyl]-pyridin-2-yl ester. This triflate was used in the next step without further purification.

Step 2

3-Bromo-4-chloro-benzoic acid methyl ester (249 mg, 1 mmol), prepared from 3-bromo-4-chloro-benzoic acid and (trimethylsilyl)diazomethane (J. Org. Chem., 1996, 61, 8940-8948), bis(pinacolato)diboron (304 mg, 1.2 mmol), [1,1′-bis(diphenylphosphino)ferrocene]dichloropalladium (II), complex with dichloromethane (41 mg, 0.05 mmol), potassium acetate (294 mg, 3 mmol) and dioxane (2 mL) were added in a 5 ml microwave vial. The reaction mixture was heated at 120° C. for 5 min., in microwave reactor. The reaction mixture was extracted with ethyl acetate. The organic layer was washed with sat. NaHCO₃, and brine, dried with MgSO₄ and concentrated. The crude 4-chloro-3-(4,4,5,5-tetramethyl-[1,3,2]dioxaborolan-2-yl)-benzoic acid methyl ester was used in the next step without further purification.

Step 3

Trifluoromethanesulfonic acid 5-[1(S)-(cyanomethylcarbamoyl)-3-methylbutylcarbamoyl]-pyridin-2-yl ester (422 mg, 1 mmol), 4-chloro-3-(4,4,5,5-tetramethyl-[1,3,2]dioxaborolan-2-yl)-benzoic acid methyl ester (296 mg, 1 mmol), tetrakis(triphenylphosphine)-palladium(0) (57 mg, 0.05 mmol), potassium carbonate (690 mg, 5 mmol) and DMF (3 mL) were added in a 5 mL microwave vial. The reaction mixture was heated at 150° C. for 5 min in microwave reactor. The reaction mixture was extracted with ethyl acetate. The organic layer was washed with sat. NaHCO₃, and brine. Dried with MgSO₄ and concentrated. The residue was purified by chromatography (30%-40% EtOAc/Hexane) to afford 4-chloro-3-{5-[1(S)-(cyanomethyl-carbamoyl)-3-methyl-butylcarbamoyl]-pyridin-2-yl}-benzoic acid methyl ester (100 mg).

Step 4

To a solution of 4-chloro-3-{5-[1(S)-(cyanomethylcarbamoyl)-3-methylbutylcarbamoyl]-pyridin-2-yl}benzoic acid methyl ester (26 mg, 0.058 mmol) in DMF, was added 1.0 M tetrabutylammonium fluoride in THF (0.3 mL, 0.3 mmol) at room temperature. After stirring at room temperature overnight, the solvent was removed under vacuum. The residue was purified by prep-HPLC to afford the title compound (20 mg).

### Example 5

### Synthesis of 6-(2-chlorophenyl)-N-[1(S)-(cyanomethylcarbamoyl)-3-methylbutyl]-1-oxynicotinamide (table 1, compound 31)

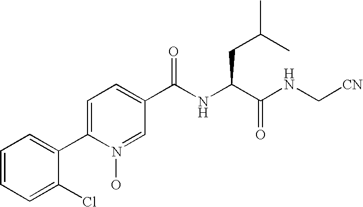

To a solution of 6-(2-chlorophenyl)-N-[1(S)-(cyanomethyl-carbamoyl)-3-methylbutyl]-nicotinamide (76 mg, 0.2 mmol) (prepared as described in Example 2 above) in dichloromethane (5 mL), was added 3-chloroperpoxybenzoic acid (41 mg, 0.24 mmol) at room temperature. After stirring at room temperature overnight, the solvent was removed under vacuum. The residue was purified by chromatography (10% MeOH/EtOAc) to afford the title compound (20 mg).

### Example 6

### Synthesis of 2-chloro-4′-[1(S)-(cyanomethylcarbamoyl)-3-methylbutylcarbamoyl]-biphenyl-4-carboxylic acid (table 1, compound 11)

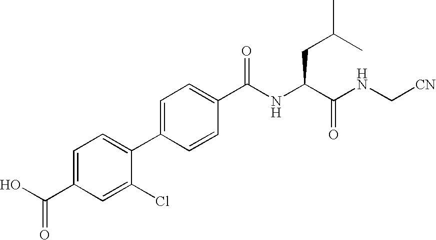 Step 1

To the mixture of {4-[1(S)-(cyanomethylcarbamoyl)-3-methylbutylcarbamoyl]-phenyl}-boronic acid (317 mg, 1 mmol) (prepared by reacting N-tert-butoxycarbonyl-L-leucine monohydrate under the conditions described in Example 2, Step 3 above to provide[1(S)-(cyanomethylcarbamoyl)-3-methylbutyl]carbamic acid tert-butyl ester. Removal of the tert-butoxycarbonyl group under the conditions described in Example 2, Step 4 above provided the corresponding free amine compound which was then reacted with 4-carboxyphenylboronic acid as described in Example 2, Step 3 above except for the addition of aminoacetonitrile), 3-chloro-4-trifluoromethanesulfonyloxy-benzoic acid methyl ester (318.5 mg, 1 mmol) (prepared as described in Example H), [1,1′-bis(diphenylphosphino)-ferrocene]-dichloropalladium (II) complex with dichloromethane (1:1) (40 mg, 0.05 mmol) in acetonitrile (5 mL), was added 2M sodium carbonate aqueous solution (0.1 ml, 0.2 mmol), then bubbled with N₂ for few minutes. The vial was placed in a microwave reactor for 10 min., under 160 degrees. The mixture was diluted with 200 ml ethyl acetate, then washed with sodium bicarbonate and brine, dried over magnesium sulfate, and concentrated under vacuum. The residue was purified by column using 1:1 ethyl acetate and hexane to give 150 mg the product 2-chloro-4′-[1(S)-(cyanomethyl-carbamoyl)-3-methyl-butylcarbamoyl]-biphenyl-4-carboxylic acid methyl ester as white solid.

Step 2

To a solution of 2-chloro-4′-[1(S)-(cyanomethylcarbamoyl)-3-methylbutylcarbamoyl]-biphenyl-4-carboxylic acid methyl ester (88 mg, 0.2 mmol) in NN-dimethylformide (5 mL) was added 1 ml tetrabutylammonium fluoride (1 M in TBF, 1 mmol), and the solution was stirred at room temperature for 24 h. The reaction mixture was diluted with 200 mL ethyl acetate, washed with water and brine, dried over magnesium sulfate, then concentrated to give 67 mg of the title compound as a white solid.

¹H-NMR(DMSO-d₆): δ 8.72˜8.66 (2H, m), 8.04˜7.98 (3H, m), 7.95 (1H, d, J=8 Hz), 7.60˜7.54 (3H, m), 4.52˜4.48(1H, m), 4.12(2H, d, J=5.6 Hz), 1.80˜1.50 (3H, m), 0.89 (6H, d, J=3.2 Hz). MS: 428 (M+1).

### Example 7

### Synthesis of 4-{4-[1(S)-(cyanomethylcarbamoyl)-3-methylbutylcarbamoyl]phenyl}-5-methyl-thiophene-2-carboxylic acid (table 1, compound 25)

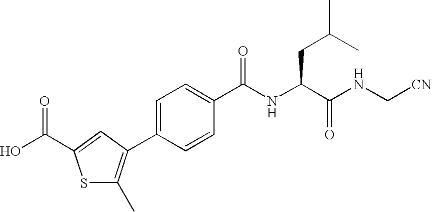

Prepared as described in Example 6 step 1 above, but substituting 3-chloro-4-trifluoromethanesulfonyloxybenzoic acid methyl ester with 2-methyl-3-bromo-thiopene-5-carboxylic acid to provide the title compound.

¹H-NMR (DMSO-d₆): δ 8.68 (1H, t, J=5.6 Hz), 8.62 (1H, d, J=8.4 Hz), 7.99˜7.55 (4H, m), 7.72 (1H, s), 4.51(1H, m), 4.12 (2H, d, J=8.4 Hz), 2.53 (3H, s), 1.80˜1.50 (3H, m), 0.89 (6H, d, J=3.2 Hz). MS: 414.3 (M+1).

Proceeding as described in Example 7 above, but substituting 2-methyl-3-bromothiophenyl-5-carboxylic acid with 3-bromo-4-methylbenzoic acid provided 4′-[1(S)-(cyanomethylcarbamoyl)-3-methylbutylcarbamoyl]-6-methylbiphenyl-3-carboxylic acid (table 1, compound 8).

¹H-NMR (DMSO-d₆): δ 8.74˜8.60 (2H, m), 8.10˜7.30 (7H, m), 4.52(1H, m), 4.12 (2H, d, J=4.8 Hz), 2.29 (3H, s), 1.80˜1.50 (3H, m), 0.89 (6H, d, J=16.8 Hz). MS: 408 (M+1).

Proceeding as described in Example 7 above, but substituting 2-methyl-3-bromothiopenyl-5-carboxylic acid with 3-bromo-4-chlorobenzoic acid to provide 3-bromothiopenyl-5-carboxylic acid with 3-bromo-4-methyl-benzoic acid to provide 4′-[1(S)-(cyanomethylcarbamoyl)-3-methyl-butylcarbamoyl]-6-chloro-biphenyl-3-carboxylic acid (table 1, compound 10).

¹H-NMR (DMSO-d₆): δ 8.74˜8.60 (2H, m), 8.10˜7.45 (7H, m), 4.52 (1H, m), 4.12 (2H, d, J=5.2 Hz), 1.80˜1.50 (3H, m), 0.89 (6H, d, J=16.8 Hz). MS: 428 (M+1).

### Example 8

### Synthesis of 2-chloro-4′-[1(S)-(4-cyano-1-ethylpiperidin-4-ylcarbamoyl)-3(S)-phenylbutyl-carbamoyl]biphenyl-5-carboxylic acid (table 2, compound 12)

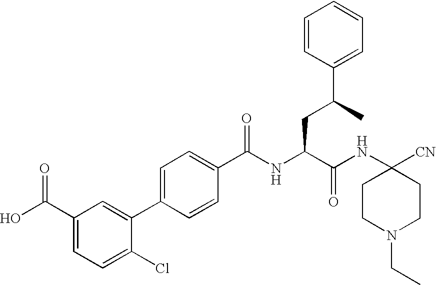

Proceeding as described in Example 6 step 2 above, but substituting 2-chloro-4′-[1(S)-(cyanomethylcarbamoyl)-3-methylbutylcarbamoyl]biphenyl-4-carboxylic acid methyl ester with 2-chloro-4′-[1(S)-(4-cyano-1-ethylpiperidin-4-ylcarbamoyl)-3(S)-phenylbutylcarbamoyl]biphenyl-5-carboxylic acid methyl ester which was prepared as described in Example 1 above, provided the title compound.

¹H-NMR (DMSO-d₆): δ 8.78 (1H, m), 8.04˜7.50 (7H, m), 7.30˜7.10 (5H, m), 4.58 (1H, m), 2.20˜1.80 (5H, m), 1.55 (1H, m), 1.40˜1.20 (10H,m), 0.92 (3H, t, J=7.6 Hz). MS: 587 (M+1).

Proceeding as described in Example 8 above, but substituting 4-amino-1-ethylpiperidine-4-carbonitrile with 4-aminotetrahydropyran-4-carbonitrile and 3-phenylbutylcarbamoyl with 2-(2,6-difluorophenyl)ethylcarbamoyl provided 6-chloro-4′-[1(S)-(4-cyanotetrahydropyran-4-ylcarbamoyl)-2-(2,6-difluorophenyl)ethylcarbamoyl]biphenyl-3-carboxylic acid (table 2, compound 15).

¹H-NMR (DMSO-d₆): δ8.79 (1H, d, J=7.8 Hz), 8.57 (1H, s), 8.00˜6.80 (10H, m), 4.77 (1H, m), 3.80˜3.60 (2H, m), 3.20˜3.00 (2H, m), 2.20˜2.00 (2H, m), 1.90˜1.60 (2H, m). MS: 568 (M+1).

Proceeding as described in Example 8 above, but substituting 4-amino-1-ethylpiperidine-4-carbonitrile with aminoacetonitrile provided 6-chloro-4′-[1(S)-(cyanomethyl-carbamoyl)-3(S)-phenyl-butylcarbamoyl]-biphenyl-3-carboxylic acid (table 1, compound 29).

¹H-NMR (DMSO-d₆): δ 8.76 (1H, d, J=4.4 Hz), 8.67 (1H, t, 5.6 Hz), 8.10˜7.10 (12H, m), 4.57 (1H, m), 4.12 (2H, d, J=5.6 Hz), 2.90˜2.70 (1H, m), 2.10˜2.90 (2H, m), 1.21 (3H, d, J=5.8 Hz). MS: 490 (M+1).

### Example 9

### Synthesis of 2′-chlorobiphenyl-4-carboxylic acid[1(S)-(4-cyano-1,1-dioxohexahydro-1λ⁶ thiopyran-4-ylcarbamoyl)-3(S)-phenylbutyl]amide (table 2, compound 13)

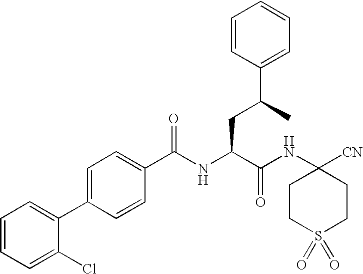

2′-Chlorobiphenyl-4-carboxylic acid[1(S)-(4-cyanotetrahydrothiopyran-4-ylcarbamoyl)-3(S)-phenylbutyl]-amide (prepared as described in Example 2 above) (120 mg, 0.22 mmol) was dissolved in methanol (2.4 mL) and treated with a 0.6 mL aqueous solution of OXONE (368 mg, 0.6 mmol) and stirred at room temperature for 2 h. Water (5 mL) was added and the reaction mixture was extracted with ethyl acetate, dried of magnesium sulfate and concentrated. The crude product was purified by silica gel chromatography (3:1 hexanes:ethyl acetate) and recrystallized from an ethyl acetate and hexane solution to give the title compound (57 mg).

¹H-NMR (DMSO-d₆): δ 8.748 (2H, s), 8.722 (1H, d, J=7.9 Hz), 7.95 (2H, d, J=9.2 Hz), 7.56-7.59 (1H, m), 7.522 (2H, d, J=8.2 Hz), 7.435 (2H, m), 7.285 (1H, d, J=1.9 Hz), 7.275 (2H, s), 7.14-7.19 (1H, m), 4.54-4.61 (1H, m), 3.15-3.4 (6H, m), 2.86-2.93 (1H, m), 2.56 (2H, m), 2.03-2.11 (1H, m), 1.90-1.98 (1H, m), 1.243 (3H, d, J=6.6 Hz). MS: 562.2 (M−1), 564.2 (M+1), 586.3 (M+Na).

Proceeding as described above following compounds were prepared:

6-(2-Chlorophenyl)-N-[1(S)-(4-cyano-1,1-dioxohexahydro-1λ⁶-thiopyran-4-ylcarbamoyl)-2-(2,6-difluorophenylethyl]nicotinamide (table 2, compound 7).

¹H-NMR (DMSO-d₆): δ 9.13 (1H, d, J=8.0 Hz), 9.02 (1H, m), 8.68 (1H, s), 8.20 (1H, dd, J=2.4, 8.0 Hz), 7.77 (1H, d, J=8.8 Hz), 7.59 (2H m), 7.48 (2H, m), 7.32 (1H, m), 7.03 (2H, m), 4.75 (1H, m), 3.25-3.05 (8H, m) 571.2 (M−1).

2′-Chlorobiphenyl-4-carboxylic acid[1(S)-[4-cyano-1,1-dioxohexahydro-1λ⁶-thiopyran-4-ylcarbamoyl]-2-(2,6-difluorophenyl)ethyl]amide (table 2, compound 5).

¹H-NMR (DMSO-d₆): δ 8.853 (1H, d, J=7.6 Hz, NH), 8.629 (1H, s, NH), 7.87(2H, d, J=8.4 Hz), 7.6-7.5 (1H, m), 7.50 (2H, d, J=8.4 Hz), 7.45-7.4(3H, m), 7.3 (1H, d,d,d), 7.02 (2H, d,d,d), 4.77 (1H, d,d,d, J=7.6 Hz), 3.5-3.0 (6H, m), 2.5-2.4 (4H, m). MS: 570.1(M−1), 572.2(M+1), 594.3(M+Na).

### Example 10

### Synthesis of 2′-chlorobiphenyl-4-carboxylic acid[1(S)-(cyanomethylcarbamoyl)-2-thiazol-2-yl-ethyl]-amide (table 1, compound 32)

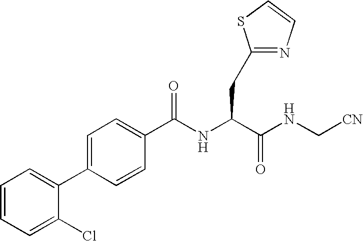 Step 1

Zinc foil (3.27 g, 50 mmol) was activated in anhydrous DMF (10 mL) using the literature procedure (see Knochel, P. et al. J. Org. Chem, 1988, 239) and a solution of (S)-2-tert-butoxycarbonylamino-3-iodo-propionic acid methyl ester (3.29 g, 10 mmol) (see Jackson, R. F. W. et al. J. Org. Chem. 1992, 57, 3397) in DMF (5 mL) was added at room temperature dropwise via a cannula over 10 min., and the reaction mixture was stirred in 1 h. The resulting organozinc solution was then transferred to a 25 mL of round-bottom flask via a cannula. 2-Bromothiazole (1.97 g, 12 mmol) and Pd(PPh₃)₂Cl₂ (0.70, 1.0 mmol) were added successively, and the reaction mixture was stirred at room temperature for 1 h and then at 50° C. for 16 h. The reaction mixture was quenched with saturated aqueous NH₄Cl solution (25 mL). Extraction with ethyl acetate, drying, and concentration gave the crude product, which was purified by flash chromatography on silica gel (eluted with 1:2 EtOAc/hexanes) to afford (S)-2-tert-butoxycarbonylamino-3-thiazol-2-yl-propionic acid methyl ester (1.35 g).

Step 2

Following the procedure described in Example 2, Step 2, 2(S)-tert-butoxycarbonylamino-3-thiazol-2-yl-propionic acid was prepared from 2(S)-tert-butoxycarbonylamino-3-thiazol-2-yl-propionic acid methyl ester in 99% yield.

Step 3

Following the procedure described in Example 2, Step 3, [1(S)-(cyanomethylcarbamoyl)-2-thiazol-2-yl-ethyl]-carbamic acid tert-butyl ester was prepared from 2(S)-tert-butoxycarbonylamino-3-thiazol-2-yl-propionic acid in 86% yield.

Step 4

Following the procedure described in Example 2, Step 4, 2(S)-amino-N-cyanomethyl-3-thiazol-2-yl-propionamide was prepared from[1(S)-(cyanomethylcarbamoyl)-2-thiazol-2-ylethyl]-carbamic acid tert-butyl ester in 90% yield.

Step 5

Following the procedure described in Example 2, Step 5, the title compound was prepared from 2(S)-amino-N-cyanomethyl-3-thiazol-2-yl-propionamide and 2′-chloro-biphenyl-4-carboxylic acid in 66% yield. ¹H NMR (400 MHz, DMSO-d₆): δ 8.97 (1H, J=8.4 Hz), 8.8 (1H, t, J=5.2 Hz), 7.93 (2H, d, J=8.4 Hz), 7.69 (1H, d, J=3.2 Hz), 7.57 (m, 1H), 7.54 (1H, d, J=3.2 Hz), 7.52 (2H, d, J=8.4 Hz), 7.43 (3H, m), 4.88 (1H, ddd, J=10.0, 8.0, 4.0 Hz), 4.14 (2H, m), 3.58 (1H, dd, J=14.8, 4.0 Hz), 3.47 (1H, dd, J=14.8, 10.0 Hz). MS: 425.0(MH⁺).

Following the procedure described in Example 2, Step 5 above, 6-(2-chlorophenyl)-N-[1(S)-(cyanomethylcarbamoyl)-2-thiazol-2-yl-ethyl]-nicotinamide was prepared from 2(S)-amino-N-cyanomethyl-3-thiazol-2-yl-propionamide and 6-(2-chlorophenyl)nicotinic acid in 70% yield (table 1, compound 33).

¹H NMR (400 MHz, DMSO-d₆): δ 9.19 (1H, J=8.4 Hz), 9.06 (1H, m), 8.85 (1H, t, J=5.6 hz), 8.26 (1H, dd, J=8.4, 2.4 Hz), 7.77(1H, d, J=8.4 Hz), 7.69 (1H, d, J=3.2 Hz), 7.58 (m, 1H), 7.55 (1H, d, J=3.2 Hz), 7.47 (2H, d, J=8.4 Hz), 7.43 (3H, m), 4.91 (1H, ddd, J=10.0, 8.4, 4.0 Hz), 4.16 (2H, m), 3.60 (1H, dd, J=14.8, 4.0 Hz), 3.47 (1H, dd, J=14.8, 10.0 Hz). MS: 426.0(MH⁺).

### Example 11

### Synthesis of 2′-chlorobiphenyl-4-carboxylic acid[1(S)-(cyanomethylcarbamoyl)-3(R)-phenyl-butyl]amide (table 1, compound 13)

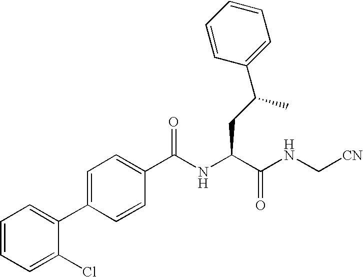 Step 1

To a −30° C. solution of triethylamine (12.3 mL, 88.0 mmol) in dichloromethane (175 mL) was added methanesulfonyl chloride (3.75 mL, 48.4 mmol), then 2(S)-phenylpropanol (6 g, 44 mmol). The reaction mixture was allowed to stir for 1 h at −30° C., then warmed to −10° C. for 5 h. The reaction mixture was poured into 1N HCl (200 mL) and then extracted twice with dichloromethane. The organic phase was washed with 1N HCl and water, dried over magnesium sulfate and concentrated. The crude product was dissolved in acetone (225 mL), treated with sodium iodide (7.86 g, 52.8 mmol) and heated at reflux for 16 h. The reaction mixture was filtered, concentrated and partitioned between diethyl ether and water. The organic phase was washed with sodium bisulfite and brine, and was dried over magnesium sulfate and concentrated. Purification by fluorosil chromatography (hexanes) gave (2-iodo-1-methylethyl)benzene (4.38 g). (2-Iodo-1-methylethyl)benzene was converted to 2-[(2′-chloro-biphen-4-carbonyl)amino]-4-phenylpentanoic acid by following the procedures described in Example I, Steps 2 and 3, followed by procedures described in Example K, Step 4, followed by procedures described in Example M, Steps 2, 3, 4, and 6, followed by procedures described in Example K, Steps 4 and 5. 2-[(2′-Chloro-biphen-4-carbonyl)amino]-4-phenylpentanoic acid was converted to the title compound as described in Example 1 above.

H¹ NMR(CDCl₃): 7.88 (1H, t, J=5.4 Hz), 7.68 (2H, d, J=8.2 Hz), 7.45(3H, m), 7.20 (9H, m), 4.49 (1H, m), 3.96 (2H, m), 2.97 (1H, q, J=7.1 Hz), 2.16 (2H, m), 1.28 (3H, d, J=6.9 Hz)

MS: 444.4 (M−1), 446.2 (M+1).

### Biological Examples

### Example 1

### Cathepsin B Assay

Solutions of test compounds in varying concentrations were prepared in 10 μL of dimethyl sulfoxide (DMSO) and then diluted into assay buffer (40 μL, comprising:NN-bis(2-hydroxyethyl)-2-aminoethanesulfonic acid (BES), 50 mM (pH 6); polyoxyethylenesorbitan monolaurate, 0.05%; and dithiothreitol (DTT), 2.5 mM). Human cathepsin B (0.025 pMoles in 25 μL of assay buffer) was added to the dilutions. The assay solutions were mixed for 5-10 seconds on a shaker plate, covered and incubated for 30 minutes at room temperature. Z-FR-AMC (20 nMoles in 25 μL of assay buffer) was added to the assay solutions and hydrolysis was followed spectrophotometrically at (λ 460 nm) for 5 minutes. Apparent inhibition constants (Kᵢ) were calculated from the enzyme progress curves using standard mathematical models.

Compounds of the invention were tested by the above-described assay and observed to exhibit cathepsin B inhibitory activity.

### Example 2

### Cathepsin K Assay

Solutions of test compounds in varying concentrations were prepared in 10 μL of dimethyl sulfoxide (DMSO) and then diluted into assay buffer (40 μL, comprising: MES, 50 mM (pH 5.5); EDTA, 2.5 mM; and DTT, 2.5 mM). Human cathepsin K (0.0906 pMoles in 25 μL of assay buffer) was added to the dilutions. The assay solutions were mixed for 5-10 seconds on a shaker plate, covered and incubated for 30 minutes at room temperature. Z-Phe-Arg-AMC (4 nMoles in 25 μL of assay buffer) was added to the assay solutions and hydrolysis was followed spectrophotometrically at (λ 460 nm) for 5 minutes. Apparent inhibition constants (Kᵢ) were calculated from the enzyme progress curves using standard mathematical models.

Compounds of the invention were tested by the above-described assay and observed to exhibit cathepsin K inhibitory activity.

### Example 3

### Cathepsin L Assay

Solutions of test compounds in varying concentrations were prepared in 10 μL of dimethyl sulfoxide (DMSO) and then diluted into assay buffer (40 μL, comprising: MES, 50 mM (pH 5.5); EDTA, 2.5 mM; and DTT, 2.5 mM). Human cathepsin L (0.05 pMoles in 25 μL of assay buffer) was added to the dilutions. The assay solutions were mixed for 5-10 seconds on a shaker plate, covered and incubated for 30 minutes at room temperature. Z-Phe-Arg-AMC (1 nMoles in 25 μL of assay buffer) was added to the assay solutions and hydrolysis was followed spectrophotometrically at (λ460 nm) for 5 minutes. Apparent inhibition constants (Kᵢ) were calculated from the enzyme progress curves using standard mathematical models.

Compounds of the invention were tested by the above-described assay and observed to exhibit cathepsin L inhibitory activity.

### Example 4

### Cathepsin S Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM); β-mercaptoethanol, 2.5 mM; and BSA, 0.00%. Human cathepsin S (0.05 pMoles in 25 μL of assay buffer) was added to the dilutions. The assay solutions were mixed for 5-10 seconds on a shaker plate, covered and incubated for 30 minutes at room temperature. Z-Val-Val-Arg-AMC (4 nMoles in 25 μL of assay buffer containing 10% DMSO) was added to the assay solutions and hydrolysis was followed spectrophotometrically (at λ 460 nm) for 5 minutes. Apparent inhibition constants (Kᵢ) were calculated from the enzyme progress curves using standard mathematical models.

Compounds of the invention were tested by the above-described assay and observed to exhibit cathepsin S inhibitory activity.

### Example 5

### Cathepsin F Assay

Solutions of test compounds in varying concentrations were prepared in 10 μL of dimethyl sulfoxide (DMSO) and then diluted into assay buffer (40 μL, comprising: MES, 50 mM (pH 6.5); EDTA, 2.5 mM; and NaCl, 100 mM); DTT, 2.5 mM; and BSA, 0.01%. Human cathepsin F (0.1 pMoles in 25 μL of assay buffer) was added to the dilutions. The assay solutions were mixed for 5-10 seconds on a shaker plate, covered and incubated for 30 minutes at room temperature. Z-Phe-Arg-AMC (2 nMoles in 25 μL of assay buffer containing 10% DMSO) was added to the assay solutions and hydrolysis was followed spectrophotometrically (at λ 460 nm) for 5 minutes. Apparent inhibition constants (Kᵢ) were calculated from the enzyme progress curves using standard mathematical models.

Compounds of the invention were tested by the above-described assay and observed to exhibit cathepsin F inhibitory activity.

### Pharmaceutical Composition Examples

The following are representative pharmaceutical formulations containing a compound of Formula (I).

### Tablet Formulation

The following ingredients are mixed intimately and pressed into single scored tablets.

| Ingredient | Quantity per tablet, mg |
|---|---|
| compound of this invention | 400 |
| cornstarch | 50 |
| croscarmellose sodium | 25 |
| lactose | 120 |
| magnesium stearate | 5 |

### Capsule Formulation

The following ingredients are mixed intimately and loaded into a hard-shell gelatin capsule.

| Ingredient | Quantity per capsule, mg |
|---|---|
| compound of this invention | 200 |
| lactose, spray-dried | 148 |
| magnesium stearate | 2 |

### Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.5 | g |
| sorbitol (70% solution) | 12.85 | g |
| Veegum K (Vanderbilt Co.) | 1.0 | g |
| flavoring | 0.035 | mL |
| colorings | 0.5 | mg |
| distilled water | q.s. to 100 | mL |

### Injectable Formulation

The following ingredients are mixed to form an injectable formulation.

| Ingredient | Amount |
|---|---|
| compound of this invention | 1.2 g |
| sodium acetate buffer solution, | 0.4 M 2.0 mL |
| HCl (1 N) or NaOH (1 N) | q.s. to suitable pH |
| water (distilled, sterile) | q.s. to 20 mL |

All of the above ingredients, except water, are combined and heated to 60-70.degree. C. with stirring. A sufficient quantity of water at 60° C. is then added with vigorous stirring to emulsify the ingredients, and water then added q.s. to 100 g.

### Suppository Formulation

A suppository of total weight 2.5 g is prepared by mixing the compound of the invention with Witepsol.RTM. H-15 (triglycerides of saturated vegetable fatty acid; Riches-Nelson, Inc., New York), and has the following composition:

| | |
|---|---|
| compound of the invention | 500 mg |
| Witepsol ® H-15 | balance |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled. This application claims priority to and incorporates herein by reference the disclosure of U.S. Provisional Application Ser. No. 60/431,354, filed on Dec. 5, 2002, titled “Cyanomethyl derivatives as cysteine protease inhibitors” by Graupe et al. and all the other references cited herein in their entirety.

What is claimed:

1. A compound of Formula (I): 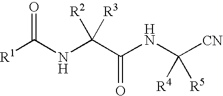
wherein:
R¹ is a group of formula: 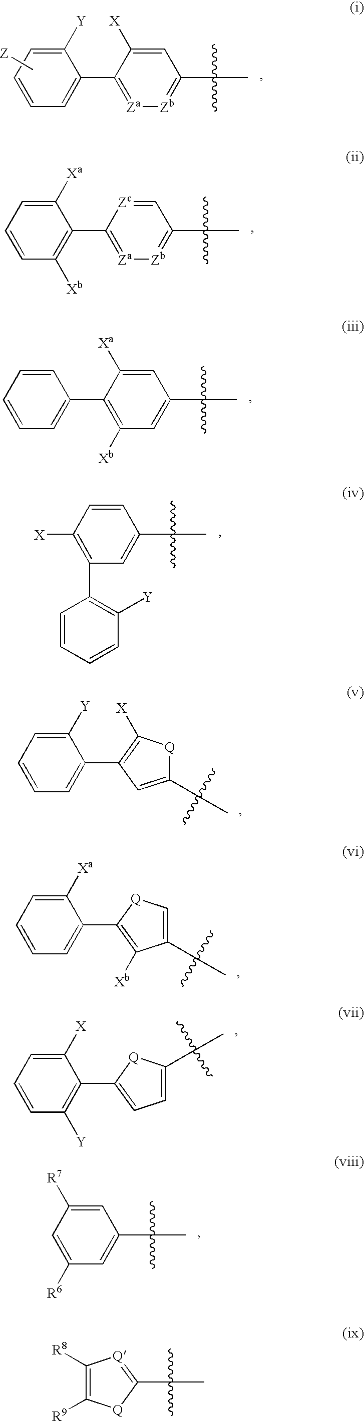 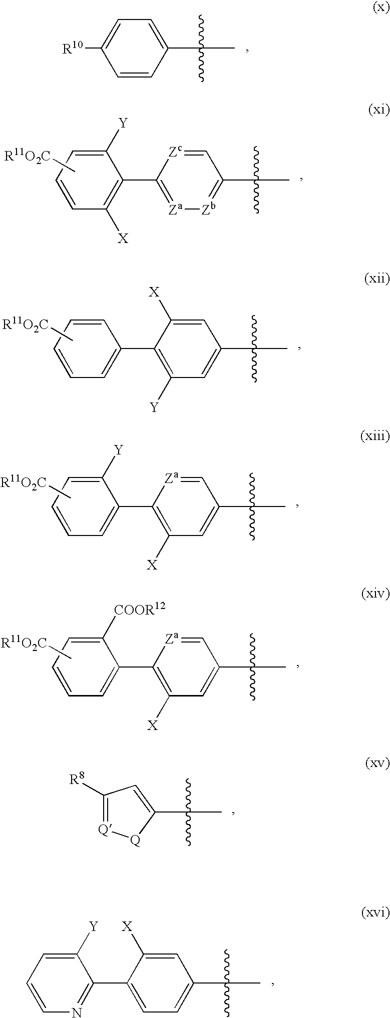
(xvii) 1-(4-aminosulfonylphenyl)-5-(4-chlorophenyl)pyrazol-3-yl;
(xviii) 1-methyl-1H-thieno[2,3-c]pyrazol-5-yl where the 3-position of the pyrazole ring is substituted with alkyl, haloalkyl, or phenyl optionally substituted with alkyl, halo, haloalkyl, haloalkoxy, or alkoxy;
(xix) 4-(3,5-dimethyloxazol-4-yl)phenyl; or
(xx) 4-(5-carboxy-2-methylthiophen-3-yl)phenyl;
(xxi) biphen-4-yl;
(xxii) 4-alkoxycarbonylbiphen-4-yl;
(xxii) 4-carboxybiphen-4-yl;
(xxiii) 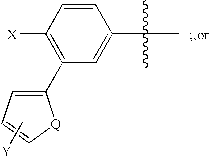
(xxiv) 4-(5-carboxy-2-halothiophen-3-yl)phenyl;
where:
Z^{a} and Z^{b} are independently —CX— or —N— and Z^{c} is selected from —CH— and —N— provided that if an R¹ group contains Z^{a}, Z^{b}, and Z^{c} simultaneously then, when Z^{c} is —N—, then Z^{a} is —N— or —CX— and Z^{b} is —H—; and when Z^{b} is —N— then both Z^{a} and Z^{c} cannot be —N— simultaneously;
Q is —NR— where R is hydrogen or alkyl, —O—, or —S—;
Q′ is —CH— or —N—;
X and Y are independently selected from hydrogen, halo, alkyl, alkoxy, haloalkyl, or haloalkoxy provided that both X and Y are not simultaneously hydrogen;
Z is hydrogen, halo, alkyl, alkoxy, haloalkyl, or haloalkoxy;
X^{a} and X^{b} are independently selected from alkyl, halo, alkoxy, haloalkyl, or haloalkoxy;
R² is selected from the group consisting of hydrogen, cyclopentyl, cyclohexyl, cycloheptyl, methyl, ethyl, n-propyl, 2-propyl, 2-methylpropyl, 2-ethylbutyl, 3-methylbutyl, thiazolylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2,4,4-trimethylpentyl, 1-methylindol-3-ylmethyl, 4-methylindol-3-ylmethyl, 2-napth-1-ylpropyl, benzyloxymethyl, 2-cyclohexylpropyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), benzyl (where the phenyl ring in the benzyl group is optionally substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino; or dialkylamino), heteroaryl(C₃₋₆)alkyl, and 1-heteroaryl(C₃₋₆)cycloalkylmethyl, and furthermore wherein the alkyl chain in the above groups is optionally substituted with one to six halo;
R³ is hydrogen; or
R² and R³ together with the carbon atom to which they are attached form (C₄₋₈)-cycloalkylene, (C₄₋₈)cycloalkenylene or spirocycloalkylene wherein said (C₄₋₈)cycloalkylene, (C₄₋₈)cycloalkenylene or spirocycloalkylene is optionally substituted with one or two alkyl, alkylidene, or alkenyl;
R⁴ is hydrogen;
R⁵ is hydrogen, alkyl or heteroaryl optionally substituted with alkyl, halo, haloalkyl, haloalkoxy, or alkoxy; or
R⁴ and R⁵ together with the carbon atom to which they are attached form cycloalkylene or heterocycloalkylene;
R⁶ and R⁷ are independently selected from phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl;
R⁸ is phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl;
R⁹ is halo, phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl;
R¹⁰ is a branched alkyl chain of 4-6 carbon atoms or trifluoroalkoxy; and
each R¹¹ and R¹² are independently hydrogen or alkyl;
or a pharmaceutically acceptable salt thereof.

2. The compound of claim 1 wherein:
wherein:
R¹ is a group of formula: 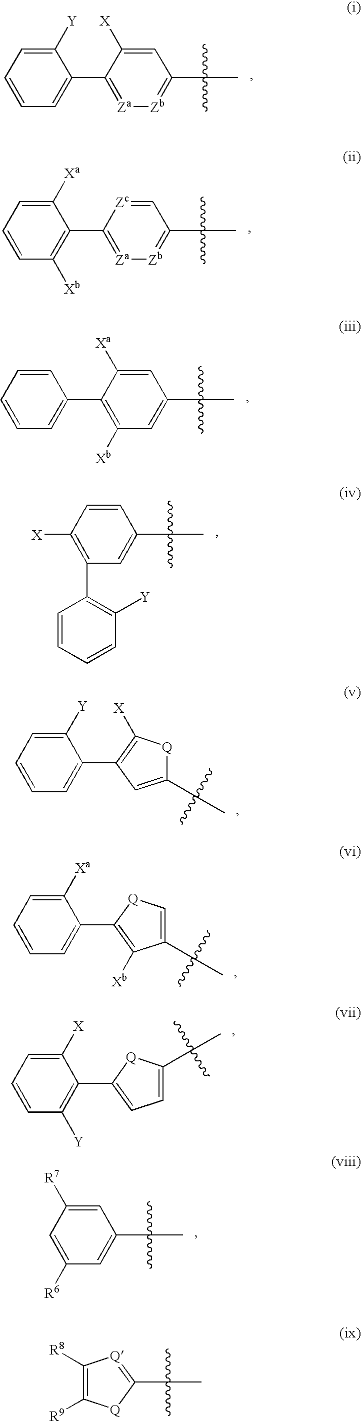 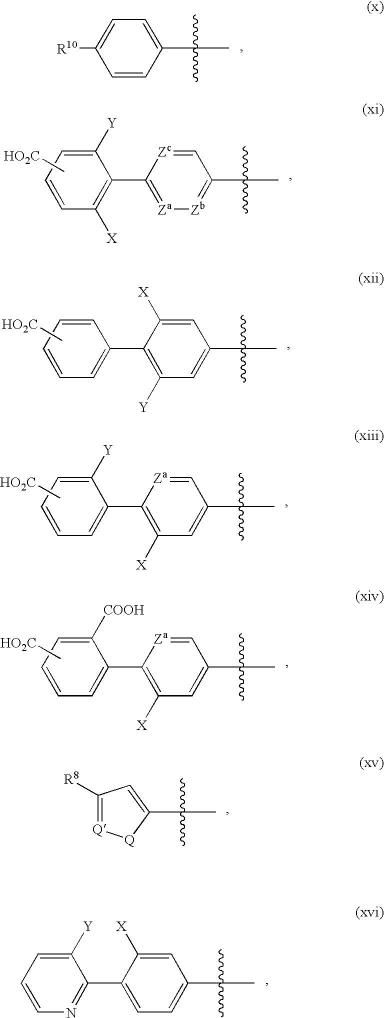
(xvii) 1-(4-aminosulfonylphenyl)-5-(4-chlorophenyl)pyrazol-3-yl;
(xviii) 1-methyl-3-trifluoromethyl-1H-thieno[2,3-c]pyrazol-5-yl;
(xix) 4-(3,5-dimethyloxazol-4-yl)phenyl; or
(xx) 4-(5-carboxy-2-methylthiophen-3-yl)phenyl;
where:
Z^{a} is —CX— or —N— and Z^{b} and Z^{c} are independently selected from —CH— and —N— provided that if an R¹ group contains Z^{a}, Z^{b}, and Z^{c} simultaneously then, when Z^{c} is —N—, then Z^{a} is —N— or —CX— and Z^{b} is —CH—; and when Z^{b} is —N— then both Z^{a} and Z^{c} cannot be —N— simultaneously;
Q is —NR— where R is hydrogen or alkyl, —O—, or —S—;
Q′ is —CH— or —N—;
X and Y are independently selected from hydrogen, halo, alkyl, alkoxy, haloalkyl, or haloalkoxy provided that both X and Y are not simultaneously hydrogen;
X^{a} and X^{b} are independently selected from alkyl, halo, alkoxy, haloalkyl, or haloalkoxy;
R² is selected from the group consisting of hydrogen, cyclopentyl, cyclohexyl, cycloheptyl, methyl, ethyl, n-propyl, 2-propyl, 2-methylpropyl, 2-ethylbutyl, 3-methylbutyl, thiazolylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), benzyl (where the phenyl ring in the benzyl group is optionally substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino), heteroaryl(C₃₋₆)alkyl, and 1-heteroaryl(C₃₋₆)cycloalkylmethyl, and furthermore wherein the alkyl chain in the above groups is optionally substituted with one to six halo;
R³ is hydrogen; or
R² and R³ together with the carbon atom to which they are attached form (C₄₋₈)cycloalkylene, (C₄₋₈)cycloalkenylene or spirocycloalkylene wherein said (C₄₋₈)cycloalkylene, (C₄₋₈)cycloalkenylene or spirocycloalkylene is optionally substituted with one or two alkyl, alkylidene, or alkenyl;
R⁴ is hydrogen;
R⁵ is hydrogen or alkyl; or
R⁴ and R⁵ together with the carbon atom to which they are attached form cycloalkylene or heterocycloalkylene;
R⁶ and R⁷ are independently selected from phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl;
R⁸ and R⁹ are independently selected from phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl; and
R¹⁰ is a branched alkyl chain of 4-6 carbon atoms or trifluoroalkoxy;
or a pharmaceutically acceptable salt thereof.

3. The compound of claim 1 or 2 wherein R³, R⁴ and R⁵ are hydrogen and R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino.

4. The compound of claim 1 or 2 wherein R³ is hydrogen and R⁴ and R⁵ together with the carbon atom to which they are attached form cycloalkylene and R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino.

5. The compound of claim 1 or 2 wherein R³ is hydrogen and R⁴ and R⁵ together with the carbon atom to which they are attached form cyclopropylene and R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino.

6. The compound of claim 1 or 2 wherein R³ is hydrogen and R⁴ and R⁵ together with the carbon atom to which they are attached form heterocycloalkylene and R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino.

7. The compound of claim 1 or 2 wherein R³ is hydrogen, R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino, and R⁴ and R⁵ together with the carbon atom to which they are attached form: 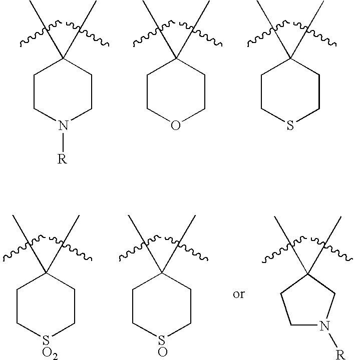
wherein R is hydrogen, alkyl, haloalkyl or cycloalkyl.

8. The compound of claim 7 wherein R⁴ and R⁵ together with the carbon atom to which they are attached form: 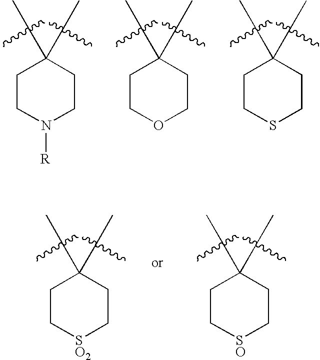
wherein R is methyl, ethyl, 2,2,2-trifluoroethyl, or cyclopropyl.

9. The compound of any of the claims 1-8 wherein R² is 2,6-difluorobenzyl, 2,6-difluoro-4-methoxybenzyl, 2(S)-phenylpropyl, 2(R)-phenylpropyl, 2-methylpropyl, 2-methyl-2-phenylpropyl, 2-phenylethyl, 2-phenylprop-2-enyl, benzyl, or thiazol-2-ylmethyl.

10. The compound of any of the claims 1-8 wherein R¹ is: 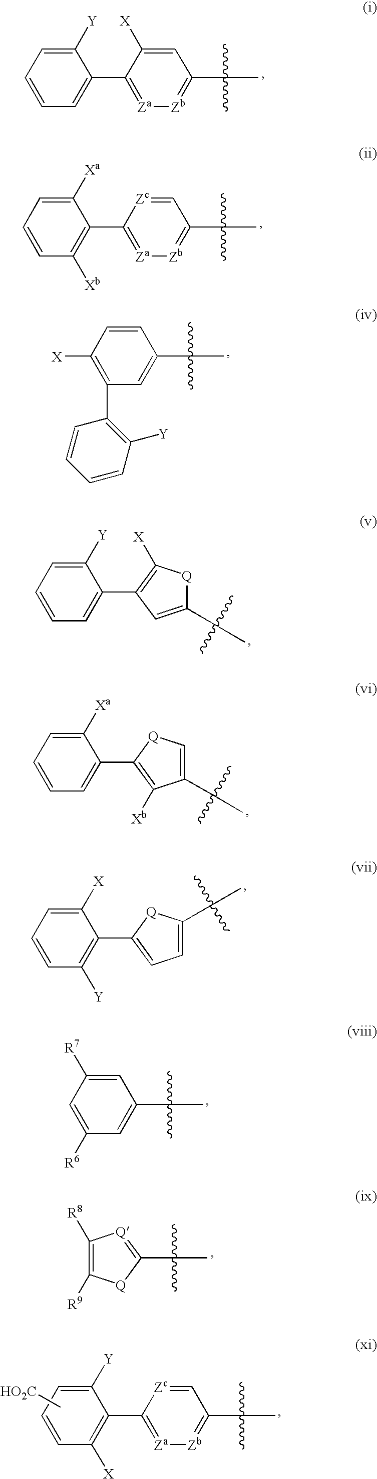 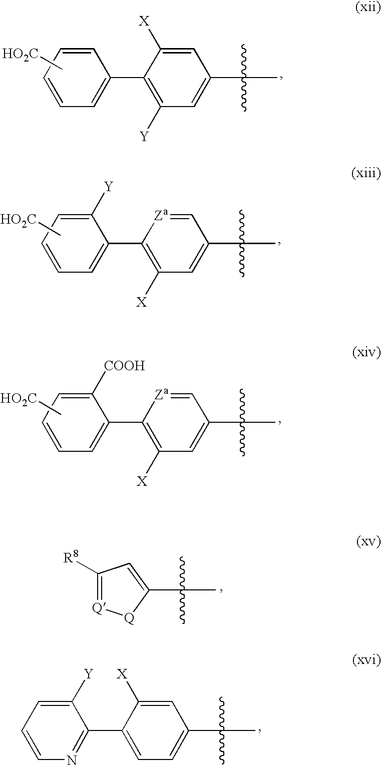
(xvii) 1-(4-aminosulfonylphenyl)-5-(4-chlorophenyl)pyrazol-3-yl;
(xix) 4-(3,5-dimethyloxazol-4-yl)phenyl; or
(xx) 4-(5-carboxy-2-methylthiophen-3-yl)phenyl;
wherein:
X is hydrogen, chloro, methyl, methoxy, trifluoromethyl, or trifluoromethoxy;
Y is chloro, methyl, methoxy, trifluoromethyl, or trifluoromethoxy;
X^{a}, and X^{b} are independently selected from methyl, chloro, fluoro, methoxy, trifluoromethyl, or trifluoromethoxy;
R⁶ is phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl;
R⁷ is 2-alkoxyphenyl, 3-alkoxyphenyl, 2-halophenyl, 2-alkylphenyl, 2-haloalkylphenyl, or 2-haloalkoxyphenyl;
R⁸ is 2-alkoxyphenyl, 3-alkoxyphenyl, 2-halophenyl, 2-alkylphenyl, 2-haloalkylphenyl, or 2-haloalkoxyphenyl; and
R⁹ is phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl.

11. The compound of claim 10 wherein R² is preferably selected from the group consisting of cyclohexyl, cycloheptyl, thiazol-2-ylmethyl, 2-ethylbutyl, pyrazol-1-ylmethyl, 2-methylpropyl, 2,4,4-trimethylpentyl, 2-napth-1-ylpropyl, 2-phenylprop-2-enyl, 2-phenyl-2-methylpropyl, 2-phenylpropyl, 2-methyl-2-(2-methoxyphenyl)propyl, 2-(2-methoxyphenyl)propyl, 4-methylindol-3-ylmethyl, 2-(2,5-dimethylphenyl)propyl, benzyloxymethyl, 2-(2,4-dimethylphenyl)propyl, 2-(2,4-dichlorophenyl)-propyl, 2,6-difluorobenzyl, 2,5-difluorobenzyl, 2,6-difluoro-4-methoxybenzyl, and 2,3-difluorobenzyl.

12. The compound of claim 10 wherein R² is 2,6-difluorobenzyl, 2,6-difluoro-4-methoxybenzyl, 2(S)-phenylpropyl, 2(R)-phenylpropyl, 2-methylpropyl, 2-methyl-2-phenylpropyl, 2-phenylethyl, 2-phenylprop-2-enyl, benzyl, or thiazol-2-ylmethyl.

13. The compound of claim 10, 11 or 12 wherein R¹ is 2′-chlorobiphen-4-yl, 3,2′-dichlorobiphen-4-yl, 2′,6′-dichlorobiphen-4-yl, 2′,6′-dimethylbiphen-4-yl, 2′-methylbiphen-4-yl, 2′-fluorobiphen-4-yl; 2-(2-methylphenyl)furan-5-yl, 2-(2-methoxyphenyl)furan-5-yl, 3-methoxy-2-(2-methylphenyl)thiophen-4-yl, 3-methoxy-2-(2-methoxyphenyl)thiophen-4-yl, 2,3-di(2-methoxyphenyl)thiophen-5-yl, 3,5-di(2-methoxyphenyl)phenyl, 3,5-di(3-methoxyphenyl)phenyl, 2,3-di(2-methylphenyl)thiophen-5-yl, 4-(2-methylphenyl)thiophen-2-yl, 4-(2-methoxyphenyl)thiophen-2-yl, 2′-chlorobiphen-3-yl, 2′-methyl-4-chlorobiphen-3-yl, 3,5-di(2-chlorophenyl)phenyl, 2,3-di(2-chlorophenyl)thiophen-5-yl, 1-(4-aminosulfonylphenyl)-5-(4-chlorophenyl)pyrazol-3-yl, 2-(2,6-dichlorophenyl)furan-5-yl, 3-trifluoromethyl-1-methylthieno[2,3-c]pyrazol-5-yl, 2′-methoxybiphen-4-yl, 2′-trifluoromethylbiphen-4-yl, 2′-methyl-3-chlorobiphen-4-yl, 2-(2-chlorophenyl)pyridin-5-yl, 2-(2,6-dichlorophenyl)pyridin-5-yl, 2-(2-trifluoromethylphenyl)pyridin-5-yl, 4-(3-methylpyridin-2-yl)phenyl, 2-(2-chlorophenyl)-3-chloropyridin-5-yl, 2-(2,6-dichlorophenyl)-3-chloropyridin-5-yl, 4′-carboxy-2′-chlorobiphen-4-yl, 4′-carboxy-2′-fluorobiphen-4-yl, 4′-carboxy-2′-methylbiphen-4-yl, 5′-carboxy-2′-chlorobiphen-4-yl, 5′-carboxy-2′-methylbiphen-4-yl, 2-(4-carboxy-2-chlorophenyl)pyridin-5-yl, 2-(5-carboxy-2-chlorophenyl)pyridin-5-yl, 4-(5-carboxy-2-methylthiophen-3-yl)phenyl, 4-(3-methoxy-phenyl)thiophen-2-yl, 3-(2-chlorophenyl)isoxazol-5-yl, or 4-(3-methylpyridin-2-yl)phenyl, 4-(2-chlorophenyl)thiophen-2-yl, 3-chloro-2′-methylbiphen-4-yl, 1-oxo-2-(2,6-dichlorophenyl)pyridin-5-yl, 1-oxo-2-(2-methylphenyl)pyridin-5-yl, 4′-carboxy-2′-methylbiphen-4-yl, 1-oxo-3-chloro-2-(2-chlorophenyl)pyridin-5-yl, 3-chloro-2-(2-trifluoromethylphenyl)pyridin-S-yl, 3-chloro-2-(2-methylphenyl)pyridin-5-yl, 1-oxo-2-(2-chlorophenyl)pyridin-5-yl, 4′-carboxy-2′6′-dichlorobiphen-4-yl, or 4′-carboxy-3,2′-dichlorobiphen-4-yl.

14. The compound of any of the claims 1-8 wherein R¹ is a group of formula: 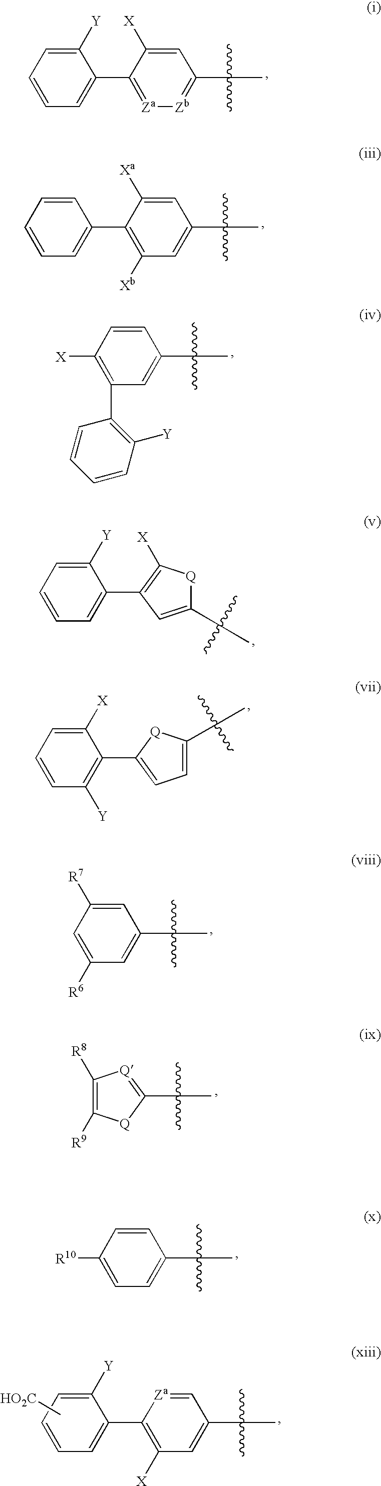 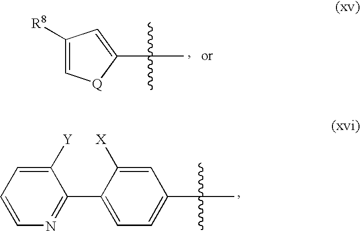
where:
X is chloro, methyl, methoxy, trifluoromethyl, or trifluoromethoxy;
Y is hydrogen;
X^{a}, and X^{b} are independently selected from methyl, chloro, fluoro, methoxy, trifluoromethyl, or trifluoromethoxy;
R⁶ and R⁷ are phenyl;
R⁸ are independently selected from phenyl, furan-2-yl, thiophen-3-yl, or pyridin-4-yl;
R⁹ is phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl; and
R¹⁰ is a branched alkyl chain of 4-6 carbon atoms or trifluoroalkoxy.

15. The compound of claim 14 wherein R² is preferably selected from the group consisting of cyclohexyl, cycloheptyl, thiazol-2-ylmethyl, 2-ethylbutyl, pyrazol-1-ylmethyl, 2-methylpropyl, 2,4,4-trimethylpentyl, 2-napth-1-ylpropyl, 2-phenylprop-2-enyl, 2-phenyl-2-methylpropyl, 2-phenylpropyl, 2-methyl-2-(2-methoxyphenyl)propyl, 2-(2-methoxyphenyl)propyl, 4-methylindol-3-ylmethyl, 2-(2,5-dimethylphenyl)propyl, benzyloxymethyl, 2-(2,4-dimethylphenyl)propyl, 2-(2,4-dichlorophenyl)-propyl, 2,6-difluorobenzyl, 2,5-difluorobenzyl, 2,6-difluoro-4-methoxybenzyl, and 2,3-difluorobenzyl.

16. The compound of claim 14 wherein R² is 2,6-difluorobenzyl, 2,6-difluoro-4-methoxybenzyl, 2(S)-phenylpropyl, 2(R)-phenylpropyl, 2-methylpropyl, 2-methyl-2-phenylpropyl, 2-phenylethyl, 2-phenylprop-2-enyl, benzyl, or thiazol-2-ylmethyl.

17. The compound of claim 14, 15 or 16 wherein R¹ is 4-trifluoromethoxyphenyl, 4-(2-butyl)phenyl, 3,5-diphenylphenyl, 2,3-diphenylthiophen-5-yl, 3,5-di(thiophen-3-yl)phenyl, 3,5-di(pyridin-4-yl)phenyl, 4-tert-butylphenyl, 2,3-di(furan-2-yl)thiophen-5-yl, 3,5-di(furan-2-yl)phenyl, 2,3-diphenylthiophen-5-yl, 4,5-diphenylthiazol-2-yl, or 3-methylbiphen-4-yl.

18. The compound of claim 1 wherein R³, R⁴ and R⁵ are hydrogen and R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino.

19. The compound of claim 1 wherein R³ is hydrogen and R⁴ and R⁵ together with the carbon atom to which they are attached form cycloalkylene and R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino.

20. The compound of claim 1 wherein R³ is hydrogen and R⁴ and R⁵ together with the carbon atom to which they are attached form cyclopropylene and R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino.

21. The compound of claim 1 wherein R³ is hydrogen and R⁴ and R⁵ together with the carbon atom to which they are attached form heterocycloalkylene and R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino.

22. The compound of claim 1 wherein R³ is hydrogen, R² is selected from the group consisting of cyclopentyl, cyclohexyl, cycloheptyl, 2-ethylbutyl, thiazol-2-ylmethyl, pyrazol-1-ylmethyl, 1,2,3-triazol-1-ylmethyl, 1,2,4-triazol-1-ylmethyl, pyrrol-1-ylmethyl, imidazol-1-ylmethyl, tetrazol-1-ylmethyl, 2-pyridin-2-ylpropyl, 2-methyl-2-pyridin-2-ylpropyl, 1-pyridin-2-ylcyclopropylmethyl, 1-pyridin-2-ylcyclobutylmethyl, 2-methylpropyl, 2,4,4-trimethylpentyl, 4-methylindol-3-ylmethyl, 2-napth-1-ylpropyl, benzyloxymethyl, 1-phenylcyclopropylmethyl, 1-phenylcyclobutylmethyl, 2-phenylprop-2-enyl, 2-phenyl-2-methylpropyl, 2-phenylpropyl, 2-phenylbutyl (wherein the phenyl group in 1-phenylcyclopropylmethyl, 1-phenylcyclobutylmethyl, benzyloxymethyl, 2-phenylprop-2-enyl, 2-phenyl-2-methylpropyl, 2-phenylpropyl, or 2-phenylbutyl is optionally substituted with one or two substituents independently selected from alkyl, halo, haloalkoxy, haloalkyl, or alkoxy), and benzyl where the phenyl ring in the benzyl group is substituted at the 2 and 6 positions with groups independently selected from alkyl, halo, haloalkyl, alkoxy or haloalkoxy and at the 4 position with hydrogen, alkyl, halo, haloalkyl, alkoxy, alkoxyalkyloxy, aminoalkyloxy, alkoxyalkylthio, aminoalkylthio, haloalkoxy, alkylthio, alkylsulfinyl, alkylsulfonyl, cyano, amino, alkylamino, or dialkylamino, and R⁴ and R⁵ together with the carbon atom to which they are attached form: 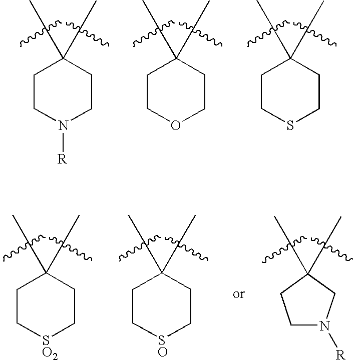
wherein R is hydrogen, alkyl, haloalkyl or cycloalkyl.

23. The compound of claim 1 wherein R⁴ and R⁵ together with the carbon atom to which they are attached form: 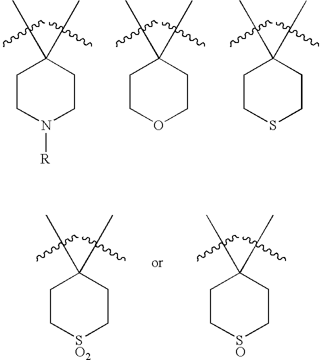
wherein R is methyl, ethyl, 2,2,2-trifluoroethyl, or cyclopropyl.

24. The compound of any of the claims 18-23 wherein R² is 2,6-difluorobenzyl, 2,6-difluoro-4-methoxybenzyl, 2(S)-phenylpropyl, 2(R)-phenylpropyl, 2-methylpropyl, 2-methyl-2-phenylpropyl, 2-phenylethyl, 2-phenylprop-2-enyl, benzyl, or thiazol-2-ylmethyl.

25. The compound of any of the claims 18-23 wherein R¹ is: 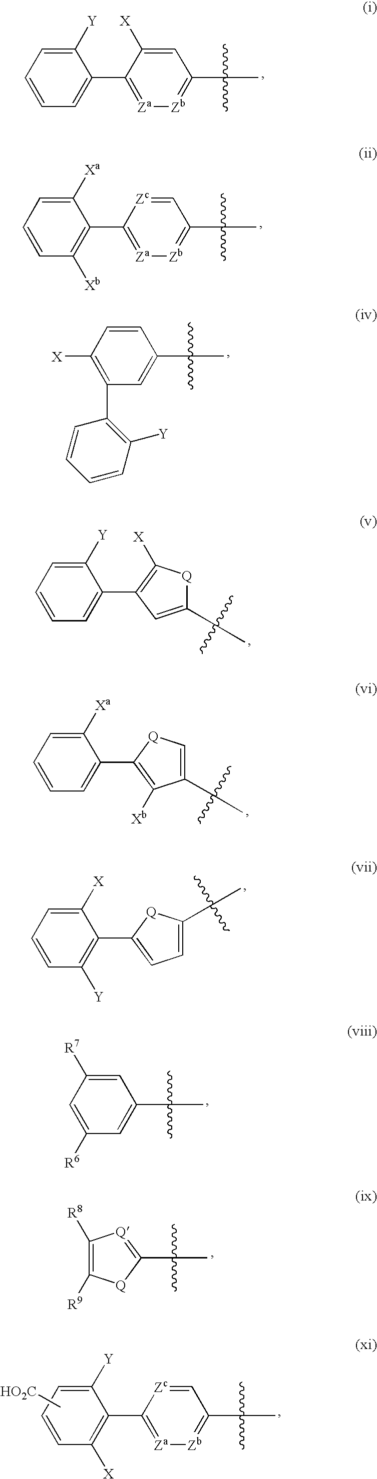 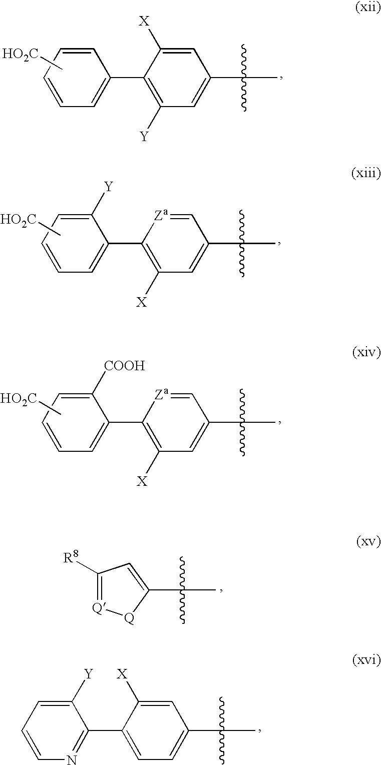
(xvii) 1-(4-aminosulfonylphenyl)-5-(4-chlorophenyl)pyrazol-3-yl;
(xix) 4-(3,5-dimethyloxazol-4-yl)phenyl; or
(xx) 4-(5-carboxy-2-methylthiophen-3-yl)phenyl;
wherein:
X is hydrogen, chloro, methyl, methoxy, trifluoromethyl, or trifluoromethoxy;
Y is chloro, methyl, methoxy, trifluoromethyl, or trifluoromethoxy;
X^{a}, and X^{b} are independently selected from methyl, chloro, fluoro, methoxy, trifluoromethyl, or trifluoromethoxy;
R⁶ is phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl;
R⁷ is 2-alkoxyphenyl, 3-alkoxyphenyl, 2-halophenyl, 2-alkylphenyl, 2-haloalkylphenyl, or 2-haloalkoxyphenyl;
R⁸ is 2-alkoxyphenyl, 3-alkoxyphenyl, 2-halophenyl, 2-alkylphenyl, 2-haloalkylphenyl, or 2-haloalkoxyphenyl; and
R⁹ is phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl.

26. The compound of claim 25 wherein R² is preferably selected from the group consisting of cyclohexyl, cycloheptyl, thiazol-2-ylmethyl, 2-ethylbutyl, pyrazol-1-ylmethyl, 2-methylpropyl, 2,4,4-trimethylpentyl, 2-napth-1-ylpropyl, 2-phenylprop-2-enyl, 2-phenyl-2-methylpropyl, 2-phenylpropyl, 2-methyl-2-(2-methoxyphenyl)propyl, 2-(2-methoxyphenyl)propyl, 4-methylindol-3-ylmethyl, 2-(2,5-dimethylphenyl)propyl, benzyloxymethyl, 2-(2,4-dimethylphenyl)propyl, 2-(2,4-dichlorophenyl)-propyl, 2,6-difluorobenzyl, 2,5-difluorobenzyl, 2,6-difluoro-4-methoxybenzyl, and 2,3-difluorobenzyl.

27. The compound of claim 25 wherein R² is 2,6-difluorobenzyl, 2,6-difluoro-4-methoxybenzyl, 2(S)-phenylpropyl, 2(R)-phenylpropyl, 2-methylpropyl, 2-methyl-2-phenylpropyl, 2-phenylethyl, 2-phenylprop-2-enyl, benzyl, or thiazol-2-ylmethyl.

28. The compound of claim 26 wherein R¹ is 2′-chlorobiphen-4-yl, 3,2′-dichlorobiphen-4-yl, 2′,6′-dichlorobiphen-4-yl, 2′,6′-dimethylbiphen-4-yl, 2′-methylbiphen-4-yl, 2′-fluorobiphen-4-yl; 2-(2-methylphenyl)furan-5-yl, 2-(2-methoxyphenyl)furan-5-yl, 3-methoxy-2-(2-methylphenyl)thiophen-4-yl, 3-methoxy-2-(2-methoxyphenyl)thiophen-4-yl, 2,3-di(2-methoxyphenyl)thiophen-5-yl, 3,5-di(2-methoxyphenyl)phenyl, 3,5-di(3-methoxyphenyl)phenyl, 2,3-di(2-methylphenyl)thiophen-5-yl, 4-(2-methylphenyl)thiophen-2-yl, 4-(2-methoxyphenyl)thiophen-2-yl, 2′-chlorobiphen-3-yl, 2′-methyl-4-chlorobiphen-3-yl, 3,5-di(2-chlorophenyl)phenyl, 2,3-di(2-chlorophenyl)thiophen-5-yl, 1-(4-aminosulfonylphenyl)-5-(4-chlorophenyl)pyrazol-3-yl, 2-(2,6-dichlorophenyl)furan-5-yl, 3-trifluoromethyl-1-methylthieno[2,3-c]pyrazol-5-yl, 2′-methoxybiphen-4-yl, 2′-trifluoromethylbiphen-4-yl, 2′-methyl-3-chlorobiphen-4-yl, 2-(2-chlorophenyl)pyridin-5-yl, 2-(2,6-dichlorophenyl)pyridin-5-yl, 2-(2-trifluoromethylphenyl)pyridin-5-yl, 4-(3-methylpyridin-2-yl)phenyl, 2-(2-chlorophenyl)-3-chloropyridin-5-yl, 2-(2,6-dichlorophenyl)-3-chloropyridin-5-yl, 4′-carboxy-2′-chlorobiphen-4-yl, 4′-carboxy-2′-fluorobiphen-4-yl, 4′-carboxy-2′-methylbiphen-4-yl, 5′-carboxy-2′-chlorobiphen-4-yl, 5′-carboxy-2′-methylbiphen-4-yl, 2-(4-carboxy-2-chlorophenyl)pyridin-5-yl, 2-(5-carboxy-2-chlorophenyl)pyridin-5-yl, 4-(5-carboxy-2-methylthiophen-3-yl)phenyl, 4-(3-methoxy-phenyl)thiophen-2-yl, 3-(2-chlorophenyl)isoxazol-5-yl, or 4-(3-methylpyridin-2-yl)phenyl, 4-(2-chlorophenyl)thiophen-2-yl, 3-chloro-2′-methylbiphen-4-yl, 1-oxo-2-(2,6-dichlorophenyl)pyridin-5-yl, 1-oxo-2-(2-methylphenyl)pyridin-5-yl, 4′-carboxy-2′-methylbiphen-4-yl, 1-oxo-3-chloro-2-(2-chlorophenyl)pyridin-5-yl, 3-chloro-2-(2-trifluoromethylphenyl)pyridin-5-yl, 3-chloro-2-(2-methylphenyl)pyridin-5-yl, 1-oxo-2-(2-chlorophenyl)pyridin-5-yl, 4′-carboxy-2′6′-dichlorobiphen-4-yl, or 4′-carboxy-3,2′-dichlorobiphen-4-yl.

29. The compound of any of the claims 18-23 wherein R¹ is a group of formula: 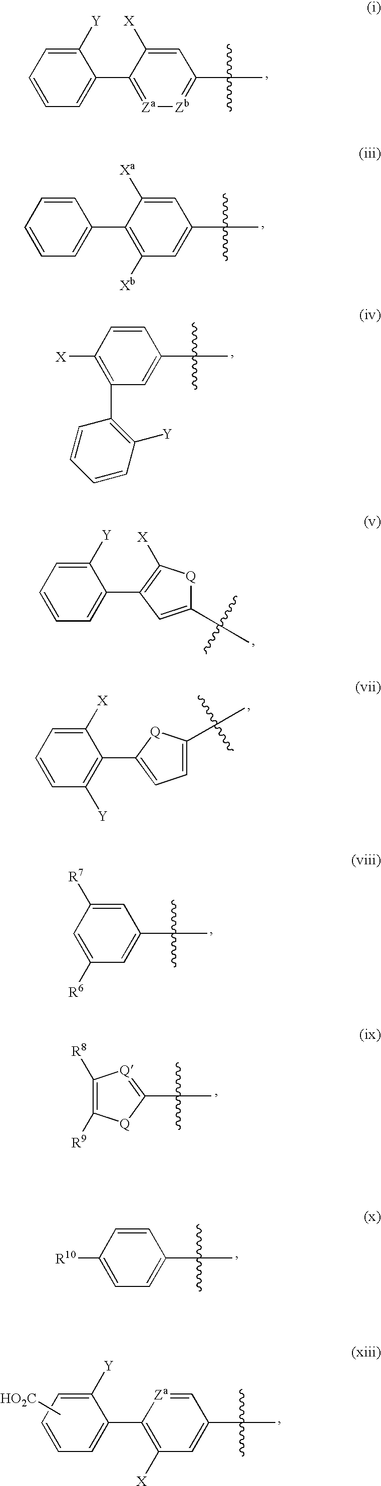 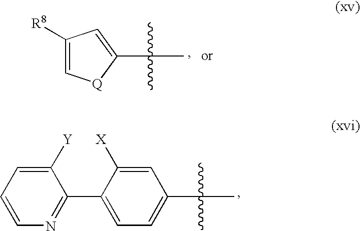
where:
X is chloro, methyl, methoxy, trifluoromethyl, or trifluoromethoxy;
Y is hydrogen;
X^{a}, and X^{b} are independently selected from methyl, chloro, fluoro, methoxy, trifluoromethyl, or trifluoromethoxy;
R⁶ and R⁷ are phenyl;
R⁸ are independently selected from phenyl, furan-2-yl, thiophen-3-yl, or pyridin-4-yl;
R⁹ is phenyl, 2-alkoxyphenyl, 3-alkoxyphenyl, 2-halophenyl, 2-alkylphenyl, 2-haloalkylphenyl, 2-haloalkoxyphenyl, furan-2-yl, thiophen-3-yl, or pyridin-4-yl; and
R¹⁰ is a branched alkyl chain of 4-6 carbon atoms or trifluoroalkoxy.

30. The compound of claim 29 wherein R² is preferably selected from the group consisting of cyclohexyl, cycloheptyl, thiazol-2-ylmethyl, 2-ethylbutyl, pyrazol-1-ylmethyl, 2-methylpropyl, 2,4,4-trimethylpentyl, 2-napth-1-ylpropyl, 2-phenylprop-2-enyl, 2-phenyl-2-methylpropyl, 2-phenylpropyl, 2-methyl-2-(2-methoxyphenyl)propyl, 2-(2-methoxyphenyl)propyl, 4-methylindol-3-ylmethyl, 2-(2,5-dimethylphenyl)propyl, benzyloxymethyl, 2-(2,4-dimethylphenyl)propyl, 2-(2,4-dichlorophenyl)-propyl, 2,6-difluorobenzyl, 2,5-difluorobenzyl, 2,6-difluoro-4-methoxybenzyl, and 2,3-difluorobenzyl.

31. The compound of claim 30 wherein R² is 2,6-difluorobenzyl, 2,6-difluoro-4-methoxybenzyl, 2(S)-phenylpropyl, 2(R)-phenylpropyl, 2-methylpropyl, 2-methyl-2-phenylpropyl, 2-phenylethyl, 2-phenylprop-2-enyl, benzyl, or thiazol-2-ylmethyl.

32. The compound of claim 30 wherein R¹ is 4-trifluoromethoxyphenyl, 4-(2-butyl)phenyl, 3,5-diphenylphenyl, 2,3-diphenylthiophen-5-yl, 3,5-di(thiophen-3-yl)phenyl, 3,5-di(pyridin-4-yl)phenyl, 4-tert-butylphenyl, 2,3-di(furan-2-yl)thiophen-5-yl, 3,5-di(furan-2-yl)phenyl, 2,3-diphenylthiophen-5-yl, 4,5-diphenylthiazol-2-yl, or 3-methylbiphen-4-yl.

33. A pharmaceutical composition comprising a compound of any of the claims 1-33 pharmaceutically acceptable excipient.

34. A method of treating a disease in a patient mediated by cathepsin B, K, L, F, and/or S which method comprises administering to said patient a pharmaceutical composition comprising a compound of any of the claims 1-33 and a pharmaceutically acceptable excipient.

35. The method of claim 34 wherein the disease is Alzheimer's disease, a cardiovascular disease, a respiratory disease, osteroporosis, and an autoimmune disease.

36. The method of claim 34 wherein the disease is asthma, rheumatoid arthritis, systemic lupus erythematosus, Crohn's disease, ulcerative colitis, multiple sclerosis.

37. Use of a compound of any of the claims 1-33 in the preparation of a medicament for the treatment of a disease mediated by Cathepsin B, K, L, F, and/or S.
